SEQUENCE LISTING (16 sequences)

SEQ ID NO: 1 - protein, 8 aa, Artificial Sequence
  Synthesized amino acid
GGGGRGDY

SEQ ID NO: 2 - protein, 14 aa, Artificial Sequence
  Synthesized amino acid
GGGGSPPRRARVTY

SEQ ID NO: 3 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 4 - DNA, 852 bp, Homo sapiens
atgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatga

SEQ ID NO: 5 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 6 - DNA, 1059 bp, Homo sapiens
atggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccaccaccacgccgcccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtag

SEQ ID NO: 7 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 8 - DNA, 1071 bp, Homo sapiens
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatctcgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccgcacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 9 - DNA, 21 bp, Artificial Sequence
  Synthesized DNA
tgcacaggagccaagagtgaa

SEQ ID NO: 10 - DNA, 20 bp, Artificial Sequence
  Synthesized DNA
cacatcacagctccccacca

SEQ ID NO: 11 - DNA, 20 bp, Artificial Sequence
  Synthesized DNA
acccactcctccacctttga

SEQ ID NO: 12 - DNA, 22 bp, Artificial Sequence
  Synthesized DNA
ctgttgctgtagccaaattcgt

SEQ ID NO: 13 - DNA, 21 bp, Artificial Sequence
  Synthesized DNA
aagtctaccaaagctcacgcg

SEQ ID NO: 14 - DNA, 15 bp, Artificial Sequence
  Synthesized DNA
gtaggcgccgcctgc

SEQ ID NO: 15 - DNA, 18 bp, Artificial Sequence
  Synthesized DNA
agcagcggcacattctgg

SEQ ID NO: 16 - DNA, 21 bp, Artificial Sequence
  Synthesized DNA
ttgtacaggtcccgctctttg

(19) United States
(12) Patent Application Publication
(10) Pub. No.: US 2021/0171915 A1
(43) Pub. Date: Jun. 10, 2021

(54) THREE DIMENSIONAL CLUSTERS OF TRANSDIFFERENTIATED CELLS, COMPOSITIONS AND METHODS THEREOF

(71) Applicants: Orgenesis Inc., Germantown, MD (US); B. G. NEGEV TECHNOLOGIES AND APPLICATIONS LTD. AT BEN GURION UNIVERSITY, Beer Sheva (IL); TEL HASHOMER MEDICAL RESEARCH INFRASTRUCTRE AND SERVICES LTD., Ramat Gan (IL)

(72) Inventors: Efrat ASSA KUNIK, Gealya (IL); Smadar COHEN, Beer Sheva (IL); Alon MONSONEGO, Nir-Banim (IL); Sarah FERBER, Tel Aviv (IL)

(73) Assignees: Orgenesis Inc., Germantown, MD (US); B. G. NEGEV TECHNOLOGIES AND APPLICATIONS LTD. AT BEN GURION UNIVERSITY, Beer Sheva (IL); TEL HASHOMER MEDICAL RESEARCH INFRASTRUCTRE AND SERVICES LTD., Ramat Gan (IL)

(21) Appl. No.: 16/978,196

(22) PCT Filed: Mar. 6, 2019
(86) PCT No.: PCT/IL2019/050248

§ 371 Date: Sep. 4, 2020

Related U.S. Application Data
(60) Provisional application No. 62/638,978, filed on Mar. 6, 2018.

(51) Int. Cl. C12N 5/071; A61K 9/00; A61K 35/12; A61K 38/18; A61K 38/20

(52) U.S. Cl. CPC C12N 5/0671; A61K 9/0024; A61K 35/12; A61K 38/1841; C12N 2533/80; A61K 38/2066; C12N 2510/00; C12N 2533/74; A61K 38/1866

(57) ABSTRACT

The present disclosure provides compositions and methods for providing a cell replacement therapy to treat various diseases, including pancreatic diseases and diabetes. Specifically, the disclosure provides three-dimensional (3D) cell clusters of transdifferentiated insulin producing cells attached to scaffolds, such as a polysaccharide matrix, in order to provide a cell replacement therapy.

### FIELD OF THE DISCLOSURE

The disclosure presented herein provides three-dimensional (3D) cell clusters attached to a scaffold, such as a polysaccharide matrix, the clusters comprising transdifferentiated mammalian non-pancreatic beta insulin producing cells (IPCs). Also disclosed herein are methods of generating and methods for treating a pancreatic disorder with said clusters.

### BACKGROUND

Cells in general and pancreatic β-cells in particular exist in three-dimensional (3D) microenvironments with intricate cell-cell and cell-matrix interactions and complex transport dynamics for nutrients and cells. Standard two-dimensional (2D), or monolayer, cell cultures are inadequate representations of this environment. 3D cell clusters more closely resemble in vivo tissue in terms of cellular communication and the development of extracellular matrices. These matrices help cells to function similar to the way cells would function in living tissue. In general, 3D cell cultures also have greater stability and longer lifespans than cell cultures in 2D. This means that they are more suitable for long-term implantation and for long-term effects of the cells on the host. A number of 3D cell culture options are currently available, including scaffold-free platforms, scaffolds, gels, bioreactors, and microchips.

Scaffolds are materials that cause desirable cellular interactions, as the formation of 3D cell clusters. To induce cell adhesion, proliferation, and activation, materials used for the fabrication of scaffolds must possess requirements such as intrinsic biocompatibility and proper chemistry to induce molecular biorecognition from cells. The materials, scaffold mechanical properties and degradation kinetics should be adapted to the specific tissue engineering application to guarantee the required mechanical functions and to accomplish the rate of the new-tissue formation. Specific cell-tissue scaffold formations and topologies provide the ideal environment to support cells functional role and viability.

Polymers represent materials of choice for biological scaffolds. Biomedical polymers are biocompatible, as they comprises the capacity to interact with the organism without causing inflammation or irritation of surrounding tissues. Owing to their origin, natural polymers may positively enhance cell material interactions. However, this origin can potentially induce dangerous immune reactions.

Cell encapsulation represents a further alternative for nesting cell clusters while also providing a protective environment. In cell encapsulation, cells are immobilized within a membrane that permits the bidirectional diffusion of vital molecules such as oxygen, nutrients and growth factors, as well as the outward efflux of waste products and molecules of interest, as therapeutic ones. Cell encapsulation aims to overcome the problem of graft rejection by the host, thus reducing the need of immunosuppressive drugs and improves graft-survival. Additionally, cell encapsulation enables to retrieve the implanted cells in order to follow up their potency in vivo, or in case the cells can cause any risk for the implanted patient.

Diabetes mellitus, commonly referred to as diabetes, is a clinical disorder characterized by the inadequate secretion and/or utilization of insulin resulting in a life-threatening condition that is projected to be the 7th leading cause of death in 2030. Treatment options for diabetes are centered on self-injection of insulin, which is an inconvenient and imprecise solution. Pancreas transplantation is also considered in patients with severe complications of the disease. Although pancreas transplantation is associated with insulin independence in >80% of patients, it is a complicated procedure with significant morbidity and mortality.

Though most of the efforts to develop cell-based therapies for the treatment of diabetes make use of pancreatic islets, an increased research effort has been recently directed at the differentiation of cells from various sources into insulin producing cells (IPC). Reprogramming of adult human liver cells toward IPC by ectopic expression of pancreatic transcription factors (pTF) has been suggested as an unlimited source of β-cell replenishment. Transdifferentiated liver cells were shown to produce, process, and secrete insulin in a glucose-regulated manner, ameliorating hyperglycemia by in vivo implantation in diabetic SCID mice. To achieve insulin secretion, liver cells are transduced with pTF to induce differentiation into glucose regulated insulin-producing cells.

It is clear that there remains a critical need for improved treatment for diabetes. Disclosed herein are 3D cell clusters attached to scaffolds, such as polysaccharide matrices, having several features that make them advantageous over treatments for diabetes known in the art, as well as over other IPC. These clusters may be used in transplantation therapies, obviating the need for numerous self-injections of insulin, now required for the treatment of diabetes.

### SUMMARY OF THE DISCLOSURE

In one aspect, disclosed herein is a three-dimensional (3D) cell cluster comprising transdifferentiated mammalian non-pancreatic beta insulin producing cells (IPC) and a polysaccharide matrix, wherein at least a subset of said cells are attached to said polysaccharide matrix.

In a related aspect, the polysaccharide matrix comprises a sulfated polysaccharide matrix, or a mix of sulfated polysaccharides and polysaccharides. In a related aspect, polysaccharide matrix comprises an alginate polysaccharide. In a related aspect, the sulfated polysaccharide matrix comprises alginate sulfate. In a related aspect, the sulfated polysaccharide matrix comprises hyaluronan sulfate. In a related aspect, the sulfated polysaccharide matrix comprises a bioactive polypeptide associated with a sulfate group of said sulfated polysaccharide matrix. In a related aspect, the sulfated polysaccharide matrix comprises a number of different bioactive polypeptides associated with sulfate groups of said sulfated polysaccharide matrix. In a related aspect, the bioactive polypeptide or the different bioactive polypeptides comprise a positively-charged polypeptide, a heparin-binding polypeptide, or a combination of both.

In a related aspect, the bioactive polypeptide or the number of different bioactive polypeptides are selected from a group comprising antithrombin III (ATIII), thrombopoietin (TPO), serine protease inhibitor (SLP1), CI esterase inhibitor (C1-INH), Vaccinia virus complement control protein (VCP), a fibroblast growth factor (FGF), a FGF receptor, vascular endothelial growth factor (VEGF), hepatocyte growth factor (HGF), insulin-like growth factor (IGF), a platelet-derived growth factor (PDGF), PDGF-ββ, bone morphogenetic protein (BMP), epidermal growth factor (EGF), CXC chemokine ligand 4 (CXCL4), stromal cell-derived factor-1 (SDF-1), interleukin-6 (IL-6), interleukin-8 (IL-8), interleukin-10 (IL-10), Regulated on Activation, Normal T Expressed and Secreted (RANTES), monocyte chemoattractant protein-1 (MCP-1), macrophage inflammatory peptide-1 (MIP-1), lymphotactin, fractalkine, an annexin, apolipoprotein E (ApoE), immunodeficiency virus type-1 (HIV-1) coat protein gp120, cyclophilin A (CypA), Tat protein, viral coat glycoprotein gC, gB or gD of herpes simplex virus (HSV), an envelope protein of Dengue virus, circumsporozoite (CS) protein of Plasmodium falciparum, bacterial surface adhesion protein OpaA, 1-selectin, P-selectin, heparin-binding growth-associated molecule (HB-GAM), thrombospondin type I repeat (TSR), peptide myelin oligodendrocyte glycoprotein (MOG), amyloid P (AP), transforming growth factor (TGF)-β1, or any combination thereof.

In a related aspect, the sulfated polysaccharide matrix comprises four bioactive polypeptides associated with sulfate groups of said sulfated polysaccharide matrix. In a related aspect, the four bioactive polypeptides comprise VEGF, IL-10, PDGF-ββ and TGF-β1. In a related aspect, the association with a sulfate group of the sulfated polysaccharide matrix comprises a non-covalent bond.

In another aspect, the 3D cell cluster is encapsulated by an encapsulation agent. In a related aspect, the encapsulation agent comprises a material selected from a group comprising: alginate, cellulose sulphate, collagen, chitosan, gelatin, agarose, polyethylene glycol (PEG), poly-L-lysine (PLL), polysulphone (PSU), polyvinyl alcohol (PVA), polylactic acid (PLA), acrylates, and low molecular weight dextran sulphate (LMW-DS), or any derivatives thereof, and any combination thereof. In a related aspect, the scaffold encapsulates the transdifferentiated cells.

In another aspect, the transdifferentiated cells comprise improved glucose regulated C-peptide secretion, improved glucose regulated insulin secretion, increased insulin content, increased expression of GCG, increased expression of NKX6.1, or increased expression of PAX6, or any combination thereof, compared to transdifferentiated non-pancreatic beta insulin producing cells cultured as a monolayer cell culture. In another aspect, the transdifferentiated cells comprise improved glucose regulated C-peptide secretion, improved glucose regulated insulin secretion, increased insulin content, increased expression of GCG, increased expression of NKX6.1, or increased expression of PAX6, or any combination thereof, compared to transdifferentiated non-pancreatic beta insulin producing cells cultured as a 3D cell cluster without a scaffold.

In a related aspect, the viability of the transdifferentiated mammalian non-pancreatic beta insulin producing cells is similar to that of transdifferentiated mammalian non-pancreatic beta insulin producing cells cultured as a monolayer cell culture.

In another aspect, the transdifferentiated mammalian non-pancreatic beta insulin producing cells are adult cells. In a related aspect, the transdifferentiated mammalian non-pancreatic beta insulin producing cells are selected from a group comprising: epithelial cells, endothelial cells, keratinocytes, fibroblasts, muscle cells, hepatocytes, liver cells, blood cells, stem or progenitor cells, embryonic heart muscle cells, liver stem cells, neural stem cells, mesenchymal stem cells, hematopoietic stem and progenitor cells, pancreatic cells other than pancreatic beta cells, acinar cells, and alpha-cells, or any combination thereof. In a related aspect, the stem or progenitor cells are obtained from a tissue selected from a group comprising: bone marrow, umbilical cord blood, peripheral blood, fetal liver, and adipose tissue, or any combination thereof.

In another aspect, disclosed herein is a pharmaceutical composition comprising a 3D cell cluster comprising transdifferentiated mammalian non-pancreatic beta insulin producing cells and a polysaccharide matrix, wherein at least a subset of said cells are attached to said polysaccharide matrix.

In another aspect, disclosed herein is a method of generating a 3D cell cluster comprising transdifferentiated mammalian non-pancreatic beta insulin producing cells and a polysaccharide matrix, wherein at least a subset of the cells are attached to said polysaccharide matrix, the method comprising: (a) providing the polysaccharide matrix; (b) obtaining a human tissue; (c) processing said tissue to recover primary human non-pancreatic cells; (d) propagating and expanding the cells of step (c) to a predetermined number of cells; (e) transdifferentiating the cells of step (d); and (f) attaching at least a subset of said cells to said polysaccharide matrix; thereby generating a 3D cell cluster comprising transdifferentiated mammalian non-pancreatic beta insulin producing cells, wherein at least a subset of said cells are attached to a polysaccharide matrix.

In a related aspect, at step (e) the transdifferentiating comprises: (a) infecting said expanded cells with an adenoviral vector comprising a nucleic acid encoding a human PDX-1 polypeptide, said infecting occurring at a first timepoint; (b) infecting said expanded cells of step (a) with an adenoviral vector comprising a nucleic acid encoding a second human pancreatic transcription factor polypeptide, said infecting occurring at a second timepoint; and (c) infecting said expanded cells of step (b) with an adenoviral vector comprising a nucleic acid encoding a human MafA polypeptide, said infecting occurring at a third timepoint.

In a related aspect, the second pancreatic transcription factor is selected from NeuroD1 and Pax4. In a related aspect, the first timepoint and said second timepoint are concurrent. In a related aspect, step (d), step (e), or a combination thereof are executed under non-adherent cell culture conditions.

In one aspect, disclosed herein is a method for treating a pancreatic disease or disorder in a subject, the method comprising administering a 3D cell cluster comprising transdifferentiated mammalian non-pancreatic beta insulin producing cells and a polysaccharide matrix, or a composition comprising said 3D cell cluster, to said subject; thereby treating said disease in said subject.

In a related aspect, administering comprises intradermal, intraperitoneal, or surgical administration, or any combination thereof, of the 3D cell cluster to said subject. In a related aspect, the pancreatic disease or disease comprises type I diabetes, type II diabetes, gestational diabetes, pancreatic cancer, hyperglycemia, pancreatitis, pancreatic pseudocysts, pancreatic trauma caused by injury, or a disease caused by pancreatectomy, or any combination thereof.

### BRIEF DESCRIPTION OF THE DRAWINGS

FIG. 1 shows the chemical formula of alginate.

FIG. 2 shows the chemical formula of alginate sulfate.

FIG. 3 shows the fabrication process of an alginate/alginate sulfate scaffold with affinity-bound TGF-β and growth factors, and loaded with cells.

FIG. 4 shows an overview of one embodiment of the three-dimensional (3D) cell cluster manufacturing process, including its attachment to a scaffold. Steps include: Step 1—Obtaining liver tissue (e.g., a liver biopsy); Step 2—Processing of the tissue to recover primary liver cells; Step 3—Propagating the primary liver cells to predetermined cell number; Step 4—Transdifferentiation of the primary liver cells; and Step 5—Testing the transdifferentiated cells for quality assurance and quality control (i.e., safety, purity and potency). Cells are attached to a scaffold prior to steps 3, 4, or 5, or during step 3. Optional steps include cryopreserving early passage primary liver cells; enriching or sorting transdifferentiation-predisposed liver cells; thawing cryopreserved cells for use at a later date; dissociating single cells from the 3D cluster; and storage of transdifferentiated cells for use at a later date.

FIG. 5 shows an overview of one embodiment of a method for manufacturing an alginate scaffold loaded with bioactive peptides and cells. The starting materials are alginate, the bioactive peptides of interest, and mammalian non-pancreatic beta cells. Steps include: Step 1—crosslinking and freezing the alginate; Step 2—binding the bioactive peptides; and Step 3—attaching mammalian non-pancreatic beta cells to the scaffold. Cells can be attached to the scaffold before, during, or after the bioactive peptides are attached to the scaffold.

FIGS. 6A-6H show the expression of ectopic genes in transdifferentiated insulin producing cells (IPCs) grown in a scaffold, as revealed by confocal microscopy, either 4 or 72 hours after seeding onto the scaffold. FIGS. 6A-6D show confocal images of transdifferentiated IPCs 4 h after seeding, stained for DAPI (FIG. 6A), F-actin (FIG. 6B), and PDX-1 (FIG. 6C). FIG. 6D is a superposition of FIGS. 6A-6C. FIGS. 6E-6H show confocal images of transdifferentiated IPCs 72 h after seeding, stained for DAPI (FIG. 6E), F-actin (FIG. 6F), and PDX-1 (FIG. 6G). FIG. 6H is a superposition of FIGS. 6E-6G. Size bar indicates 50 μm in FIGS. 6A-6D and 200 μm in FIGS. 6E-6H.

FIGS. 7A-7F show the morphology of transdifferentiated insulin producing cells (IPCs) grown in scaffolds and in 6 well plates as revealed by light microscopy. FIGS. 7A-7D show images of IPCs seeded in scaffolds (2.5×10⁶ cells/scaffold) after 4 h (FIGS. 7A and 7B) and after 72 h (FIGS. 7C and 7D). FIGS. 7E and 7F show images of IPCs seeded in 6 well plates (0.5×10⁶ cells/well) after 4 h (FIG. 7E) and after 72 h (FIG. 7F). Size bar indicates 100 μm, using 10× magnification.

FIGS. 8A-8F show the formation of cell clusters by IPCs seeded on scaffolds. 0.5×10⁶ (FIGS. 8A and 8B), 1×10⁶ (FIGS. 8C and 8D), and 2.5×10⁶ (FIGS. 8E and 8F) IPCs were seeded on scaffolds. Light microscopy images were taken immediately (FIGS. 8A, 8C, and 8E) and 24 h (FIGS. 8B, 8D, and 8F) after seeding. No cell clusters were observed immediately after seeding (FIGS. 8A, 8C, and 8E). However, clusters were formed after 24 h. Cluster size correlated with the number of cells seeded (FIGS. 8B, 8D, and 8F). 10× magnification was used.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

In the present disclosure the singular forms “a,” “an,” and “the” include the plural reference, and reference to a particular numerical value includes at least that particular value, unless the context clearly indicates otherwise. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable. In some embodiments, the term “about”, refers to a deviance of between 0.0001-5% from the indicated number or range of numbers. In some embodiments, the term “about”, refers to a deviance of between 1-10% from the indicated number or range of numbers. In some embodiments, the term “about”, refers to a deviance of up to 25% from the indicated number or range of numbers. The term “comprises” means encompasses all the elements listed, but may also include additional, unnamed elements, and it may be used interchangeably with the terms “encompasses”, “includes”, or “contains” having all the same qualities and meanings. The term “consisting of” means being composed of the recited elements or steps, and it may be used interchangeably with the terms “composed of” having all the same qualities and meanings.

The disclosure relates to compositions and methods for providing transdifferentiated cells in scaffolds to treat pancreatic, liver, and other diseases. Further disclosed herein are three-dimensional (3D) cell clusters comprising transdifferentiated insulin producing cells, wherein said clusters are attached to a scaffold. In some embodiments, transdifferentiated cells are capable of producing and secreting pancreatic hormones. In some embodiments, said cells are encapsulated within said scaffold. Further disclosed herein are methods for producing 3D cell clusters of transdifferentiated cells attached to scaffolds. Further disclosed herein are methods for treating a pancreatic disorder, the method comprising administering a 3D cell cluster of transdifferentiated cells attached to a scaffold to a subject in need thereof.

Three-Dimensional (3D) Cell Clusters

In some embodiments, disclosed herein is a 3D cell cluster comprising transdifferentiated mammalian non-pancreatic beta insulin producing cells, wherein at least a subset of said cells are attached to a scaffold. A skilled artisan would appreciate that the term “3D cell cluster” may encompass a group of cells physically contacting each other and organized in a 3D structure. A cell in a 3D cluster can contact other cells located in any direction relative to itself (i.e., above, below and on the laterals). A 3D cluster may be suspended in a culture medium, having its entire external surface contacting the medium. This contrasts with 2D cell clusters or other types of monolayer cell cultures. A cell in a 2D cluster is attached to the plate on one of its sides, and can only contact other cells located on its laterals. Similarly, only one side of 2D cluster can be in physical contact with the medium. The term “3D cell cluster” may be used interchangeably with “cell spheroid”, “multicell spheroid”, “3D cell colonies” having all the same qualities and meanings.

In some embodiments, the size of the 3D cell cluster is smaller than 10 μm. In some embodiments, the size of the 3D cell cluster is between 10 μm to 50 μm. In some embodiments, the size of the 3D cell cluster is between 50 μm to 100 μm. In some embodiments, the size of the 3D cell cluster is between 100 μm to 200 μm. In some embodiments, the size of the 3D cell cluster is between 200 μm to 300 μm. In some embodiments, the size of the 3D cell cluster is between 300 μm to 400 μm. In some embodiments, the size of the 3D cell cluster is between 400 μm to 500 μm. In some embodiments, the size of the 3D cell cluster is between 500 μm to 600 μm. In some embodiments, the size of the 3D cell cluster is between 600 μm to 700 μm. In some embodiments, the size of the 3D cell cluster is between 700 μm to 800 μm. In some embodiments, the size of the 3D cell cluster is between 800 μm to 900 μm. In some embodiments, the size of the 3D cell cluster is between 900 μm to 1000 μm. In some embodiments, the size of the 3D cell cluster is larger than 1000 μm. In some embodiments, the size of the 3D cluster comprises a maximum diameter. In some embodiments, the size comprises a maximum length. In some embodiments, the size encompasses a minimum diameter. In some embodiments, the size encompasses a volume.

In some embodiments, a 3D cell cluster comprises less than 50 cells. In some embodiments, a 3D cell cluster comprises between about 50 and 500 cells. In some embodiments, a 3D cell cluster comprises between about 500 and 1000 cells. In some embodiments, a 3D cell cluster comprises between about 1000 and 2000 cells. In some embodiments, a 3D cell cluster comprises between about 2000 and 3000 cells. In some embodiments, a 3D cell cluster comprises between about 3000 and 4000 cells. In some embodiments, a 3D cell cluster comprises between about 4000 and 5000 cells. In some embodiments, a 3D cell cluster comprises more than 5000 cells.

In some embodiments, a 3D cell cluster comprises homogeneous cells. In some embodiments, a 3D cell cluster comprises heterogeneous cells. In some embodiments, a 3D cell cluster comprises cells comprising a similar phenotype. In some embodiments, a 3D cell cluster comprises cells comprising different phenotypes.

Scaffolds

In some embodiments, a subset of the transdifferentiated mammalian non-pancreatic beta insulin producing cells (IPC) are attached to a scaffold. In some embodiments, a subset of the cells comprises less than 10% of the cells. In some embodiments, a subset of the cells comprises between about 10% to 20% of the cells. In some embodiments, a subset of the cells comprises between about 20% to 30% of the cells. In some embodiments, a subset of the cells comprises between about 30% to 40% of the cells. In some embodiments, a subset of the cells comprises between about 40% to 50% of the cells. In some embodiments, a subset of the cells comprises between about 50% to 60% of the cells. In some embodiments, a subset of the cells comprises between about 60% to 70% of the cells. In some embodiments, a subset of the cells comprises between about 70% to 80% of the cells. In some embodiments, a subset of the cells comprises between about 80% to 90% of the cells. In some embodiments, a subset of the cells comprises between about 90% to 100% of the cells.

A skilled artisan would appreciate that the term “scaffold” encompasses an object providing structural support for cell attachment. Scaffolds are well known in the art and described, for example, in U.S. Pat. Nos. 6,379,962, 6,143,293, and US patent publications US20070014772, US20150051148, US20100247652, US20100145470, US20050003010, US20140147452, US20070111310, US20070081976, US20030078672, US20170290954, US20170096500, US20160354474, US20150352144, US20090239298, and US20020001610; International Application publications WO2017118979, and WO1995035073; and Shapiro et al., (1997) Biomaterials, 18(8):583-90, Freeman et al., (2009) Biomaterials 30(11):2122-31 and Freeman et al. (2008) Biomaterials 29(22):3260-8, which are each incorporated in their entirety herein by reference.

In some embodiments, the scaffold mimics the natural extracellular environment of the islets. In some embodiments, the scaffold provides resistance to hydrolytic or enzymatic degradation. In some embodiments, the scaffold mimics the hierarchical structure of the human pancreatic islets. In some embodiments, the scaffold encapsulates the cells in immune-protective biomaterials thus enhancing the transplant integration in the host. In some embodiments, scaffold porosity is tuned to promote oxygen and nutrient exchange, while preventing the entry of inflammatory cells and antibodies.

A skilled artisan would appreciate that the term “cell attachment” comprises the physical interaction of a cell to a surface, substrate or another cell, mediated by interaction of molecules of the cell surface, as cell adhesion molecules, selectins, integrins, syndecans, and cadherins. The term “cell attachment” may be used interchangeably with “cell adhesion”, “cell binding”, “cell loading”, and “cell association” having all the same qualities and meanings. In some embodiments, seeding a cell on a surface comprises attaching the cell to that surface. In some embodiments, cell attachment to a scaffold comprises non-covalent forces. In some embodiments, cells are covalently attached to a scaffold.

A skilled artisan would appreciate that the physico-mechanical, biochemical and functional characteristics of a scaffold can be assessed and optimized. The relevant physico-mechanical properties of the scaffold (e.g. elasticity, compressibility, viscoelastic behavior, tensile strength) can be studied, such as the mechanical properties which are influencing the cell adhesion and proliferation. The stability of the scaffolds under physiological conditions can be also assessed. For this purpose, the degradation of the scaffolds can be studied by exposing them to a combination of factors mimicking their natural environment in the site of transplantation (pH, enzymes, temperature, etc.). In vitro cell culture experiments can be performed to evaluate biocompatibility, cell attachment, cell viability and cell proliferation. Experiments can be performed to evaluate cell morphology by using contrast microscopy, cell recovery, and cell viability by using Trypan blue exclusion assay. Experiments can be performed to evaluate cell functionality at the molecular level, including assessing expression of pTF and hormones by real time PCR. Experiments can be performed to evaluate cell functionality at the cellular level, including assessing insulin content by dithizone staining, insulin secretion and content by assessment of C-peptide level by ELISA, and Glucose Stimulated Insulin Secretion (GSIS).

A skilled artisan would appreciate that the immunological profile of a scaffold can be assessed and optimized. Immunogenicity can be tested, for example by exposing peripheral blood mononuclear cells (PBMC) to the scaffold with or without transdifferentiated cells and measuring cytokines and T cell proliferation. Release of cytokines, as IFNγ, can be assessed by collecting PBMC supernatants following 48 hours and measuring cytokines by using commercially available kits. Proliferation of T cells can be assessed by Carboxyfluorescein succinimidyl ester (CFSE) staining following five days of co-incubation. CFSE labeling is diluted with each cell division and therefore it can be used to evaluate proliferations of T cells with flow cytometry. T cell subsets (CD8, CD4, T cells) can be labeled prior to the analysis. In vivo results can be validated by transplanting animals with the scaffold loaded with transdifferentiated cells or with the scaffold alone. In these in vivo experiments, mice are sacrificed at indicated time points post-transplantation and at each time point the transplant is retrieved. Half of the retrieved transplants are cultured, stained and observed under light and fluorescence microscopes to evaluate cell morphology, viability and tissue overgrowth. The other half of the retrieved microcapsules are used for histological analyses for identify reactive CD8 T cells.

A skilled artisan would appreciate that the effects of cell storage, package and transport on viability and function of transdifferentiated insulin producing cells attached to scaffolds can be assessed and optimized. Current methods for islets preservation are based on cold storage at 4° C. and allow for a limited viability of the cells of only 24-48 hours. The functionality of scaffold transdifferentiated cells can be tested at different temperatures and preservation media. Cell viability, gene expression and cell potency at several time points with or without the scaffold can be measured. Functional activity and potency at the end of the stability phase can be considered successful if they do not fall under 70% of the values achieved with the control product. Cells from at least three different donors can be tested. Two formulation solution candidates of transporting media can be used for comparison on the batches generated. The effect of Packaging material (mainly bags) can be established in terms of time, temperature, final cell density, and optimal application volume.

In some embodiments, the scaffold is a solid scaffold. In some embodiments, the scaffold comprises a hydrogel. In some embodiments, the scaffold comprises an extracellular matrix. In some embodiments, the scaffold comprises an extracellular matrix hydrogel. In some embodiments, the scaffold comprises a protein hydrogel. In some embodiments, the scaffold comprises a peptide hydrogel. In some embodiments, the scaffold comprises a polymer hydrogel. In some embodiments, the scaffold comprises a wood-based nanocellulose hydrogel. In some embodiments, the scaffold comprises a polysaccharide matrix. In some embodiments, the scaffold comprises a sulfated polysaccharide matrix. In some embodiments, the scaffold comprises a mixed polysaccharide and sulfated polysaccharide matrix. In some embodiments the scaffold is flexible and amenable to be fixed in place preventing its migration to an unintended location. In some embodiments, the scaffold encapsulates the cells. In some embodiments, the scaffold with the cells are encapsulated in an encapsulation agent.

In some embodiments, the terms “scaffold” and “polysaccharide matrix” are used herein interchangeably, having all the same qualities and meanings. In some embodiments, the terms “scaffold”, “sulfated polysaccharides and polysaccharides matrix”, “mixed polysaccharide and sulfated polysaccharide matrix” are used herein interchangeably, having all the same qualities and meanings.

In some embodiments, the cells attached to a scaffold are cells of the same type. In some embodiments, more than one type of cells is attached to a scaffold. In some embodiments, two types of cells are attached to a scaffold. In some embodiments, three types of cells are attached to a scaffold. In some embodiments, four types of cells are attached to a scaffold. In some embodiments, more than four types of cells are attached to a scaffold.

In some embodiments, the cells attached to a scaffold are transdifferentiated insulin producing cells. In some embodiments, the cells attached to a scaffold are insulin producing cells and lymphocytes. In some embodiments, the cells attached to a scaffold are insulin producing cells and peripheral mononuclear blood cells (PBMC). In some embodiments, the cells attached to a scaffold are insulin producing cells, lymphocytes, and PBMC. In some embodiments, the cells attached to the scaffold comprise transdifferentiated insulin producing cells, mesenchymal stem cells (MSC), endothelial progenitor cells (EPC), or any combination thereof.

In some embodiments, a type of cell attached to the scaffold provides supportive functions to transdifferentiated insulin producing cells. In some embodiments, a type of cells attached to the scaffold generates an immunotolerant environment. In some embodiments, an immunotolerant environment facilitates grafting and survival of the transplanted cells.

A skilled artisan would appreciate that the term “cell type” or “type of cell” comprises a classification used to distinguish between morphologically or phenotypically distinct cell forms. Some non-limiting examples of cell types comprise: epithelial cells, endothelial cells, keratinocytes, fibroblasts, muscle cells, hepatocytes, liver cells, blood cells, stem or progenitor cells, embryonic heart muscle cells, liver stem cells, neural stem cells, mesenchymal stem cells, hematopoietic stem and progenitor cells, insulin producing cells, transdifferentiated insulin producing cells, transdifferentiated cells having a pancreatic beta cell phenotype, transdifferentiated liver cells having a pancreatic beta cell phenotype, lymphocytes, PBMC, pancreatic cells other than pancreatic beta cells, acinar cells, alpha-cells.

Alginate-Based Scaffolds

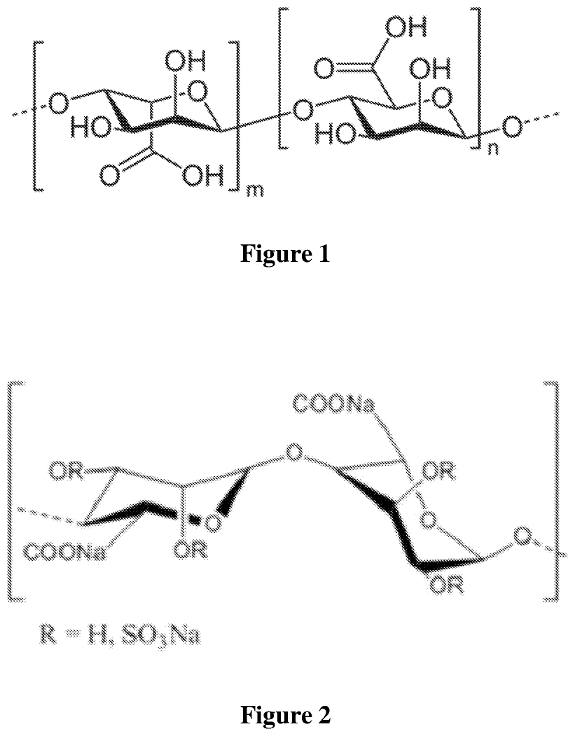

In some embodiments, the scaffold comprises a polysaccharide matrix. In some embodiments, a polysaccharide matrix comprises an alginate polysaccharide. Alginate comprises a linear polysaccharide comprising of β-D-mannuronate and α-L-glucuronate. Alginate has been widely used in tissue engineering. The structural formula of alginate is given in FIG. 1. Alginate-based hydrogels have been shown to provide support for different cell types, including osteoblasts, chondrocytes, fibroblasts, and embryonic stem cells. Alginate based scaffolds are known in the field, as well as their structure and the methods of manufacturing them.

In some embodiments, the term “alginate” refers to a polyanionic polysaccharide copolymer derived from sea algae (e.g., Laminaria hyperborea, L. digitata, Eclonia maxima, Macrocystis pyrifera, Lessonia nigrescens, Ascophyllum codosum, L. japonica, Durvillaea antarctica, and D. potatorum) which includes β-D-mannuronic (M) and a-L-guluronic acid (G) residues in varying proportions.

Alginate scaffolds are hydrophilic in nature, permitting their rapid wetting by aqueous media and efficient cell seeding. Alginate scaffolds are highly porous. In some embodiments, less than around 5% of an alginate scaffold volume is solid or semi-solid. In some embodiments, only around 5% of an alginate scaffold volume is solid or semi-solid. In some embodiments, only around 10% of an alginate scaffold volume is solid or semi-solid. In some embodiments, only around 15% of an alginate scaffold volume is solid or semi-solid. In some embodiments, only around 20% of an alginate scaffold volume is solid or semi-solid. In some embodiments, more than around 20% of an alginate scaffold volume is solid or semi-solid.

In some embodiments, the diameter of alginate scaffold pores is less than 20 μm. In some embodiments, the diameter of alginate scaffold pores is between about 20 μm and about 40 μm. In some embodiments, the diameter of alginate scaffold pores is between about 40 μm and about 60 μm. In some embodiments, the diameter of alginate scaffold pores is between about 60 μm and about 80 μm. In some embodiments, the diameter of alginate scaffold pores is between about 80 μm and about 100 μm. In some embodiments, the diameter of alginate scaffold pores is between about 100 μm and about 120 μm. In some embodiments, the diameter of alginate scaffold pores is between about 120 μm and about 140 μm. In some embodiments, the diameter of alginate scaffold pores is between about 140 μm and about 160 μm. In some embodiments, the diameter of alginate scaffold pores is between about 160 μm and about 180 μm. In some embodiments, the diameter of alginate scaffold pores is between about 180 μm and about 200 μm. In some embodiments, the diameter of alginate scaffold pores is between about 200 μm and about 250 μm. In some embodiments, the diameter of alginate scaffold pores is between about 250 μm and about 300 μm. In some embodiments, the diameter of alginate scaffold pores is between about 300 μm and about 350 μm. In some embodiments, the diameter of alginate scaffold pores is between about 350 μm and about 400 μm. In some embodiments, the diameter of alginate scaffold pores is between about 400 μm and about 450 μm. In some embodiments, the diameter of alginate scaffold pores is between about 450 μm and about 500 μm. In some embodiments, the diameter of alginate scaffold pores is more than 500 μm.

The interconnected large pores of alginate scaffolds allow the relatively free movement of cells throughout the pores. Said free movement allows a uniform distribution of cells throughout the scaffold volume. The connectivity of the pores in the alginate scaffolds allows the re-organization of dispersed cells into multicellular aggregates, thus allowing cell-cell interactions. Furthermore, extracellular matrix (ECM) components that are secreted by the seeded cells contribute to the compaction of the cell aggregates into a tissue-like form.

In some embodiments, alginate can be used as a starting material for producing an alginate scaffold. Alginate is a commercially available product that can be obtained from suppliers, for example but not limited to Pronova Biopolymers (Norway), Sigma-Aldrich (USA), ThermoFisher Scientific (USA), FMC Health and Nutrition (Norway), Vega Pharma Ltd (China).

In some embodiments, alginate comprises a linear copolymer comprising guluronic acid monomers and mannuronic acid monomers. In some embodiments, the guluronic acid monomer content is between about 30% and about 90%. In some embodiments, the guluronic acid monomer content is between about 40% and about 80%. In some embodiments, the guluronic acid monomer content is between about 50% and about 75%. In some embodiments, the guluronic acid monomer content is between about 65% and about 75%.

In some embodiments, alginate has a solution viscosity below about 25 cP (1% w/v at 25° C.). In some embodiments, alginate has a solution viscosity between about 25 cP and about 50 cP (1% w/v at 25° C.). In some embodiments, alginate has a solution viscosity between about 50 cP and about 100 cP (1% w/v at 25° C. In some embodiments, alginate has a solution viscosity between about 100 cP and about 200 cP (1% w/v at 25° C.). In some embodiments, alginate has a solution viscosity between about 200 cP and about 300 cP (1% w/v at 25° C.). In some embodiments, alginate has a solution viscosity above 300 cP (1% w/v at 25° C.).

In some embodiments, the alginate used for the preparation of a scaffold is in a solution at a concentration smaller than 0.25% (w/v). In some embodiments, alginate is in a solution at a concentration between 0.25% and 0.5% (w/v). In some embodiments, alginate is in a solution at a concentration between 0.5% and 1% (w/v). In some embodiments, alginate is in a solution at a concentration between 1% and 1.5% (w/v). In some embodiments, alginate is in a solution at a concentration between 1.5% and 2% (w/v). In some embodiments, alginate is in a solution at a concentration between 2% and 2.5% (w/v). In some embodiments, alginate is in a solution at a concentration between 2.5% and 3% (w/v). In some embodiments, alginate is in a solution at a concentration between 3% and 3.5% (w/v). In some embodiments, alginate is in a solution at a concentration between 3.5% and 4% (w/v). In some embodiments, alginate is in a solution at a concentration between 4% and 4.5% (w/v). In some embodiments, alginate is in a solution at a concentration between 4.5% and 5% (w/v). In some embodiments, alginate is in a solution at a concentration larger than 5% (w/v). In some embodiments, alginate is in a solution at a concentration of 1.42% (w/v).

In some embodiments, alginate scaffolds further comprise controlled-release polymeric microspheres, said microspheres are able to secrete soluble factors in a controlled manner. In some embodiments, said soluble factors comprise growth factors, genes or DNA. In some embodiments, said soluble factors comprise pancreatic transcription factors. In some embodiments, said soluble factors comprise genes encoding pancreatic transcription factors.

Microspheres provide a depot for soluble factors and genes, while controlling their presentation in the tissue or graft. Microspheres are incorporated within the alginate scaffolds in order to maximize the soluble factors effect on the entrapped cells. One of the advantages of using controlled-release microspheres is that, if required, the release rate of the soluble factor from the microspheres can be adjusted according to specific needs. In some embodiments, the microspheres are incorporated into the scaffold during scaffold preparation.

Some embodiments for the preparation of alginate scaffolds are described, for example, in Shapiro et al., (1997) Biomaterials, 18(8):583-90, Freeman et al., (2009) Biomaterials 30(11):2122-31 and Freeman et al. (2008) Biomaterials 29(22):3260-8. In some embodiments, the alginate scaffold is prepared by a three-step procedure: first gelation of the alginate with bivalent cations, followed by freezing of the hydrogel and finally lyophilization to produce a porous sponge. The pattern and the extent of sponge porosity, as well as its mechanical properties, can be influenced by the concentration and the type of alginate (guluronic to mannuronic ratio and viscosity), the type and concentration of the cross-linkers and the freezing regime.

In some embodiments, the alginate scaffold is prepared using a pharmaceutical-grade alginate, for example but not limited to Protanal LF 5/60 (Pronova Biopolymers, Drammen, Norway) as starting material. Protanal LF 5/60 has a guluronic acid monomer content of 65-75% and a solution viscosity of 50 cP (1% w/v at 25° C.). In some embodiments, the scaffold preparation consists of (i) preparing sodium alginate stock solutions, at concentrations of 1-3% (w/v), (ii) cross-linking the alginate by adding, dropwise, the bivalent cross-linker, for example but not limited to calcium gluconate, (iii) freezing the cross-linked alginate, and (iv) lyophilizing the frozen alginate to produce a sponge-like scaffold. Sponges can be sterilized using ethylene oxide gas apparatus. The residual ethylene oxide can be removed by aeration of the samples with warm air flow. The sponges can be stored in laminated bags, at room temperature, until use.

In some embodiments, the alginate scaffold is prepared by a freeze-dry technique. In some embodiments, a 1.2% (w/v) sodium alginate solution is cross-linked with a 1.32% (w/v) D-gluconic acid/hemicalcium salt by homogenizing the solution to obtain a homogenous calcium ions distribution. Final component concentrations in the cross-linked solutions can be 1.0% and 0.22% (w/v) for the alginate and for the cross-linker, respectively. Fifty microliters of the cross-linked alginate solution are poured into each well of 96-well plates, cooled to 4° C., frozen at −20° C. for 24 h, and lyophilized for 48 h at 0.08 bar and −57° C. Sterilization of the scaffolds is achieved by exposure to ultra-violet (UV) light in a biological hood for 1 h.

In some embodiments, the final concentration of alginate is below 0.1%. In some embodiments, the final concentration of alginate is between 0.1% and 0.2%. In some embodiments, the final concentration of alginate is between 0.2% and 0.4%. In some embodiments, the final concentration of alginate is between 0.4% and 0.6%. In some embodiments, the final concentration of alginate is between 0.6% and 0.8%. In some embodiments, the final concentration of alginate is between 0.8% and 1%. In some embodiments, the final concentration of alginate is between 1% and 1.2%. In some embodiments, the final concentration of alginate is between 1.2% and 1.4%. In some embodiments, the final concentration of alginate is between 1.4% and 1.6%. In some embodiments, the final concentration of alginate is between 1.6% and 1.8%. In some embodiments, the final concentration of alginate is between 1.8% and 2%. In some embodiments, the final concentration of alginate is higher than 2%.

In some embodiments, the final concentration of alginate is 0.5%. In some embodiments, the final concentration of alginate is 1%. In some embodiments, the final concentration of alginate is 1.5%. In some embodiments, the final concentration of alginate is 2%.

In some embodiments, the final concentration of the cross linker is below 0.01%. In some embodiments, the final concentration of the cross linker is between 0.01% and 0.05%. In some embodiments, the final concentration of the cross linker is between 0.05% and 0.1%. In some embodiments, the final concentration of the cross linker is between 0.15% and 0.2%. In some embodiments, the final concentration of the cross linker is between 0.2% and 0.5%. In some embodiments, the final concentration of the cross linker is between 0.5% and 1%. In some embodiments, the final concentration of the cross linker is between 1% and 2%. In some embodiments, the final concentration of the cross linker is higher than 2%.

In some embodiments, the final concentration of the cross linker is 0.1%. In some embodiments, the final concentration of the cross linker is 0.15%. In some embodiments, the final concentration of the cross linker is 0.16%. In some embodiments, the final concentration of the cross linker is 0.2%.

In some embodiments, the alginate is cross-linked with a cross-linker selected from a group comprising calcium phosphate, calcium chloride, and D-gluconic acid hemicalcium salt. Some embodiments of a method for preparing an alginate scaffold cross-linked with calcium are described, for example, in Cardoso et al. (2014) J Biomed Mater Res 102(3):808-17 and in International Application publication WO 2017175229, which are incorporated herein by reference in their entirety. In some embodiments, the calcium phosphate is amorphous calcium phosphate. In some embodiments, amorphous calcium phosphate serves as a source of calcium for cross-linking the alginate. In some embodiments, amorphous calcium phosphate creates void volumes in the scaffold. In some embodiments, amorphous calcium phosphate causes the scaffold to be porous. In some embodiments, the addition of amorphous calcium phosphate to alginate induces latent cross-linking. In some embodiments, latent cross-linking is a delayed cross-linking. In some embodiments, the delay is due to the dissolving of the cross-linking agent over time before being active. In some embodiments, the time frame for the dissolution of the calcium phosphate is minutes to hours. In one embodiment, cross-linking the scaffold increases its rigidity. In some embodiments, more than one cross-linker can be used in the preparation of the scaffold.

In some embodiments, alginate suitable for preparing alginate scaffolds has a ratio between a-L-guluronic acid and β-D-mannuronic ranging between 1:1 to 3:1. In some embodiments, the ratio between a-L-guluronic acid and β-D-mannuronic ranges between 1.5:1 and 2.5:1. In some embodiments, the ratio between a-L-guluronic acid and β-D-mannuronic is about 2. In some embodiments, alginate suitable for preparing alginate scaffolds has a molecular weight ranging between 1 to 300 kDa. In some embodiments, alginate suitable for preparing alginate scaffolds has a molecular weight ranging between 5 to 200 kDa. In some embodiments, alginate suitable for preparing alginate scaffolds has a molecular weight ranging between 10 to 100 kDa. In some embodiments, alginate suitable for preparing alginate scaffolds has a molecular weight ranging between 20 to 50 kDa.

In some embodiments, peptides are covalently bound to the alginate used as a starting material for producing the scaffold. In some embodiments, peptides bound to alginate promote the interaction of the matrix with the loaded cells. In some embodiments, peptides bound to alginate comprise fibronectin-derived peptides. In some embodiments, a peptide bound to alginate comprises the sequence GGGGRGDY (SEQ ID NO:1). In some embodiments, a peptide bound to alginate comprises the sequence GGGGSPPRRARVTY (SEQ ID NO:2). In some embodiments, more than one type of peptide is bound to alginate. In some embodiments, a peptide comprising the sequence GGGGRGDY and a peptide comprising the sequence GGGGSPPRRARVTY are bound to alginate. In some embodiments, the peptides GGGGRGDY and GGGGSPPRRARVTY enhance cell attachment to the scaffold.

In some embodiments, a non-biological additive can be added to the scaffold. In some embodiments, more than one non-biological additive can be added to the scaffold. In some embodiments, the non-biological additive is hydroxyapatite. In some embodiments, the non-biological additive is calcium phosphate. In some embodiments, the non-biological additive is mannitol beads. In some embodiments, the non-biological additive is magnesium ions.

Scaffolds Comprising a Sulfated Polysaccharide and a Bioactive Polypeptide

In some embodiments, alginate may successfully be used as a scaffold for attaching cells to. Molecules may not effectively attach to alginate scaffold, since molecules may be rapidly released from alginate hydrogel scaffolds. In particular, biological molecules of interest such as but not limited to cytokines and growth factors, with sizes ranging between 5 to 100 kDa, may be rapidly released from alginate scaffolds. Sulfating the polysaccharides endows them with properties which allow binding and controlled release of biological molecules of interest. In some embodiments, both cells and biological molecules are associated with a scaffold described herein.

In some embodiments, the alginate scaffolds disclosed herein comprise sulfated polysaccharides. In some embodiments, the alginate scaffolds disclosed herein comprise a mix of sulfated polysaccharides and unsulfated polysaccharides. In some embodiments, said sulfated polysaccharides comprise sulfated alginate. In some embodiments, said unsulfated polysaccharides comprise alginate. In some embodiments, the proportion of sulfated polysaccharide may range from about 1% to about 40% of the total polysaccharides. In some embodiments, the proportion of sulfated polysaccharide may range from about 3% to about 30% of the total polysaccharides. In some embodiments, the proportion of sulfated polysaccharide may range from about 4% to about 20% of the total polysaccharides. In some embodiments, the proportion of sulfated polysaccharide may range from about 5% to about 10% of the total polysaccharides. In some embodiments, the aforementioned proportions represent percentage by mass. In some embodiments, the aforementioned proportions represent percentage by weight. In some embodiments, the aforementioned proportions represent percentage by volume.

In some embodiments, a sulfated polysaccharide comprises a homopolysaccharide. In some embodiments, a sulfated polysaccharide comprises a heteropolysaccharide. In some embodiments, the sulfated polysaccharides are composed of monosaccharide units of different lengths. In some embodiments, the sulfated polysaccharides have different types of bonds linking the monosaccharide units. In some embodiments, sulfated polysaccharides are linear. In some embodiments, sulfated polysaccharides are branched.

In some embodiments, sulfated polysaccharides comprise uronic acid residues such as D-glucuronic, D-galacturonic, D-mannuronic, L-iduronic, and L-guluronic acids. Examples of polysaccharides comprising uronic acid residues include, but are not limited to, alginic acid salts, sodium alginate, pectin, gums and mucilages from plant sources; and glycosaminoglycans (GAGs) from animal sources including hyaluronic acid (hyaluronan). Sulfated polysaccharides comprising uronic acid can be chemically sulfated or may be naturally sulfated polysaccharides.

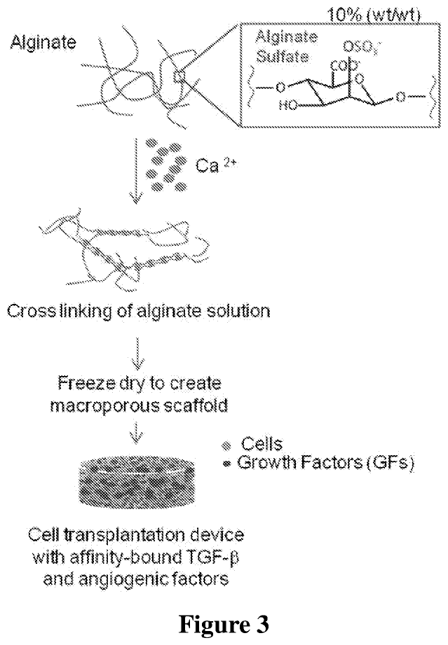

Alginate sulfate and hyaluronan sulfate were both found to mimic the biological specificities of heparan sulfate and heparin when forming bioconjugates and binding bioactive peptides (see for example International Application publication WO 2007/043050, which is hereby incorporated by reference in its entirety). The chemical formula of alginate sulfate is presented in FIG. 2. In some embodiments, the sulfated polysaccharide comprises alginate sulfate. In some embodiments, the sulfated polysaccharide comprises hyaluronan sulfate. In some embodiments, the sulfated polysaccharide is selected from the group comprising: sulfated starch, sulfated glycogen, sulfated cellulose, sulfated chitosan, sulfated chitin, sulfated alginate salts, sulfated hyaluronic acid, sulfated cellulose, sulfated glycogen, and any combination thereof.

Bioactive Polypeptides

In some embodiments, the scaffold comprises a polysaccharide and a first bioactive polypeptide associated with the polysaccharide comprised in the scaffold. In some embodiments, the scaffold comprises a sulfated polysaccharide and a bioactive polypeptide associated to a sulfate group of said sulfated polysaccharide. In some embodiments, the term “bioactive polypeptide” comprises a polypeptide exhibiting a variety of pharmacological activities in vivo and include, without being limited to, growth factors, cytokines, chemokines, angiogenic factors, immunomodulators, hormones, and the like. A skilled artisan would appreciate that the terms “polypeptide” and “proteins” may be used interchangeably having all the same qualities and meanings.

In some embodiments, a bioactive polypeptide comprises a positively-charged polypeptide. In some embodiments, the term “positively charged polypeptide” refers to a polypeptide/protein that has a positive net charge at physiological pH of about pH=7.5. In some embodiments, a positively charged polypeptide is selected from the groups comprising: insulin, glatiramer acetate (also known as Copolymer 1 or Cop 1), antithrombin III, interferon (IFN)-γ (also known as heparin-binding protein), IGF, somatostatin, erythropoietin, luteinizing hormone-releasing hormone (LH-RH), interleukins (IL), IL-2, IL-6, and any combination thereof. In some embodiments, a bioactive polypeptide comprises a mammal polypeptide. In some embodiments, a bioactive polypeptide comprises a human polypeptide. In some embodiments, a bioactive polypeptide comprises a recombinant polypeptide.

In some embodiments, a bioactive polypeptide comprises a heparin-binding polypeptide. In one embodiment, the term “heparin-binding polypeptide” refers to polypeptides or proteins that have clusters of positively-charged basic amino acids and form ion pairs with specially defined negatively-charged sulfo or carboxyl groups on the heparin chain.

In some embodiments, a heparin-binding polypeptide is selected from a group comprising: thrombopoietin (TPO); proteases/esterases, antithrombin III serine protease inhibitor (SLP1), C1 esterase inhibitor (C1-INH), Vaccinia virus complement control protein (VCP), growth factors, angiogenic factors, fibroblast growth factor (FGF), aFGF, bFGF, a FGF receptor, vascular endothelial growth factor (VEGF), insulin-like growth factor (IGF), hepatocyte growth factor (HGF), transforming growth factor β1 (TGF-β1), a platelet-derived growth factor (PDGF), PDGF-αα, PDGF-ββ, epidermal growth factor (EGF), bone morphogenetic proteins (BMP), BMP-2, BMP-7, chemokines, platelet factor 4 (PF-4, CXCL4), CXCL-12, CXCL-11, stromal cell-derived factor-1 (SDF-I), IL-6, IL-8, IL-4, IL-10, IL-5, IL-13, chemokine (C-C motif) ligand 5 (also CCLS), monocyte chemoattractant protein-1 (MCP-1), macrophage inflammatory peptide-1 (MIP-1), lymphotactin, fractalkine, lipid or membrane-binding proteins, annexin, apolipoprotein E (ApoE), pathogen proteins, human immunodeficiency virus type-1 (HIV-1), coat proteins, HIV-1 gp120, cyclophilin A (CypA), Tat protein, viral coat glycoprotein gC, gB or gD of herpes simplex virus (HSV), an envelope protein of Dengue virus, circumsporozoite (CS) protein of Plasmodium falciparum, bacterial surface adhesion protein OpaA, adhesion proteins, 1-selectin, P-selectin, heparin-binding growth-associated molecule (HB-GAM), thrombospondin type 1 repeat (TSR), peptide myelin oligodendrocyte glycoprotein (MOG), and amyloid P (AP), as well as their fragments, mutants, homologs, analogs and allelic variants.

In some embodiments, a bioactive polypeptide comprises transforming growth factor β (TGF-β). The present disclosure encompasses all the known isoforms of TGF (TGF-β1, TGF-β2, TGF-β3, TGF-β4, and TGF-β5), as well as their fragments, mutants, homologs, analogs and allelic variants. In one embodiment, TGF-β comprises a mammalian TGF-β. In one embodiment, TGF-β comprises TGF-β1. In another embodiment, TGF-β comprises TGF-β2. In another embodiment, TGF-β comprises TGF-β3. In another embodiment, TGF-β comprises TGF-β4. In another embodiment, TGF-β comprises TGF-β5. In some embodiments, TGF-β comprises either human TGF-β1 (Genbank Accession No X02812). In some embodiments, TGF-β comprises mouse TGF-β1 (Genbank Accession No AJ00986).

In some embodiments, a scaffold comprising a TGF-β polypeptide generates an immunotolerant microenvironment. In some embodiments, a scaffold comprising TGF-β induces immune suppression. In some embodiments, TGF-β is released locally, thereby achieving a highly localized immune suppression. In some embodiments, localized suppression of immune responses can prevent the large scale side effects associated with systemic administration of TGF-β. In some embodiments, the immune suppression is achieved through several TGF-β mediated effects. In some embodiments, TGF-β mediated effects comprise inhibition of dendritic cell (DCs) maturation, Tregs increase, reduction of the effector functions of CD4 and CD8 cytotoxic T cells in an IL-10-dependent manner, reduction of pro-inflammatory cytokines levels, or any combination thereof. In some embodiments, the local immunoregulatory effects of TGF-β1 are projected to the spleen, resulting in significantly reduced effector functions of allofibroblast-specific CD4 and CD8 T cells. In some embodiments, localized immune suppression improves allograft success, by reducing or preventing allograft rejection.

In some embodiments, an angiogenic factor comprises a molecule involved in the formation of new blood vessels. In some embodiments, a scaffold comprising angiogenic factors stimulate vascularization of the allogeneic cell transplant. In some embodiments, increased vascularization of the allogeneic cell transplant promotes the integration of said allogeneic cell transplant. In some embodiments, increased vascularization of the allogeneic cell transplant allows higher oxygen consumption by the transplanted cells. In some embodiments, increased vascularization of the allogeneic cell transplant increases survival of the transplanted cells. In some embodiments, increased vascularization of the allogeneic cell transplant enhances the desired functioning of the transplanted cells. In some embodiments, the desired functioning comprises increased insulin production. In some embodiments, the desired functioning comprises acquiring and maintaining a pancreatic beta cell phenotype.

In some embodiments, an angiogenic factor comprises VEGF. In some embodiments, an angiogenic factor comprises PDGF-ββ. In some embodiments, an angiogenic factor comprises VEGFR2. In some embodiments, an angiogenic factor comprises endoglin. In some embodiments, an angiogenic factor comprises CD105. In some embodiments, an angiogenic factor comprises EDG-1. In some embodiments, an angiogenic factor comprises HHT1. In some embodiments, an angiogenic factor comprises ORW. In some embodiments, an angiogenic factor comprises ORW1. In some embodiments, an angiogenic factor comprises TGF beta co-receptor. In some embodiments, an angiogenic factor is selected from the group comprising: Angiogenin, Angiopoietin-1, Del-1, acidic fibroblast growth factor (aFGF), basic fibroblast growth factor (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN), Progranulin, Proliferin, Transforming growth factor-alpha (TGF-alpha), Transforming growth factor-beta (TGF-beta), Tumor necrosis factor-alpha (TNF-alpha), Vascular endothelial growth factor (VEGF), and vascular permeability factor (VPF).

In some embodiments, an angiogenic factor comprises an angiogenic protein. In some embodiments, an angiogenic protein comprises a growth factor. In some embodiment, an angiogenic protein is selected from the group comprising Fibroblast growth factors (FGF), VEGF, VEGFR, Neuropilin 1 (NRP-1), Angiopoietin 1 (Ang 1), Tie2, Platelet-derived growth factor (PDGF, BB-homodimer), PDGFR, Transforming growth factor-beta (TGF-β), endoglin, TGF-β receptors, monocyte chemotactic protein-1 (MCP-1), Integrins αVβ3, αVβ5, a5β1, VE-cadherin, CD31, ephrin, plasminogen activators, plasminogen activator inhibitor-1, Nitric oxide synthase (NOS), COX-2, AC133, and Id1/Id3. In some embodiments, an angiogenic protein comprises an angiopoietin. In some embodiments, angiopoietin is selected from a group comprising Angiopoietin 1, Angiopoietin 3, Angiopoietin 4, and Angiopoietin 6.

In some embodiments, ATIII is associated to a sulfated polysaccharide matrix. In some embodiments, TPO is associated to a sulfated polysaccharide matrix. In some embodiments, SLP1 is associated to a sulfated polysaccharide matrix. In some embodiments, C1-INH is associated to a sulfated polysaccharide matrix. In some embodiments, VCP is associated to a sulfated polysaccharide matrix. In some embodiments, FGF is associated to a sulfated polysaccharide matrix. In some embodiments, FGF receptor is associated to a sulfated polysaccharide matrix. In some embodiments, VEGF is associated to a sulfated polysaccharide matrix. In some embodiments, HGF is associated to a sulfated polysaccharide matrix. In some embodiments, IGF is associated to a sulfated polysaccharide matrix. In some embodiments, PDGF is associated to a sulfated polysaccharide matrix. In some embodiments, BMP is associated to a sulfated polysaccharide matrix. In some embodiments, EGF is associated to a sulfated polysaccharide matrix. In some embodiments, CXCL4 is associated to a sulfated polysaccharide matrix.

In some embodiments, SDF-1 is associated to a sulfated polysaccharide matrix. In some embodiments, IL-6 is associated to a sulfated polysaccharide matrix. In some embodiments, IL-8 is associated to a sulfated polysaccharide matrix. In some embodiments, IL-10 is associated to a sulfated polysaccharide matrix. In some embodiments, RANTES is associated to a sulfated polysaccharide matrix. In some embodiments, MCP-1 is associated to a sulfated polysaccharide matrix. In some embodiments, MIP-1 is associated to a sulfated polysaccharide matrix. In some embodiments, gp120 is associated to a sulfated polysaccharide matrix. In some embodiments, CypA is associated to a sulfated polysaccharide matrix. In some embodiments, Tat protein is associated to a sulfated polysaccharide matrix. In some embodiments, viral coat glycoprotein gC of HSV is associated to a sulfated polysaccharide matrix. In some embodiments, viral coat glycoprotein gB of HSV is associated to a sulfated polysaccharide matrix. In some embodiments, viral coat glycoprotein gD of HSV is associated to a sulfated polysaccharide matrix. In some embodiments, an envelope protein of Dengue virus is associated to a sulfated polysaccharide matrix.

In some embodiments, CS of Plasmodium falciparum is associated to a sulfated polysaccharide matrix. In some embodiments, OpaA is associated to a sulfated polysaccharide matrix. In some embodiments, 1-selectin is associated to a sulfated polysaccharide matrix. In some embodiments, P-selectin is associated to a sulfated polysaccharide matrix. In some embodiments, HB-GAM is associated to a sulfated polysaccharide matrix. In some embodiments, TSR is associated to a sulfated polysaccharide matrix. In some embodiments, MOG is associated to a sulfated polysaccharide matrix. In some embodiments, AP is associated to a sulfated polysaccharide matrix. In some embodiments, TGF-β1 is associated to a sulfated polysaccharide matrix.

In some embodiments, a scaffold comprises a second bioactive polypeptide associated to a sulfate group. In some embodiments, the scaffold comprises a third bioactive polypeptide associated to a sulfate group. In some embodiments, the scaffold comprises more than three different bioactive polypeptides associated to a sulfate group. In some embodiments, a scaffold comprises both VEGF and PDGF-ββ associated to sulfate groups. In some embodiments, a scaffold comprises VEGF, PDGF-ββ, and TGF-β1 associated to sulfate groups.

In some embodiments, a scaffold comprising a MOG polypeptide induces immune tolerogenic effects in CD4+ T cells specific to MOG. Several autoimmunogenic MOG polypeptides are known in the art, and include peptides corresponding to mouse MOG amino acids 1-22, 35-55, and 64-96 (see for example US Patent Publication 2009/0053249, hereby incorporated by reference in its entirety). In some embodiments, the MOG polypeptide comprises the sequence MEVGWYRSPFSRV-VHLYRNGK (mouse MOG35-55). In some embodiments, induction of immune tolerogenic effects in CD4+ T cells prevents autoimmune destruction of pancreatic β cells. MOG is a glycoprotein involved in myelination of nerves. MOG and antibodies against MOG may have a role in autoimmune diseases of the central nervous system, as demyelinating diseases.

In some embodiments, a bioactive peptide non-covalently associates with a sulfate group of the sulfated polysaccharide. A skilled artisan would appreciate that by having a positive charge, a bioactive polypeptide may be reversibly and non-covalently bound to a sulfated polysaccharide, which carry a negative charge due to their sulfur group. A skilled artisan would appreciate that the terms “bond” and “association” comprise a lasting attraction between atoms, ions or molecules. The term “bond” may be used interchangeably with “association” having all the same qualities and meanings. Examples of non-covalent bonds or associations comprise, but are not limited to, ionic bonds, electrostatic interactions, hydrophobic interactions, hydrogen bonds or van der Waals forces. In some embodiments, the binding and release of a bioactive polypeptide can be controlled by the degree of polysaccharide sulfation in the scaffold. In some embodiments, the binding and release of a bioactive polypeptide can be controlled by the extent of sulfated polysaccharide sulfate incorporation into the delivery system. In some embodiments, a bioactive peptide covalently associates with a sulfate group of the sulfated polysaccharide.

In some embodiments, a bioactive peptide associates non-covalently with a scaffold component. In some embodiments, a bioactive peptide associates covalently with a scaffold component.

In some embodiments, non-covalent association of bioactive polypeptides to the scaffold leads to a gradual release of said bioactive polypeptides, which can be sustained over a prolonged period of time. The scaffolds disclosed herein are capable of sustainably release polypeptides over a period of time. In some embodiments, the period of time is of about 10 days post administration. In some embodiments, the period of time is of about 15 days. In some embodiments, the period of time is of about 30 days. In some embodiments, the period of time is of about 60 days. In some embodiments, the period of time is of more than 60 days. In some embodiments, the period of time ranges from about 10 days to about 15 days, or from 10 to 15 days, or from about 15 days to about 30 days, or from 15 to 30 days or from about 30 days to about 60 days, or from 30 to 60 days. In some embodiments, the period of time does not exceed 10 days. In some embodiments, the period of time does not exceed 15 days. In some embodiments, the period of time does not exceed 30 days. In some embodiments, the period of time does not exceed 60 days. In some embodiments, the period of time does not exceed 90 days. In some embodiments, the period of time does not exceed 120 days. In some embodiments, the compositions and methods of the present invention promote systemic release of polypeptides into a subject's bloodstream.

In some embodiments, the concentration of bioactive polypeptides in the alginate is between 1 μg/ml and 10 μg/ml. In some embodiments, the concentration of bioactive polypeptides in the alginate is between 10 μg/ml and 100 μg/ml. In some embodiments, the concentration the bioactive polypeptides of bioactive polypeptides in the alginate is between 100 μg/ml and 1000 μg/ml. In some embodiments, the concentration of bioactive polypeptides in the alginate is between 1 mg/ml and 10 mg/ml.

In some embodiments, the alginate scaffold comprises a multi-compartment hydrogel, wherein each compartment has a different bioactive peptide. Embodiments of scaffolds comprising multi-compartment hydrogels are described, for example, in International Application publication WO2013124855A1 and in Re'em et al. (2012) Acta Biomater 8(9):3283-93.

In some embodiments, a scaffold comprising a multi-compartment hydrogel is prepared according to a method comprising (i) mixing a sulfated polysaccharide and at least one bioactive polypeptide capable of binding said sulfated polysaccharide, thereby forming a bioconjugate; (ii) mixing said bioconjugate of (i) with a material capable of forming a hydrogel, thereby forming a composite material comprising the bioconjugate; (iii) applying said composite material comprising the bioconjugate of (ii) to a scaffold and optionally adding a hydrogel inducer, thereby forming a hydrogel compartment in said scaffold; and (iv) repeating steps (i) to (iii) until the desired number of hydrogel compartments is obtained, wherein each time that step (i) is repeated, the bioconjugate formed in (i) comprises at least one different bioactive peptide and is therefore distinct from the previously obtained bioconjugate, and each new hydrogel compartment formed in (iii) is in contact with and is physically connected to at least one of the previously formed hydrogel compartments. In some embodiments, the hydrogels are constructed using a mold that confers the desired 3D structure.

A skilled artisan will appreciate that the term “bioconjugate” as used herein comprises a sulfated polysaccharide bound covalently or non-covalently to a bioactive polypeptide. A skilled artisan will appreciate that the term “hydrogel” as used herein comprises a network of natural or synthetic hydrophilic polymer chains able to contain water. Non-limiting examples of compounds able to form such networks are alginate, a partially calcium cross-linked alginate solution, chitosan and viscous hyaluronan. A skilled artisan will appreciate that the term “hydrogel inducer” as used herein comprises any compound able to initiate and/or solidify a hydrogel formation. Different hydrogels require different inducers for polymerization. For example, alginate-based hydrogels, in which alginate units are not covalently linked to each other, require a divalent cation, such as Be⁺², Mg⁺² or Ca⁺², preferably Ca⁺², for chelation-based polymerization.

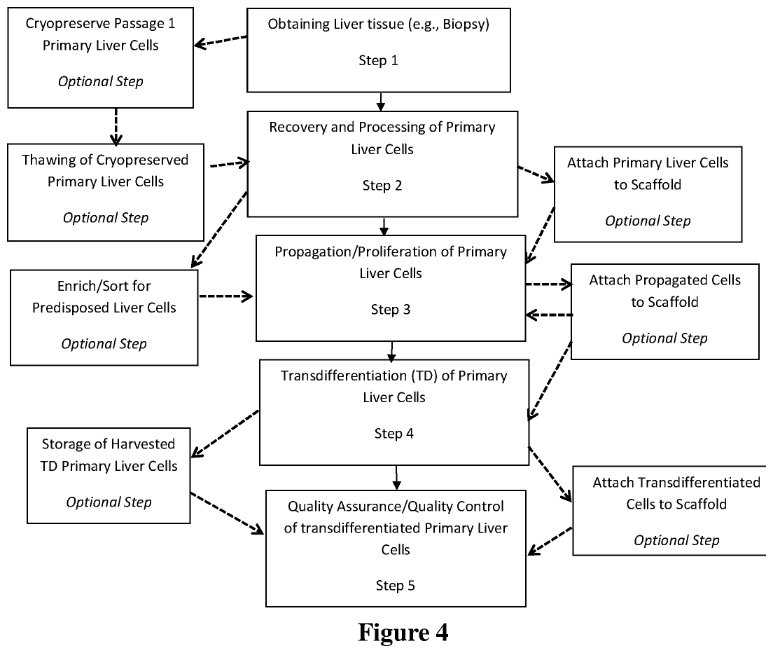

In some embodiments, a bioactive peptide can be bound to the scaffolds comprising sulfated polysaccharides by wetting dry scaffolds in a liquid medium supplemented with said bioactive peptide. FIG. 3 shows a schematic representation of some embodiments of a method for fabricating an alginate/alginate sulfate scaffold loaded with cells, TGF-β1, and growth factors.

In some embodiments, a sulfated alginate scaffold loaded with bioactive peptides is prepared by mixing 0.56 ml 1.42% LVG54 alginate with 0.14 ml 1.42% VLVG alginate. This alginate solution is then mixed with 0.2 ml 0.81% Ca-gluconate (Ca-glu). In parallel, 200 ng of the bioactive peptide(s) of interest are mixed with 50 μl 4.4% of alginate sulfate and incubated at 37° C. 100 μl of the alginate sulfate-protein mix is then mixed with 900 μl of the alginate Ca-glu mix. 50-100 μl of the solution is then poured in 96-plate wells, which are cooled at 4° C. overnight and then at −20° C. overnight. In some embodiments, the final concentration of alginate is 1%, of sulfated alginate 0.2%, and of Ca 0.16%. A skilled artisan would appreciate that in order to produce larger quantities of sulfate alginate scaffold the volumes disclosed herein can be multiplied by any factor.

sodium alginate solution is cross-linked with a 1.32% (w/v) D-gluconic acid/hemicalcium salt by homogenizing the solution to obtain a homogenous calcium ions distribution. Final component concentrations in the cross-linked solutions can be 1.0% and 0.22% (w/v) for the alginate and for the cross-linker, respectively. Fifty microliters of the cross-linked alginate solution are poured into each well of 96-well plates, cooled to 4° C., frozen at −20° C. for 24 h, and lyophilized for 48 h at 0.08 bar and −57° C. Sterilization of the scaffolds is achieved by exposure to ultra-violet (UV) light in a biological hood for 1 h.

Culturing of Mammalian Non-Pancreatic Beta Cells within Alginate Scaffolds

In some embodiments, mammalian non-pancreatic beta cells are seeded within the alginate scaffolds. In some embodiments, cells are seeded at a concentration lower than 0.05×10⁶ cells per scaffold. In some embodiments, cells are seeded at a concentration ranging between 0.05×10⁶ and 0.1×10⁶ cells per scaffold. In some embodiments, cells are seeded at a concentration ranging between 0.1×10⁶ and 0.25×10⁶ cells per scaffold. In some embodiments, cells are seeded at a concentration ranging between 0.25×10⁶ and 0.5×10⁶ cells per scaffold. In some embodiments, cells are seeded at a concentration ranging between 0.5×10⁶ and 1×10⁶ cells per scaffold. In some embodiments, cells are seeded at a concentration ranging between 1×10⁶ and 2.5×10⁶ cells per scaffold. In some embodiments, cells are seeded at a concentration ranging between 2.5×10⁶ and 5×10⁶ cells per scaffold. In some embodiments, cells are seeded at a concentration ranging between 5×10⁶ and 7.5×10⁶ cells per scaffold. In some embodiments, cells are seeded at a concentration ranging between 7.5×10⁶ and 10×10⁶ cells per scaffold. In some embodiments, cells are seeded at a concentration ranging between 10×10⁶ and 20×10⁶ cells per scaffold. In some embodiments, cells are seeded at a concentration higher than 20×10⁶ cells per scaffold.

In some embodiments, cells are seeded at a concentration of about 0.5×10⁶ cells per scaffold. In some embodiments, cells are seeded at a concentration of about 1×10⁶ cells per scaffold. In some embodiments, cells are seeded at a concentration of about 2.5×10⁶ cells per scaffold.

In some embodiments, more than 50% of the seeded cells are efficiently entrapped within the scaffold. In some embodiments, more than 60% of the seeded cells are efficiently entrapped within the scaffold. In some embodiments, more than 70% of the seeded cells are efficiently entrapped within the scaffold. In some embodiments, more than 80% of the seeded cells are efficiently entrapped within the scaffold. In some embodiments, more than 90% of the seeded cells are efficiently entrapped within the scaffold.

In some embodiments, alginate scaffolds have an approximately cylindrical shape. In some embodiments alginate scaffolds have an approximately spheroidal shape. In some embodiments, alginate scaffolds have a diameter smaller than 0.5 mm. In some embodiments, alginate scaffolds have a diameter ranging between about 0.5 mm to 1 mm. In some embodiments, alginate scaffolds have a diameter ranging between about 1 mm to 2.5 mm. In some embodiments, alginate scaffolds have a diameter ranging between about 2.5 mm to 5 mm. In some embodiments, alginate scaffolds have a diameter ranging between about 5 mm to 7.5 mm. In some embodiments, alginate scaffolds have a diameter ranging between about 7.5 mm to 10 mm. In some embodiments, alginate scaffolds have a diameter ranging between about 10 mm to 25 mm. In some embodiments, alginate scaffolds have a diameter ranging between about 25 mm to 50 mm. In some embodiments, alginate scaffolds have a diameter larger than 50 mm.

In some embodiments, alginate scaffolds have a height smaller than 0.1 mm. In some embodiments, alginate scaffolds have a height ranging between about 0.1 mm to 0.25 mm. In some embodiments, alginate scaffolds have a height ranging between about 0.25 mm to 0.5 mm. In some embodiments, alginate scaffolds have a height ranging between about 0.5 mm to 0.75 mm. In some embodiments, alginate scaffolds have a height ranging between about 0.75 mm to 1 mm. In some embodiments, alginate scaffolds have a height ranging between about 1 mm to 2.5 mm. In some embodiments, alginate scaffolds have a height ranging between about 2.5 mm to 5 mm. In some embodiments, alginate scaffolds have a height ranging between about 5 mm to 10 mm. In some embodiments, alginate scaffolds have a height larger than 10 mm. In some embodiments, alginate scaffolds have a cylindrical shape of 5 mm diameter and 1.0 mm height.

In some embodiments, cells are seeded onto the alginate scaffolds by a dynamic method, as the centrifugal packing method. A small volume (50-100 μl) of cell suspension is dropped on top of the scaffold or injected into its center via a 25 G needle Immediately after overlayering the cells, the plate containing the scaffolds is centrifuged using a bench-type centrifuge, at 3000 rpm for 5 min. Due to their hydrophilic nature, the alginate scaffolds are easily wetted by the medium, and an efficient cell seeding can be achieved. In some embodiments, the seeded constructs, supplied with media, are incubated in a humidified atmosphere of 5% CO2 and 95% air, at 37° C.

In some embodiments, the efficiency of cell loading within the scaffold is characterized within 24 hr after cell seeding, for example by determining the total cell number by quantifying the DNA content of a crude cellular homogenate of the cells using the fluorescence enhancement of 4′,6-diamidino-2-phenylindole (DAPI) complexed with DNA, as presently known in the art. In some embodiments, the number of viable cells in the scaffolds is evaluated using the 3-{4,5-dimethylthiazol-2-yl}-2,5-diphenyltetrazolium bromide (MTT) assay, which measures the ability of mitochondrial dehydrogenase enzymes to convert the soluble yellow MTT salt into insoluble purple formazan salt, as presently known in the art. In some embodiments, between 85-90% of the seeded cells are efficiently entrapped within the scaffolds. In some embodiments, the alginate scaffolds are capable of retaining the cells for 1 month without significant cell leakage from the scaffolds.

Encapsulation

In some embodiments, a 3D cell cluster comprising transdifferentiated mammalian non-pancreatic beta insulin producing cells and a scaffold are encapsulated. In some embodiments, the 3D cell cluster is encapsulated in an encapsulation agent. A skilled artisan would appreciate that the term “encapsulation agent” refers to a polymeric semi-permeable membrane that surrounds the cells and selectively permits the bidirectional diffusion of desired molecules, including the influx of molecules essential for cell metabolism and the efflux of molecules of therapeutic value and waste products. In some embodiments, the encapsulation agent protects transdifferentiated cells from immune rejection by the patient. In some embodiments, the encapsulation agent increases transdifferentiated cells viability compared to non-encapsulated transdifferentiated cells. In some embodiments, the encapsulation agent increases insulin secretion from transdifferentiated cells compared to non-encapsulated transdifferentiated cells. A skilled artisan would appreciate that the term “encapsulate” refers to enclosing an object within a membrane. In some embodiments, the membrane comprises a polymer semi-permeable membrane.

In some embodiments, mammalian non-pancreatic beta cells are encapsulated and then attached to a scaffold. In some embodiments, at least part of the mammalian non-pancreatic beta cells are encapsulated within the scaffold. In some embodiments, most mammalian non-pancreatic beta cells are encapsulated within said scaffold. In some embodiments, all mammalian non-pancreatic beta cells are encapsulated within said scaffold. In some embodiments, non-pancreatic beta cells are seeded on a scaffold, and subsequently the scaffold with the cells are encapsulated in an encapsulation agent. In some embodiments, soluble factors are included within the encapsulation agent. In some embodiments, factors promoting cell transdifferentiation are included within the encapsulation agent. In some embodiments, factors promoting cell survival are included within the encapsulation agent. A skilled artisan would appreciate that implantation of transdifferentiated cells encapsulated within semi-permeable membranes presents a number of advantages. First, encapsulated grafts are not rejected by the immune system. Second, encapsulation increases graft survival. Third, encapsulation reduces undesirable side-effects. Fourth, encapsulation reduces the need for long-term use of immunosuppressive drugs. Additionally, encapsulation allows grafts to be retrieved from the patient, in order to follow up cell potency in vivo, or in case the graft is damaging or risking the implanted patient.

In some embodiments, an encapsulating agent comprises alginate. In some embodiments, an encapsulating agent comprises cellulose sulphate. In some embodiments, an encapsulating agent comprises collagen. In some embodiments, an encapsulating agent comprises chitosan. In some embodiments, an encapsulating agent comprises gelatin. In some embodiments, an encapsulating agent comprises agarose. In some embodiments, an encapsulating agent comprises polyethylene glycol (PEG). In some embodiments, an encapsulating agent comprises poly-L-lysine (PLL). In some embodiments, an encapsulating agent comprises polysulphone (PSU). In some embodiments, an encapsulating agent comprises polyvinyl alcohol (PVA). In some embodiments, an encapsulating agent comprises polylactic acid (PLA). In some embodiments, an encapsulating agent comprises acrylates. In some embodiments, an encapsulating agent comprises low molecular weight dextran sulphate (LMW-DS). In some embodiments, an encapsulating agent comprises a derivative of the above disclosed materials. In some embodiments, an encapsulating agent comprises any combination of the above disclosed materials.

Transdifferentiated Cells

A skilled artisan would appreciate that the term “transdifferentiation” may encompass the process by which a first cell type loses identifying characteristics and changes its phenotype to that of a second cell type without going through a stage in which the cells have embryonic characteristics. In some embodiments, the first and second cells are from different tissues or cell lineages. In some embodiments, transdifferentiation involves converting a mature or differentiated cell to a different mature or differentiated cell. Any means known in the art for differentiating or transdifferentiating cells can be utilized. Specifically, lineage-specific transcription factors (TF) have been suggested to display instructive roles in converting adult cells to endocrine pancreatic cells, neurons, hematopoietic cells and cardiomyocyte lineages, suggesting that transdifferentiation processes occur in a wide spectrum of milieus. In all transdifferentiation protocols, ectopic transcription factors serve as a short-term trigger to a potential wide, functional and irreversible developmental process.

In some embodiments, transdifferentiation comprises the differentiation of progenitor cells of pancreatic beta cell lineage, such as pluripotent stem cells, endodermal cells, pancreatic stem cells, endocrine progenitor cells, or progenitors of the endocrine islet lineage. In some embodiments, transdifferentiated non-beta cells comprise insulin producing cells (IPC).

In some embodiments, transdifferentiated mammalian non-pancreatic beta insulin producing cells comprise a pancreatic beta cell phenotype. In some embodiments, a beta cell phenotype comprises the expression of insulin. In some embodiments, a beta cell phenotype comprises the expression of glucagon. In some embodiments, a beta cell phenotype comprises the expression of Nkx6.1, PDX-1, Pax4, Nkx2.2, NeuroD1, Isl1, and Pax6. In some embodiments, transdifferentiated mammalian non-pancreatic beta insulin producing cells comprise a mature pancreatic beta cell phenotype. A skilled artisan would appreciate that, in some embodiments, a mature pancreatic beta cell phenotype comprises the ability of the cells to engage in at least one of the following actions: glucose-sensing (for which the expression of GLUT2 (in mice) and GLUT1 (in humans) is needed), cell excitability (for which the expression of SUR1 and KIR6.2 is needed), insulin processing (for which the expression of PCSK1 and PCSK2 is needed), uptake of zinc into insulin-secretory granules (for which the expression of ZNT8 is needed), and secretion of chromogranin-B (CHGB) and urocortin 3 (UCN3). In some embodiments a mature pancreatic beta cell phenotype comprises the expression of UCN3, ZNT8, MafA, CX36, PSCK1, PSCK2, MafB (in humans), PAX4, NeuroD1, Isl1, Nkx6.1, Glut2, and PDX-1. In some embodiments, a mature pancreatic beta cell phenotype comprises the inactivation of the genes MafB (in mice) and Ngn3.

In some embodiment, a mature pancreatic beta cell phenotype and function comprises expression, production, and/or secretion of pancreatic hormones. Pancreatic hormones can comprise, but are not limited to, insulin, somatostatin, glucagon (GCG), or islet amyloid polypeptide (IAPP). Insulin can be hepatic insulin or serum insulin. In some embodiments, the insulin is a fully process form of insulin capable of promoting glucose utilization, and carbohydrate, fat and protein metabolism. In some embodiments, a mature pancreatic beta cell phenotype and function comprises expression and/or production of pancreatic transcription factors. Pancreatic transcription factors can comprise Pdx1, Ngn3, NeuroD1, Pax4, MafA, NKX6.1, NKX2.2, Hnf1α, Hnf4α, Foxo1, CREB family members, NFAT, FoxM1, Snail and/or Asc-2.

In some embodiments, the pancreatic hormone is in a “prohormone” form. In other embodiments, the pancreatic hormone is in a fully processed biologically active form of the hormone. In other embodiments, the pancreatic hormone is under regulatory control, i.e., secretion of the hormone is under nutritional and hormonal control similar to endogenously produced pancreatic hormones. In some embodiments disclosed herein, the hormone is under the regulatory control of glucose.

The pancreatic beta cell phenotype can be determined for example by measuring pancreatic hormone production, i.e., insulin, somatostatin or glucagon protein mRNA or protein expression. Hormone production can be determined by methods known in the art, i.e. immunoassay, Western blot, receptor binding assays or functionally by the ability to ameliorate hyperglycemia upon implantation in a diabetic host. Insulin secretion can also be measured by, for example, C-peptide processing and secretion. In another embodiment, high-sensitivity assays may be utilized to measure insulin secretion. In another embodiment, high-sensitivity assays comprise an enzyme-linked immunosorbent assay (ELISA), a mesoscale discovery assay (MSD), or an Enzyme-Linked ImmunoSpot assay (ELISpot), or an assay known in the art.

In some embodiments, the cells may be directed to produce and secrete insulin using the methods specified herein. The ability of a cell to produce insulin can be assayed by a variety of methods known to those of ordinary skill in the art. For example, insulin mRNA can be detected by RT-PCR or insulin may be detected by antibodies raised against insulin. In addition, other indicators of pancreatic differentiation include the expression of the genes Isl-1, Pdx-1, Pax-4, Pax-6, and Glut-2. Other phenotypic markers for the identification of islet cells are disclosed in U.S. 2003/0138948, incorporated herein in its entirety.

The pancreatic beta cell phenotype can be determined for example by promoter activation of pancreas-specific genes. Pancreas-specific promoters of particular interest include the promoters for insulin and pancreatic transcription factors, i.e. endogenous PDX-1. Promoter activation can be determined by methods known in the art, for example by luciferase assay, EMSA, or detection of downstream gene expression.

In some embodiments, the pancreatic beta-cell phenotype can also be determined by induction of a pancreatic gene expression profile. A skilled artisan would appreciate that the term “pancreatic gene expression profile” may encompass a profile to include expression of one or more genes that are normally transcriptionally silent in non-endocrine tissues, i.e., a pancreatic transcription factor, pancreatic enzymes or pancreatic hormones. Pancreatic enzymes are, for example, PCSK2 (PC2 or prohormone convertase), PC1/3 (prohormone convertase 1/3), glucokinase, glucose transporter 2 (GLUT-2). Pancreatic-specific transcription factors include, for example, Nkx2.2, Nkx6.1, Pax-4, Pax-6, MafA, NeuroD1, NeuroG3, Ngn3, beta-2, ARX, BRAIN4 and Isl-1.

Induction of the pancreatic gene expression profile can be detected using techniques well known to one of ordinary skill in the art. For example, pancreatic hormone RNA sequences can be detected in, e.g., Northern blot hybridization analyses, amplification-based detection methods such as reverse-transcription based polymerase chain reaction or systemic detection by microarray chip analysis. Alternatively, expression can be also measured at the protein level, i.e., by measuring the levels of polypeptides encoded by the gene. In a specific embodiment PC1/3 gene or protein expression can be determined by its activity in processing prohormones to their active mature form. Such methods are well known in the art and include, e.g., immunoassays based on antibodies to proteins encoded by the genes, or HPLC of the processed prohormones.

In some embodiments, transdifferentiated non-pancreatic beta insulin producing cells cultured as a 3D cluster with a scaffold comprise increased glucose regulated C-peptide secretion compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased glucose regulated C-peptide secretion compared to transdifferentiated non-pancreatic beta insulin producing cells cultured as a 3D cell cluster without a scaffold.

In some embodiments, said increase in glucose regulated C-peptide secretion is less than 10%. In some embodiments, said increase in glucose regulated C-peptide secretion is between about 10% to 100%. In some embodiments, said increase in glucose regulated C-peptide secretion is between about 200% to 300%. In some embodiments, said increase in glucose regulated C-peptide secretion is between about 300% to 400%. In some embodiments, said increase in glucose regulated C-peptide secretion is between about 400% to 500%. In some embodiments, said increase in glucose regulated C-peptide secretion is between about 500% to 600%. In some embodiments, said increase in glucose regulated C-peptide secretion is between about 600% to 700%. In some embodiments, said increase in glucose regulated C-peptide secretion is between about 700% to 800%. In some embodiments, said increase in glucose regulated C-peptide secretion is between about 800% to 900%. In some embodiments, said increase in glucose regulated C-peptide secretion is between about 900% to 1000%. In some embodiments, said increase in glucose regulated C-peptide secretion is between above 1000%.

In some embodiments, transdifferentiated non-pancreatic beta insulin producing cells cultured as a 3D cluster with a scaffold comprise increased glucose regulated insulin secretion compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased glucose regulated insulin secretion compared to transdifferentiated non-pancreatic beta insulin producing cells cultured as a 3D cell cluster without a scaffold.

In some embodiments, said increase in glucose regulated insulin secretion is less than 10%. In some embodiments, said increase in glucose regulated insulin secretion is between about 10% to 100%. In some embodiments, said increase in glucose regulated insulin secretion is between about 200% to 300%. In some embodiments, said increase in glucose regulated insulin secretion is between about 300% to 400%. In some embodiments, said increase in glucose regulated insulin secretion is between about 400% to 500%. In some embodiments, said increase in glucose regulated insulin secretion is between about 500% to 600%. In some embodiments, said increase in glucose regulated insulin secretion is between about 600% to 700%. In some embodiments, said increase in glucose regulated insulin secretion is between about 700% to 800%. In some embodiments, said increase in glucose regulated insulin secretion is between about 800% to 900%. In some embodiments, said increase in glucose regulated insulin secretion is between about 900% to 1000%. In some embodiments, said increase in glucose regulated insulin secretion is between above 1000%.

In some embodiments, transdifferentiated non-pancreatic beta insulin producing cells cultured as a 3D culture with a scaffold comprise increased insulin secretion compared to transdifferentiated non-pancreatic beta insulin producing cells cultured as a monolayer cell culture. In some embodiments, transdifferentiated non-pancreatic beta insulin producing cells cultured as a 3D culture with a scaffold comprise increased insulin secretion compared to transdifferentiated non-pancreatic beta insulin producing cells cultured as a 3D cell cluster without a scaffold.

In some embodiments, transdifferentiated non-pancreatic beta insulin producing cells cultured as a 3D culture with a scaffold comprise increased C-peptide secretion compared to transdifferentiated non-pancreatic beta insulin producing cells cultured as a monolayer cell culture. In some embodiments, transdifferentiated non-pancreatic beta insulin producing cells cultured as a 3D culture with a scaffold comprise increased C-peptide secretion compared to transdifferentiated non-pancreatic beta insulin producing cells cultured as a 3D cell cluster without a scaffold.

In some embodiments, transdifferentiated non-pancreatic beta insulin producing cells cultured as a 3D cluster with a scaffold comprise increased insulin content compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased insulin content compared to transdifferentiated non-pancreatic beta insulin producing cells cultured as a 3D cell cluster without a scaffold.

In some embodiments, transdifferentiated non-pancreatic beta insulin producing cells cultured as a 3D cluster with a scaffold comprise increased expression of GCG compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased expression of GCG compared to transdifferentiated non-pancreatic beta insulin producing cells cultured as a 3D cell cluster without a scaffold.

In some embodiments, said increased expression of GCG is less than 10%. In some embodiments, said increased expression of GCG is between about 10% to 100%. In some embodiments, said increased expression of GCG is between about 200% to 300%. In some embodiments, said increased expression of GCG is between about 300% to 400%. In some embodiments, said increased expression of GCG is between about 400% to 500%. In some embodiments, said increased expression of GCG is between about 500% to 600%. In some embodiments, said increased expression of GCG is between about 600% to 700%. In some embodiments, said increased expression of GCG is between about 700% to 800%. In some embodiments, said increased expression of GCG is between about 800% to 900%. In some embodiments, said increased expression of GCG is between about 900% to 1000%. In some embodiments, said increased expression of GCG is between above 1000%.

In some embodiments, transdifferentiated non-pancreatic beta insulin producing cells cultured as a 3D cluster with a scaffold comprise increased expression of NKX6.1 compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased expression of NKX6.1 compared to transdifferentiated non-pancreatic beta insulin producing cells cultured as a 3D cell cluster without a scaffold.

In some embodiments, said increased expression of NKX6.1 is less than 2-fold. In some embodiments, said increased expression of NKX6.1 is between about 2-fold to 5-fold. In some embodiments, said increased expression of NKX6.1 is between about 5-fold to 10-fold. In some embodiments, said increased expression of NKX6.1 is between about 10-fold to 20-fold. In some embodiments, said increased expression of NKX6.1 is between about 20-fold to 30-fold. In some embodiments, said increased expression of NKX6.1 is between about 30-fold to 40-fold. In some embodiments, said increased expression of NKX6.1 is between about 40-fold to 50-fold. In some embodiments, said increased expression of NKX6.1 is between about 50-fold to 60-fold. In some embodiments, said increased expression of NKX6.1 is between about 60-fold to 70-fold. In some embodiments, said increased expression of NKX6.1 is between about 70-fold to 80-fold. In some embodiments, said increased expression of NKX6.1 is between about 80-fold to 90-fold. In some embodiments, said increased expression of NKX6.1 is between about 90-fold to 100-fold. In some embodiments, said increased expression of NKX6.1 is above 100-fold.

In some embodiments, transdifferentiated non-pancreatic beta insulin producing cells cultured as a 3D cluster with a scaffold comprise increased expression of PAX6 compared to transdifferentiated non-pancreatic beta insulin producing cells cultured as a monolayer cell culture. In some embodiments, transdifferentiated non-pancreatic beta insulin producing cells cultured as a 3D cluster with a scaffold comprise increased expression of PAX6 compared to transdifferentiated non-pancreatic beta insulin producing cells cultured as a 3D cell cluster without a scaffold.

In some embodiments, said increased expression of PAX6 is less than 10%. In some embodiments, said increased expression of PAX6 is between about 10% to 100%. In some embodiments, said increased expression of PAX6 is between about 200% to 300%. In some embodiments, said increased expression of PAX6 is between about 300% to 400%. In some embodiments, said increased expression of PAX6 is between about 400% to 500%. In some embodiments, said increased expression of PAX6 is between about 500% to 600%. In some embodiments, said increased expression of PAX6 is between about 600% to 700%. In some embodiments, said increased expression of PAX6 is between about 700% to 800%. In some embodiments, said increased expression of PAX6 is between about 800% to 900%. In some embodiments, said increased expression of PAX6 is between about 900% to 1000%. In some embodiments, said increased expression of PAX6 is between above 1000%.

In some embodiments, the transdifferentiated non-pancreatic beta insulin producing cells cultured as a 3D cluster with a scaffold secrete at least 20 pm C-peptide/10⁶ cells/hour. In some embodiments, the transdifferentiated non-pancreatic beta insulin producing cells cultured as a 3D cluster with a scaffold secrete at least 50 pm C-peptide/10⁶ cells/hour. In some embodiments, the transdifferentiated non-pancreatic beta insulin producing cells cultured as a 3D cluster with a scaffold secrete at least 100 pm C-peptide/10⁶ cells/hour. In some embodiments, the transdifferentiated non-pancreatic beta insulin producing cells cultured as a 3D cluster with a scaffold secrete at least 200 pm C-peptide/10⁶ cells/hour. In some embodiments, the transdifferentiated non-pancreatic beta insulin producing cells cultured as a 3D cluster with a scaffold secrete at least 500 pm C-peptide/10⁶ cells/hour. In some embodiments, the transdifferentiated non-pancreatic beta insulin producing cells cultured as a 3D cluster with a scaffold secrete at least 1000 pm C-peptide/10⁶ cells/hour.

In some embodiments, glucose regulated insulin secretion comprises at least 0.001 pg insulin/10⁶ cells/hour in response to high glucose concentrations. In another embodiment, glucose regulated insulin secretion comprises at least 0.002 pg insulin/10⁶ cells/hour in response to high glucose concentrations. In another embodiment, glucose regulated insulin secretion comprises at least 0.003 pg insulin/10⁶ cells/hour in response to high glucose concentrations. In another embodiment, glucose regulated insulin secretion comprises at least 0.005 pg insulin/10⁶ cells/hour in response to high glucose concentrations. In another embodiment, glucose regulated insulin secretion comprises at least 0.007 pg insulin/10⁶ cells/hour in response to high glucose concentrations. In another embodiment, glucose regulated insulin secretion comprises at least 0.01 pg insulin/10⁶ cells/hour in response to high glucose concentrations. In another embodiment, glucose regulated insulin secretion comprises at least 0.1 pg insulin/10⁶ cells/hour in response to high glucose concentrations. In another embodiment, glucose regulated insulin secretion comprises at least 0.5 pg insulin/10⁶ cells/hour in response to high glucose concentrations. In another embodiment, glucose regulated insulin secretion comprises at least 1 pg insulin/10⁶ cells/hour in response to high glucose concentrations. In another embodiment, glucose regulated insulin secretion comprises at least 5 pg insulin/10⁶ cells/hour in response to high glucose concentrations. In another embodiment, glucose regulated insulin secretion comprises at least 10 pg insulin/10⁶ cells/hour in response to high glucose concentrations. In another embodiment, glucose regulated insulin secretion comprises at least 50 pg insulin/10⁶ cells/hour in response to high glucose concentrations. In another embodiment, glucose regulated insulin secretion comprises at least 100 pg insulin/10⁶ cells/hour in response to high glucose concentrations. In another embodiment, glucose regulated insulin secretion comprises at least 500 pg insulin/10⁶ cells/hour in response to high glucose concentrations. In another embodiment, glucose regulated insulin secretion comprises at least 1 ng insulin/10⁶ cells/hour in response to high glucose concentrations. In another embodiment, glucose regulated insulin secretion comprises at least 5 ng insulin/10⁶ cells/hour in response to high glucose concentrations. In another embodiment, glucose regulated insulin secretion comprises at least 10 ng insulin/10⁶ cells/hour in response to high glucose concentrations. In another embodiment, glucose regulated insulin secretion comprises at least 50 ng insulin/10⁶ cells/hour in response to high glucose concentrations. In another embodiment, glucose regulated insulin secretion comprises at least 100 ng insulin/10⁶ cells/hour in response to high glucose concentrations. In another embodiment, glucose regulated insulin secretion comprises at least 500 ng insulin/10⁶ cells/hour in response to high glucose concentrations. In another embodiment, glucose regulated insulin secretion comprises at least 1 μg insulin/10⁶ cells/hour in response to high glucose concentrations. In another embodiment, glucose regulated insulin secretion comprises at least 5 μg insulin/10⁶ cells/hour in response to high glucose concentrations. In another embodiment, glucose regulated insulin secretion comprises at least 10 μg insulin/10⁶ cells/hour in response to high glucose concentrations. In another embodiment, glucose regulated insulin secretion comprises at least 50 μg insulin/10⁶ cells/hour in response to high glucose concentrations. In another embodiment, glucose regulated insulin secretion comprises at least 100 μg insulin/10⁶ cells/hour in response to high glucose concentrations.

In some embodiments, the transdifferentiated non-pancreatic beta insulin producing cells cultured as a 3D cluster with a scaffold comprise increased expression of the ectopic pancreatic transcription factors used for transdifferentiation compared to transdifferentiated non-pancreatic beta insulin producing cells transdifferentiated with similar ectopic pancreatic transcription factors and cultured as a monolayer cell culture. In some embodiments, the transdifferentiated non-pancreatic beta insulin producing cells cultured as a 3D cluster with a scaffold comprise increased expression of the ectopic pancreatic transcription factors used for transdifferentiation compared to transdifferentiated non-pancreatic beta insulin producing cells transdifferentiated with similar ectopic pancreatic transcription factors and cultured as a 3D cell cluster without a scaffold. In some embodiments, the ectopic pancreatic transcription factors are selected from PDX1, NeuroD1, Pax4 and/or MafA or any combination thereof.

In some embodiments, the expression of ectopic PDX1 is increased by at least 25% compared to the cells cultured as a monolayer. In some embodiments, said expression is increased by at least 50% compared to the cells cultured as a monolayer. In some embodiments, said expression is increased by at least 100% compared to the cells cultured as a monolayer. In some embodiments, said expression is increased by at least 200% compared to the cells cultured as a monolayer. In some embodiments, said expression is increased by at least 500% compared to the cells cultured as a monolayer. In some embodiments, said expression is increased by at least 1,000% compared to the cells cultured as a monolayer. In some embodiments, said expression is increased by at least 2,000% compared to the cells cultured as a monolayer. In some embodiments, said expression is increased by at least 10,000% compared to the cells cultured as a monolayer.

In some embodiments, the expression of ectopic PDX1 is increased by at least 25% in transdifferentiated non-pancreatic beta insulin producing cells cultured as a 3D cluster with a scaffold compared to transdifferentiated cells cultured as a 3D cell cluster without a scaffold. In some embodiments, said expression is increased by at least 50% compared to the cells cultured as a 3D cell cluster without a scaffold. In some embodiments, said expression is increased by at least 100% compared to the cells cultured as a 3D cell cluster without a scaffold. In some embodiments, said expression is increased by at least 200% compared to the cells cultured as a 3D cell cluster without a scaffold. In some embodiments, said expression is increased by at least 500% compared to the cells cultured as a 3D cell cluster without a scaffold. In some embodiments, said expression is increased by at least 1,000% compared to the cells cultured as a 3D cell cluster without a scaffold. In some embodiments, said expression is increased by at least 2,000% compared to the cells cultured as a 3D cell cluster without a scaffold. In some embodiments, said expression is increased by at least 10,000% compared to the cells cultured as a 3D cell cluster without a scaffold.

In some embodiments, the expression of ectopic NeuroD1 is increased by at least 25% in transdifferentiated non-pancreatic beta insulin producing cells cultured as a 3D cluster with a scaffold compared to transdifferentiated cells cultured as a monolayer. In some embodiments, said expression is increased by at least 50% compared to the cells cultured as a monolayer. In some embodiments, said expression is increased by at least 100% compared to the cells cultured as a monolayer. In some embodiments, said expression is increased by at least 200% compared to the cells cultured as a monolayer. In some embodiments, said expression is increased by at least 500% compared to the cells cultured as a monolayer. In some embodiments, said expression is increased by at least 1,000% compared to the cells cultured as a monolayer. In some embodiments, said expression is increased by at least 2,000% compared to the cells cultured as a monolayer. In some embodiments, said expression is increased by at least 10,000% compared to the cells cultured as a monolayer.

In some embodiments, the expression of ectopic NeuroD1 is increased by at least 25% in transdifferentiated non-pancreatic beta insulin producing cells cultured as a 3D cluster with a scaffold compared to transdifferentiated cells cultured as a 3D cell cluster without a scaffold. In some embodiments, said expression is increased by at least 50% compared to the cells cultured as a 3D cell cluster without a scaffold. In some embodiments, said expression is increased by at least 100% compared to the cells cultured as a 3D cell cluster without a scaffold. In some embodiments, said expression is increased by at least 200% compared to the cells cultured as a 3D cell cluster without a scaffold. In some embodiments, said expression is increased by at least 500% compared to the cells cultured as a 3D cell cluster without a scaffold. In some embodiments, said expression is increased by at least 1,000% compared to the cells cultured as a 3D cell cluster without a scaffold. In some embodiments, said expression is increased by at least 2,000% compared to the cells cultured as a 3D cell cluster without a scaffold. In some embodiments, said expression is increased by at least 10,000% compared to the cells cultured as a 3D cell cluster without a scaffold.

In some embodiments, the expression of ectopic MafA is increased by at least 25% in transdifferentiated non-pancreatic beta insulin producing cells cultured as a 3D cluster with a scaffold compared to transdifferentiated cells cultured as a monolayer. In some embodiments, said expression is increased by at least 50% compared to the cells cultured as a monolayer. In some embodiments, said expression is increased by at least 100% compared to the cells cultured as a monolayer. In some embodiments, said expression is increased by at least 200% compared to the cells cultured as a monolayer. In some embodiments, said expression is increased by at least 500% compared to the cells cultured as a monolayer. In some embodiments, said expression is increased by at least 1,000% compared to the cells cultured as a monolayer. In some embodiments, said expression is increased by at least 2,000% compared to the cells cultured as a monolayer. In some embodiments, said expression is increased by at least 10,000% compared to the cells cultured as a monolayer.

In some embodiments, the expression of ectopic MafA is increased by at least 25% in transdifferentiated non-pancreatic beta insulin producing cells cultured as a 3D cluster with a scaffold compared to transdifferentiated cells cultured as a 3D cell cluster without a scaffold. In some embodiments, said expression is increased by at least 50% compared to the cells cultured as a 3D cell cluster without a scaffold. In some embodiments, said expression is increased by at least 100% compared to the cells cultured as a 3D cell cluster without a scaffold. In some embodiments, said expression is increased by at least 200% compared to the cells cultured as a 3D cell cluster without a scaffold. In some embodiments, said expression is increased by at least 500% compared to the cells cultured as a 3D cell cluster without a scaffold. In some embodiments, said expression is increased by at least 1,000% compared to the cells cultured as a 3D cell cluster without a scaffold. In some embodiments, said expression is increased by at least 2,000% compared to the cells cultured as a 3D cell cluster without a scaffold. In some embodiments, said expression is increased by at least 10,000% compared to the cells cultured as a 3D cell cluster without a scaffold.

A skilled artisan would appreciate that the term “monolayer cell culture” encompasses a type of culture in which no cell is growing on top of another, but all are growing side by side and often touching each other on the same growth surface. The term “monolayer cell culture” may be used interchangeably with “2D cell culture” having all the same qualities and meanings.

In some embodiments, the transdifferentiated cells have increased viability compared to transdifferentiated non-pancreatic beta insulin producing cells cultured as a monolayer cell culture. In some embodiments, the transdifferentiated cells have similar viability than transdifferentiated non-pancreatic beta insulin producing cells cultured as a monolayer cell culture. In some embodiments, the transdifferentiated non-pancreatic beta insulin producing cells cultured as a 3D cluster with a scaffold have increased viability compared to transdifferentiated non-pancreatic beta insulin producing cells cultured as a 3D cell cluster without a scaffold. In some embodiments, the transdifferentiated non-pancreatic beta insulin producing cells cultured as a 3D cluster with a scaffold have similar viability than transdifferentiated non-pancreatic beta insulin producing cells cultured as a 3D cell cluster without a scaffold.

In some embodiments, the adult mammalian non-pancreatic beta cells are adult cells. In some embodiments, the mammalian non-pancreatic beta cells are epithelial cells. In some embodiments, the mammalian non-pancreatic beta cells are endothelial cells. In some embodiments, the mammalian non-pancreatic beta cells are keratinocytes. In some embodiments, the mammalian non-pancreatic beta cells are fibroblasts. In some embodiments, the mammalian non-pancreatic beta cells are muscle cells. In some embodiments, the mammalian non-pancreatic beta cells are hepatocytes. In some embodiments, the mammalian non-pancreatic beta cells are liver cells. In some embodiments, the mammalian non-pancreatic beta cells are blood cells. In some embodiments, the mammalian non-pancreatic beta cells are stem or progenitor cells. In some embodiments, the mammalian non-pancreatic beta cells are embryonic heart muscle cells. In some embodiments, the mammalian non-pancreatic beta cells are liver stem cells. In some embodiments, the mammalian non-pancreatic beta cells are neural stem cells. In some embodiments, the mammalian non-pancreatic beta cells are mesenchymal stem cells. In some embodiments, the mammalian non-pancreatic beta cells are hematopoietic stem or progenitor cells. In some embodiments, the mammalian non-pancreatic beta cells are pancreatic cells other than pancreatic beta cells. In some embodiments, the mammalian non-pancreatic beta cells are acinar cells. In some embodiments, the mammalian non-pancreatic beta cells are alpha-cells. In some embodiments, the mammalian non-pancreatic beta cells are a combination of different cell types.

On one embodiment, the cell is totipotent or pluripotent. In some embodiments, the cell is an induced pluripotent stem cells. In some embodiments, stem or progenitor cells are obtained from bone marrow. In some embodiments, stem or progenitor cells are obtained from umbilical cord blood. In some embodiments, stem or progenitor cells are obtained from peripheral blood. In some embodiments, stem or progenitor cells are obtained from fetal liver. In some embodiments, stem or progenitor cells are obtained from adipose tissue. In some embodiments, stem or progenitor cells are obtained from a combination of tissues.

In some embodiments, the source of a cell population disclosed here is a human source. In another embodiment, the source of a cell population disclosed here in is an autologous human source relative to a subject in need of insulin therapy. In another embodiment, the source of a cell population disclosed here in is an allogeneic human source relative to a subject in need of insulin therapy.

In certain embodiments, the cell is a mesenchymal stem cell, also known as a mesenchymal stromal cell, derived from, liver tissue, adipose tissue, bone marrow, skin, placenta, umbilical cord, Wharton's jelly or cord blood. By “umbilical cord blood” or “cord blood” is meant to refer to blood obtained from a neonate or fetus. In some embodiments, cord blood is obtained from a neonate and refers to blood which is obtained from the umbilical cord or the placenta of newborns. These cells can be obtained according to any conventional method known in the art. MSC are defined by expression of certain cell surface markers including, but not limited to, CD105, CD73 and CD90 and ability to differentiate into multiple lineages including osteoblasts, adipocytes and chondroblasts. MSC can be obtained from tissues by conventional isolation techniques such as plastic adherence, separation using monoclonal antibodies such as STRO-1 or through epithelial cells undergoing an epithelial-mesenchymal transition (EMT).

A skilled artisan would appreciate that the term “adipose tissue-derived mesenchymal stem cells” may encompass undifferentiated adult stem cells isolated from adipose tissue and may also be term “adipose stem cells”, having all the same qualities and meanings. These cells can be obtained according to any conventional method known in the art.

A skilled artisan would appreciate that the term, “placental-derived mesenchymal stem cells” may encompass undifferentiated adult stem cells isolated from placenta and may be referred to herein as “placental stem cells”, having all the same meanings and qualities.

In some embodiments, cell population that is exposed to, i.e., contacted with, the compounds (i.e. PDX-1, Pax-4, MafA, NeuroD1 and/or Sox-9 polypeptides or nucleic acid encoding PDX-1, Pax-4, MafA, NeuroD1 and/or Sox-9 polypeptides) can be any number of cells, i.e., one or more cells, and can be provided in vitro, in vivo, or ex vivo. The cell population that is contacted with the transcription factors can be expanded in vitro prior to being contacted with the transcription factors. The obtained cells produce insulin. These cells can be expanded in vitro by methods known in the art prior to transdifferentiation and maturation along the β-cell lineage, and prior to administration or delivery to a patient or subject in need thereof

Therapeutics Compositions

The herein-described tridimensional (3D) clusters of transdifferentiated cells wherein at least a subset of said cells are attached to a scaffold, when used therapeutically, are referred to herein as “therapeutics”. Methods of administration of therapeutics include, but are not limited to, intradermal, intraperitoneal, or surgical routes. The therapeutics of the disclosure presented herein may be administered by any convenient route, for example by infusion, by bolus injection, by surgical implantation and may be administered together with other biologically-active agents. Administration can be systemic or local, e.g. through portal vein delivery to the liver, or to the pancreas. It may also be desirable to administer the therapeutic locally to the area in need of treatment; this may be achieved by, for example, and not by way of limitation, local infusion during surgery, by injection, by means of a catheter, or by means of an implant.

A skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In some embodiments, suitable dosage ranges of the therapeutics of the disclosure presented herein are generally between 1 million and 2 million transdifferentiated cells. In some embodiments, suitable doses are between 2 million and 5 million transdifferentiated cells. In some embodiments, suitable doses are between 5 million and 10 million transdifferentiated cells. In some embodiments, suitable doses are between 10 million and 25 million transdifferentiated cells. In some embodiments, suitable doses are between 25 million and 50 million transdifferentiated cells. In some embodiments, suitable doses are between 50 million and 100 million transdifferentiated cells. In some embodiments, suitable doses are between 100 million and 200 million transdifferentiated cells. In some embodiments, suitable doses are between 200 million and 300 million transdifferentiated cells. In some embodiments, suitable doses are between 300 million and 400 million transdifferentiated cells. In some embodiments, suitable doses are between 400 million and 500 million transdifferentiated cells. In some embodiments, suitable doses are between 500 million and 600 million transdifferentiated cells. In some embodiments, suitable doses are between 600 million and 700 million transdifferentiated cells. In some embodiments, suitable doses are between 700 million and 800 million transdifferentiated cells. In some embodiments, suitable doses are between 800 million and 900 million transdifferentiated cells. In some embodiments, suitable doses are between 900 million and 1 billion transdifferentiated cells. In some embodiments, suitable doses are between 1 billion and 2 billion transdifferentiated cells. In some embodiments, suitable doses are between 2 billion and 3 billion transdifferentiated cells. In some embodiments, suitable doses are between 3 billion and 4 billion transdifferentiated cells. In some embodiments, suitable doses are between 4 billion and 5 billion transdifferentiated cells.

In some embodiments, the dose is 1-2 billion transdifferentiated cells into a 60-75 kg subject. One skilled in the art would appreciate that effective doses may be extrapolated from dose-response curves derived from in vitro or animal model test systems. In another embodiment, the effective dose may be administered intravenously into the liver portal vein.

Cells may also be cultured ex vivo in the presence of therapeutics of the disclosure presented herein in order to proliferate or to produce a desired effect on or activity in such cells. Treated cells can then be introduced in vivo via the administration routes described herein for therapeutic purposes.

Pharmaceutical Compositions

The herein-described tridimensional (3D) clusters of transdifferentiated cells, wherein at least a subset of said cells are attached to a scaffold, can be incorporated into pharmaceutical compositions suitable for administration. Such compositions typically comprise a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, finger's solutions, dextrose solution, and 5% human serum albumin Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition disclosed here is formulated to be compatible with its intended route of administration. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, polyalcohols such as mannitol, sorbitol or sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the 3D clusters are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

Methods of Generating Three-Dimensional (3D) Cell Clusters

Disclosed herein are methods of generating a 3D cell cluster of transdifferentiated mammalian non-pancreatic beta insulin producing cells, wherein at least a subset of said transdifferentiated cells are attached to a scaffold. In some embodiments, the methods comprise propagating, expanding, transdifferentiating and attaching the cells to a scaffold. In some embodiments, a pancreatic beta cell phenotype comprises a mature pancreatic beta cell phenotype.

In some embodiments, the cells are obtained from a human tissue. In some embodiments, the human tissue is processed to recover primary human non-pancreatic cells. In some embodiments, cells are seeded on a scaffold and propagated and/or expanded on it. In some embodiments, cells are transdifferentiated while being attached to a scaffold. In some embodiments, cells are attached to a scaffold following transdifferentiation. In some embodiments, cells are propagated and/or expanded under non-adherent cell culture conditions. In some embodiments, cells are transdifferentiated under non-adherent conditions.

A skilled artisan would appreciate that the term “non-adherent cell culture conditions” encompasses a type of culture in which single cells or small aggregates of cells are grown while suspended in a liquid medium, and that the term may be used interchangeably with “cell suspension culture” having the same qualities and meanings.

In some embodiments, cells can be grown under non-adherent conditions as a batch culture, i.e., growing in a closed system having a specific volume of agitated medium, with no additions of nutrients or removal of waste products. Batch cultures can be maintained in a recipient such as flasks, conical flasks, or well plates mounted on orbital platform shakers. Alternatively, batch cultures can be maintained in nipple flasks, that alternative expose the cells to the medium and to air. Alternatively, batch cultures can be maintained in spinning cultures, consisting of large bottles containing volumes of medium of about 10 liters that spin around their axis at a predetermined speed and are usually tilted in a predetermined angle. Alternatively, batch cultures can be maintained in stirred cultures, consisting of large culture vessels containing medium into which sterile air is bubbled and/or is agitated by stirrers.

In some embodiments, cells can be grown under non-adherent conditions in continuous culture, i.e., a system in which medium is replaced as to provide cells with nutrients and remove waste. Continuous culture can be closed type, i.e, a system in which the cells are retrieved and added back to the culture. Continuous culture can be open type, i.e., both cells and medium are replaced with fresh medium. Open continuous culture can be carried in a chemostat bioreactor, i.e., a bioreactor to which fresh medium is continuously added, while the present medium is continuously removed at the same rate. Open continuous culture can be carried in a turbidostat, which dynamically adjusts the medium flow rate according to the cell concentration in the medium as determined by medium turbidity. Open continuous culture can be carried in an auxostat, which dynamically adjusts the medium flow rate according to a measurement taken, such as pH, oxygen, ethanol concentrations, sugar concentrations, etc.

In some embodiments, 3D clusters attached to a scaffold can be grown in a bioreactor. A skilled artisan would appreciate that a bioreactor can simulate IPC physiological environment in order to promote cell survival, proliferation, or a pancreatic β cell like phenotype. The physiological environment can comprise parameters as temperature, oxygen concentration, carbon dioxide concentration, or any other relevant biological, chemical or mechanical stimuli. In some instances, the bioreactor comprises one or more small plastic cylindrical chambers with monitored temperature and humidity conditions suitable for growing 3D clusters. The bioreactor can also use bioactive synthetic materials such as polyethylene terephthalate membranes to surround the 3D clusters in a closed environment into which any soluble factors of interest can be provided. The chambers of the bioreactor can rotate as to ensure equal cell growth in all directions.

In some embodiments, at least a subset of the primary cells is attached to a scaffold. In some embodiments, at least a subset of the propagated and expanded cells is attached to a scaffold. In some embodiments, at least a subset of the transdifferentiated cells is attached to a scaffold.

Methods for transdifferentiating cells are described in U.S. Pat. No. 6,774,120, U.S. Publication No. 2005/0090465, U.S. Publication No. 2016/0220616, all the contents of which are incorporated by reference in their entireties. In some embodiments, the methods comprise contacting mammalian non-pancreatic cells with pancreatic transcription factors, such as PDX-1, Pax-4, NeuroD1, and MafA, at specific time points. In some embodiments, the methods comprise contacting a mammalian non-pancreatic cell with PDX-1 at a first timepoint; contacting the cells from the first step with Pax-4 at a second timepoint; and contacting the cells from the second step with MafA at a third timepoint. In some embodiments, the methods comprise contacting a mammalian non-pancreatic cell with PDX-1 at a first timepoint; contacting the cells from the first step with NeuroD1 at a second timepoint; and contacting the cells from the second step with MafA at a third timepoint. In another embodiment, the methods comprise contacting a mammalian non-pancreatic cell with PDX-1 and a second transcription factor at a first timepoint and contacting the cells from the first step with MafA at a second timepoint. In yet a further embodiment, a second transcription factor is selected from NeuroD1 and Pax4. In another embodiment, the transcription factors provided together with PDX-1 comprise Pax-4, NeuroD1, Ngn3, or Sox-9. In another embodiment, the transcription factors provided together with PDX-1 comprises Pax-4. In another embodiment, the transcription factors provided together with PDX-1 comprises NeuroD1. In another embodiment, the transcription factors provided together with PDX-1 comprises Ngn3. In another embodiment, the transcription factors provided together with PDX-1 comprises Sox-9.

In other embodiments, the methods comprise contacting a mammalian non-pancreatic cell with PDX-1 at a first timepoint; contacting the cells from the first step with Ngn3 at a second timepoint; and contacting the cells from the second step with MafA at a third timepoint. In other embodiments, the methods comprise contacting a mammalian non-pancreatic cell with PDX-1 at a first timepoint; contacting the cells from the first step with Sox9 at a second timepoint; and contacting the cells from the second step with MafA at a third timepoint. In another embodiment, the methods comprise contacting a mammalian non-pancreatic cell with PDX-1 and a second transcription factor at a first timepoint and contacting the cells from the first step with MafA at a second timepoint, wherein a second transcription factor is selected from NeuroD1, Ngn3, Sox9, and Pax4.

In another embodiment, the methods comprise contacting a mammalian non-pancreatic cell with PDX-1 and NeuroD1 at a first timepoint, and contacting the cells from the first step with MafA at a second timepoint. In another embodiment, the methods comprise contacting a mammalian non-pancreatic cell with PDX-1 and Pax4 at a first timepoint, and contacting the cells from the first step with MafA at a second timepoint. In another embodiment, the methods comprise contacting a mammalian non-pancreatic cell with PDX-1 and Ngn3 at a first timepoint, and contacting the cells from the first step with MafA at a second timepoint. In another embodiment, the methods comprise contacting a mammalian non-pancreatic cell with PDX-1 and Sox9 at a first timepoint, and contacting the cells from the first step with MafA at a second timepoint.

In another embodiment, the cells are contacted with all three factors (PDX-1; NeuroD1 or Pax4 or Ngn3; and MafA) at the same time but their expression levels are controlled in such a way as to have them expressed within the cell at a first timepoint for PDX-1, a second timepoint for NeuroD1 or Pax4 or Ngn3; and a third timepoint for MafA. The control of expression can be achieved by using appropriate promoters on each gene such that the genes are expressed sequentially, by modifying levels of mRNA, or by other means known in the art.

In some embodiments, the methods described herein further comprise contacting the cells at, before, or after any of the above steps with the transcription factor Sox-9.

In some embodiments, the first and second timepoints are identical resulting in contacting a cell population with two pTFs at a first timepoint, wherein at least one pTF comprises PDX-1, followed by contacting the resultant cell population with a third pTF at a second timepoint, wherein said third pTF is MafA.

The cell population that is exposed to, i.e., contacted with, the compounds (i.e. PDX-1, Pax-4, MafA, NeuroD1 and/or Sox-9 polypeptides or nucleic acid encoding PDX-1, Pax-4, MafA, NeuroD1 and/or Sox-9 polypeptides) can be any number of cells, i.e., one or more cells, and can be provided in vitro, in vivo, or ex vivo. The cell population that is contacted with the transcription factors can be expanded in vitro prior to being contacted with the transcription factors. The cell population produced exhibits a mature pancreatic beta cell phenotype. These cells can be expanded in vitro by methods known in the art prior to transdifferentiation and maturation along the β-cell lineage, and prior to administration or delivery to a patient or subject in need thereof.

In some embodiments, the second timepoint is at least 24 hours after the first timepoint. In an alternative embodiment, the second timepoint is less than 24 hours after the first timepoint. In another embodiment, the second timepoint is about 1 hour after the first timepoint, about 2 hours, about 3 hours, about 4 hours, about 5 hours, about 6 hours, about 7 hours, about 8 hours, about 9 hours, about 10 hours, about 11 hours, or about 12 hours after the first timepoint. In some embodiments, the second timepoint can be at least 24 hours, at least 48 hours, at least 72 hours, and at least 1 week or more after the first timepoint.

In another embodiment, the third timepoint is at least 24 hours after the second timepoint. In an alternative embodiment, the third timepoint is less than 24 hours after the second timepoint. In another embodiment, the third timepoint is at the same time as the second timepoint. In another embodiment, the third timepoint is about 1 hour after the second timepoint, about 2 hours, about 3 hours, about 4 hours, about 5 hours, about 6 hours, about 7 hours, about 8 hours, about 9 hours, about 10 hours, about 11 hours, or about 12 hours after the second timepoint. In other embodiments, the third timepoint can be at least 24 hours, at least 48 hours, at least 72 hours, and at least 1 week or more after the second timepoint.

In some embodiments, the first, second, and third timepoints are concurrent resulting in contacting a cell population with three pTFs at a single timepoint, wherein at least one pTF comprises PDX-1, at least one pTF comprises NeuroD1 or Pax4, and at least one pTF comprises MafA. In another embodiment, the first, second, and third timepoints are concurrent resulting in contacting a cell population with three pTFs at a single timepoint, wherein one pTF consists of PDX-1, one pTF consists of NeuroD1 or Pax4, and one pTF consists of MafA. A skilled artisan would appreciate that the term “timepoint” comprises a point in time, or a specific instant. In some embodiments, a timepoint comprises a short lapse of time. In some embodiments, a timepoint comprises less than 24 hours. In some embodiments, a timepoint comprises less than 12 hours. In some embodiments, a timepoint comprises less than 6 hours. In some embodiments, a timepoint comprises less than 3 hours. In some embodiments, a timepoint comprises less than 1 hour. In some embodiments, a timepoint comprises less than 30 minutes. In some embodiments, a timepoint comprises less than 10 minutes. In some embodiments, a timepoint comprises less than 5 minutes. In some embodiments, a timepoint comprises less than 1 minute. In some embodiments, a timepoint comprises less than 10 seconds.

In some embodiments, transcription factors comprise polypeptides, or ribonucleic acids or nucleic acids encoding the transcription factor polypeptides. In another embodiment, the transcription factor comprises a polypeptide. In another embodiment, the transcription factor comprises a nucleic acid sequence encoding the transcription factor. In another embodiment, the transcription factor comprises a Deoxyribonucleic acid sequence (DNA) encoding the transcription factor. In still another embodiment, the DNA comprises a cDNA. In another embodiment, the transcription factor comprises a ribonucleic acid sequence (RNA) encoding the transcription factor. In yet another embodiment, the RNA comprises an mRNA.

Transcription factors for use in the disclosure presented herein can be a polypeptide, ribonucleic acid or a nucleic acid. A skilled artisan would appreciate that the term “nucleic acid” may encompass DNA molecules (e.g., cDNA or genomic DNA), RNA molecules (e.g., mRNA, microRNA or other RNA derivatives), analogs of the DNA or RNA generated using nucleotide analogs, and derivatives, fragments and homologs thereof. The nucleic acid molecule can be single-stranded or double-stranded. In some embodiments, the nucleic acid is a DNA. In other embodiments, the nucleic acid is mRNA. mRNA is particularly advantageous in the methods disclosed herein, as transient expression of PDX-1 is sufficient to produce pancreatic beta cells. The polypeptide, ribonucleic acid or nucleic acid maybe delivered to the cell by means known in the art including, but not limited to, infection with viral vectors, electroporation and lipofection.

In some embodiments, the polypeptide, ribonucleic acid or nucleic acid is delivered to the cell by a viral vector. In some embodiments, the ribonucleic acid or nucleic acid is incorporated in an expression vector or a viral vector. In some embodiments, the viral vector is an adenovirus vector. In another embodiment, an adenoviral vector is a first generation adenoviral (FGAD) vector. In another embodiment, an FGAD is unable in integrate into the genome of a cell. In another embodiment, a FGAD comprises an E1-deleted recombinant adenoviral vector. In another embodiment, a FGAD provide transient expression of encoded polypeptides.

The expression or viral vector can be introduced to the cell by any of the following: transfection, electroporation, infection, or transduction. In other embodiments, the nucleic acid is mRNA and it is delivered for example by electroporation. In some embodiments, methods of electroporation comprise flow electroporation technology. For example, in another embodiment, methods of electroporation comprise use of a MaxCyte electroporation system (MaxCyte Inc. Georgia USA).

In certain embodiments, transcription factors for use in the methods described herein are selected from the group consisting of PDX-1, Pax-4, NeuroD1, and MafA. In other embodiments, transcription factors for use in the methods described herein are selected from the group consisting of PDX-1, Pax-4, NeuroD1, MafA, Ngn3, and Sox9.

The homeodomain protein PDX-1 (pancreatic and duodenal homeobox gene-1), also known as IDX-1, IPF-1, STF-1, or IUF-1, plays a central role in regulating pancreatic islet development and function. PDX-1 is either directly or indirectly involved in islet-cell-specific expression of various genes such as, for example insulin, glucagon, somatostatin, proinsulin convertase 1/3 (PC1/3), GLUT-2 and glucokinase. Additionally, PDX-1 mediates insulin gene transcription in response to glucose. Suitable sources of nucleic acids encoding PDX-1 include for example the human PDX-1 nucleic acid (and the encoded protein sequences) available as GenBank Accession Nos. U35632 and AAA88820, respectively. In some embodiments, the amino acid sequence of a PDX-1 polypeptide is set forth in SEQ ID NO: 3:

(SEQ ID NO: 3)
MNGEEQYYAATQLYKDPCAFQRGPAPEFSASPPACLYMGRQPPPPPP
HPFPGALGALEQGSPPDISPYEVPPLADDPAVAHLHHHLPAQLALPH
IPPAGPFPEGAEPGVLEEPNRVQLPFPWMKSTKAHAWKGQWAGGAYA
AEPEENKRTRTAYTRAQLLELEKEFLFNKYISRPRRVELAVMLNLTE
RHIKIWFQNRRMKWKKEEDKKRGGGTAVGGGGVAEPEQDCAVTSGEE
LLALPPPPPPGGAVPPAAPVAAREGRLPPGLSASPQPSSVAPRRPQE
PR.

In some embodiments, the nucleic acid sequence of a PDX-1 polynucleotide is set forth in SEQ ID NO: 4:

(SEQ ID NO: 4)
ATGAACGGCGAGGAGCAGTACTACGCGGCCACGCAGCTTTACAAGGA
CCCATGCGCGTTCCAGCGAGGCCCGGCGCCGGAGTTCAGCGCCAGCC
CCCCTGCGTGCCTGTACATGGGCCGCCAGCCCCCGCCGCCGCCGCCG
CACCCGTTCCCTGGCGCCCTGGGCGCGCTGGAGCAGGGCAGCCCCCC
GGACATCTCCCCGTACGAGGTGCCCCCCCTCGCCGACGACCCCGCGG
TGGCGCACCTTCACCACCACCTCCCGGCTCAGCTCGCGCTCCCCCAC
CCGCCCGCCGGGCCCTTCCCGGAGGGAGCCGAGCCGGGCGTCCTGGA
GGAGCCCAACCGCGTCCAGCTGCCTTTCCCATGGATGAAGTCTACCA
AAGCTCACGCGTGGAAAGGCCAGTGGGCAGGCGGCGCCTACGCTGCG
GAGCCGGAGGAGAACAAGCGGACGCGCACGGCCTACACGCGCGCACA
GCTGCTAGAGCTGGAGAAGGAGTTCCTATTCAACAAGTACATCTCAC
GGCCGCGCCGGGTGGAGCTGGCTGTCATGTTGAACTTGACCGAGAGA
CACATCAAGATCTGGTTCCAAAACCGCCGCATGAAGTGGAAAAAGGA
GGAGGACAAGAAGCGCGGCGGCGGGACAGCTGTCGGGGGTGGCGGGG
TCGCGGAGCCTGAGCAGGACTGCGCCGTGACCTCCGGCGAGGAGCTT
CTGGCGCTGCCGCCGCCGCCGCCCCCCGGAGGTGCTGTGCCGCCCGC
TGCCCCCGTTGCCGCCCGAGAGGGCCGCCTGCCGCCTGGCCTTAGCG
CGTCGCCACAGCCCTCCAGCGTCGCGCCTCGGCGGCCGCAGGAACCA
CGATGA.

Other sources of sequences for PDX-1 include rat PDX nucleic acid and protein sequences as shown in GenBank Accession No. U35632 and AAA18355, respectively, and are incorporated herein by reference in their entirety. An additional source includes zebrafish PDX-1 nucleic acid and protein sequences are shown in GenBank Accession No. AF036325 and AAC41260, respectively, and are incorporated herein by reference in their entirety.

Pax-4, also known as paired box 4, paired box protein 4, paired box gene 4, MODY9 and KPD, is a pancreatic-specific transcription factor that binds to elements within the glucagon, insulin and somatostatin promoters, and is thought to play an important role in the differentiation and development of pancreatic islet beta cells. In some cellular contexts, Pax-4 exhibits repressor activity. Suitable sources of nucleic acids encoding Pax-4 include for example the human Pax-4 nucleic acid (and the encoded protein sequences) available as GenBank Accession Nos. NM_006193.2 and AAD02289.1, respectively.

MafA, also known as V-maf musculoaponeurotic fibrosarcoma oncogene homolog A or RIPE3B1, is a beta-cell-specific and glucose-regulated transcriptional activator for insulin gene expression. MafA may be involved in the function and development of β cells as well as in the pathogenesis of diabetes. Suitable sources of nucleic acids encoding MafA include for example the human MafA nucleic acid (and the encoded protein sequences) available as GenBank Accession Nos. NM_201589.3 and NP_963883.2, respectively. In some embodiments, the amino acid sequence of a MafA polypeptide is set forth in SEQ ID NO: 5:

(SEQ ID NO: 5)
MAAELAMGAELPSSPLAIEYVNDFDLMKFEVKKEPPEAERFCHRLPP
GSLSSTPLSTPCSSVPSSPSFCAPSPGTGGGGGAGGGGGSSQAGGAP
GPPSGGPGAVGGTSGKPALEDLYWMSGYQHHLNPEALNLTPEDAVEA
LIGSGHHGAHHGAHHPAAAAAYEAFRGPGFAGGGGADDMGAGHHHGA
HHAAHHHHAAHHHHHHHHHHGGAGHGGGAGHHVRLEERFSDDQLVSM
SVRELNRQLRGFSKEEVIRLKQKRRTLKNRGYAQSCRFKRVQQRHIL
ESEKCQLQSQVEQLKLEVGRLAKERDLYKEKYEKLAGRGGPGSAGGA
GFPREPSPPQAGPCiGAKGTADFFL.

In another embodiment, the nucleic acid sequence of a MafA polynucleotide is set forth in SEQ ID NO: 6:

(SEQ ID NO: 6)
ATGGCCGCGGAGCTGGCGATGGGCGCCGAGCTGCCCAGCAGCCCGC
TGGCCATCGAGTACGTCAACGACTTCGACCTGATGAAGTTCGAGGT
GAAGAAGGAGCCTCCCGAGGCCGAGCGCTTCTGCCACCGCCTGCCG
CCAGGCTCGCTGTCCTCGACGCCGCTCAGCACGCCCTGCTCCTCCG
TGCCCTCCTCGCCCAGCTTCTGCGCGCCCAGCCCGGGCACCGGCGG
CGGCGGCGGCGCGGGGGGCGGCGGCGGCTCGTCTCAGGCCGGGGGC
GCCCCCGGGCCGCCGAGCGGGGGCCCCGGCGCCGTCGGGGGCACCT
CGGGGAAGCCGGCGCTGGAGGATCTGTACTGGATGAGCGGCTACCA
GCATCACCTCAACCCCGAGGCGCTCAACCTGACGCCCGAGGACGCG
GTGGAGGCGCTCATCGGCAGCGGCCACCACGGCGCGCACCACGGCG
CGCACCACCCGGCGGCCGCCGCAGCCTACGAGGCTTTCCGCGGCCC
GGGCTTCGCGGGCGGCGGCGGAGCGGACGACATGGGCGCCGGCCAC
CACCACGGCGCGCACCACGCCGCCCACCACCACCACGCCGCCCACC
ACCACCACCACCACCACCACCATGGCGGCGCGGGACACGGCGGTGG
CGCGGGCCACCACGTGCGCCTGGAGGAGCGCTTCTCCGACGACCAG
CTGGTGTCCATGTCGGTGCGCGAGCTGAACCGGCAGCTCCGCGGCT
TCAGCAAGGAGGAGGTCATCCGGCTCAAGCAGAAGCGGCGCACGCT
CAAGAACCGCGGCTACGCGCAGTCCTGCCGCTTCAAGCGGGTGCAG
CAGCGGCACATTCTGGAGAGCGAGAAGTGCCAACTCCAGAGCCAGG
TGGAGCAGCTGAAGCTGGAGGTGGGGCGCCTGGCCAAAGAGCGGGA
CCTGTACAAGGAGAAATACGAGAAGCTGGCGGGCCGGGGCGGCCCC
GGGAGCGCGGGCGGGGCCGGTTTCCCGCGGGAGCCTTCGCCGCCGC
AGGCCGGTCCCGGCGGGGCCAAGGGCACGGCCGACTTCTTCCTGTA
G

Neurog3, also known as neurogenin 3 or Ngn3, is a basic helix-loop-helix (bHLH) transcription factor required for endocrine development in the pancreas and intestine. Suitable sources of nucleic acids encoding Neurog3 include for example the human Neurog3 nucleic acid (and the encoded protein sequences) available as GenBank Accession Nos. NM_020999.3 and NP_066279.2, respectively.

NeuroD1, also known as Neuro Differentiation 1 or NeuroD, and beta-2 (β2) is a Neuro D-type transcription factor. It is a basic helix-loop-helix transcription factor that forms heterodimers with other bHLH proteins and activates transcription of genes that contain a specific DNA sequence known as the E-box. It regulates expression of the insulin gene, and mutations in this gene result in type II diabetes mellitus. Suitable sources of nucleic acids encoding NeuroD1 include for example the human NeuroD1 nucleic acid (and the encoded protein sequences) available as GenBank Accession Nos. NM_002500.4 and NP_002491.2, respectively.

In some embodiments, the amino acid sequence of a NeuroD1 polypeptide is set forth in SEQ ID NO: 7:

(SEQ ID NO: 7)
MTKSYSESGLMGEPQPQGPPSWTDECLSSQDEEHEADKKEDDLETM
NAEEDSLRNGGEEEDEDEDLEEEEEEEEEDDDQKPKRRGPKKKKMT
KARLEREKLRRMKANARERNRMHGLNAALDNLRKVVPCYSKTQKLS
KIETLRLAKNYIWALSEILRSGKSPDLVSEVQTLCKGLSQPTTNLV
AGCLQLNPRTFLPEQNQDMPPHLPTASASEPVHPYSYQSPGLPSPP
YGTMDSSHVFHVKPPPHAYSAALEPFFESPLTDCTSPSFDGPLSPP
LSINGNFSFKHEPSAEFEKNYAFTMHYPAATLAGAQSHGSIFSGTA
APRCEIPIDNIMSFDSHSHHERVMSAQLNAIFHD.

In another embodiment, the nucleic acid sequence of a NeuroD1 polynucleotide is set forth in SEQ ID NO: 8:

(SEQ ID NO: 8)
ATGACCAAATCGTACAGCGAGAGTGGGCTGATGGGCGAGCCTCAGC
CCCAAGGTCCTCCAAGCTGGACAGACGAGTGTCTCAGTTCTCAGGA
CGAGGAGCACGAGGCAGACAAGAAGGAGGACGACCTCGAAGCCATG
AACGCAGAGGAGGACTCACTGAGGAACGGGGGAGAGGAGGAGGACG
AAGATGAGGACCTGGAAGAGGAGGAAGAAGAGGAAGAGGAGGATGA
CGATCAAAAGCCCAAGAGACGCGGCCCCAAAAAGAAGAAGATGACT
AAGGCTCGCCTGGAGCGTTTTAAATTGAGACGCATGAAGGCTAACG
CCCGGGAGCGGAACCGCATGCACGGACTGAACGCGGCGCTAGACAA
CCTGCGCAAGGTGGTGCCTTGCTATTCTAAGACGCAGAAGCTGTCC
AAAATCGAGACTCTGCGCTTGGCCAAGAACTACATCTGGGCTCTGT
CGGAGATCTCGCGCTCAGGCAAAAGCCCAGACCTGGTCTCCTTCGT
TCAGACGCTTTGCAAGGGCTTATCCCAACCCACCACCAACCTGGTT
GCGGGCTGCCTGCAACTCAATCCTCGGACTTTTCTGCCTGAGCAGA
ACCAGGACATGCCCCCGCACCTGCCGACGGCCAGCGCTTCCTTCCC
TGTACACCCCTACTCCTACCAGTCGCCTGGGCTGCCCAGTCCGCCT
TACGGTACCATGGACAGCTCCCATGTCTTCCACGTTAAGCCTCCGC
CGCACGCCTACAGCGCAGCGCTGGAGCCCTTCTTTGAAAGCCCTCT
GACTGATTGCACCAGCCCTTCCTTTGATGGACCCCTCAGCCCGCCG
CTCAGCATCAATGGCAACTTCTCTTTCAAACACGAACCGTCCGCCG
AGTTTGAGAAAAATTATGCCTTTACCATGCACTATCCTGCAGCGAC
ACTGGCAGGGGCCCAAAGCCACGGATCAATCTTCTCAGGCACCGCT
GCCCCTCGCTGCGAGATCCCCATAGACAATATTATGTCCTTCGATA
GCCATTCACATCATGAGCGAGTCATGAGTGCCCAGCTCAATGCCAT
ATTTCATGATTAG.

Sox9 is a transcription factor that is involved in embryonic development. Sox9 has been particularly investigated for its importance in bone and skeletal development. SOX-9 recognizes the sequence CCTTGAG along with other members of the HMG-box class DNA-binding proteins. In the context of the disclosure presented herein, the use of Sox9 may be involved in maintaining the pancreatic progenitor cell mass, either before or after induction of transdifferentiation. Suitable sources of nucleic acids encoding Sox9 include for example the human Sox9 nucleic acid (and the encoded protein sequences) available as GenBank Accession Nos. NM_000346.3 and NP_000337.1, respectively.

Homology is, in some embodiments, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID No: 1-8 of greater than 60%. In another embodiment, “homology” refers to identity to a sequence selected from SEQ ID No: 1-8 of greater than 70%. In another embodiment, the identity is greater than 75%, greater than 78%, greater than 80%, greater than 82%, greater than 83%, greater than 85%, greater than 87%, greater than 88%, greater than 90%, greater than 92%, greater than 93%, greater than 95%, greater than 96%, greater than 97%, greater than 98%, or greater than 99%. In another embodiment, the identity is 100%. Each possibility represents a separate embodiment of the disclosure presented herein.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example, methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

Protein and/or peptide homology for any amino acid sequence listed herein is determined, in some embodiments, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the disclosure presented herein.

Another embodiment disclosed herein, pertains to vectors. In some embodiments, a vector used in the methods disclosed herein comprises an expression vector. In another embodiment, an expression vector comprises a nucleic acid encoding a PDX-1, Pax-4, NeuroD1 or MafA protein, or other pancreatic transcription factor, such as Ngn3, or derivatives, fragments, analogs, homologs or combinations thereof. In some embodiments, the expression vector comprises a single nucleic acid encoding any of the following transcription factors: PDX-1, Pax-4, NeuroD1, Ngn3, MafA, or Sox-9 or derivatives or fragments thereof. In some embodiments, the expression vector comprises two nucleic acids encoding any combination of the following transcription factors: PDX-1, Pax-4, NeuroD1, Ngn3, MafA, or Sox-9 or derivatives or fragments thereof. In a yet another embodiment, the expression vector comprises nucleic acids encoding PDX-1 and NeuroD1. In a still another embodiment, the expression vector comprises nucleic acids encoding PDX-1 and Pax4. In another embodiment, the expression vector comprises nucleic acids encoding MafA.

A skilled artisan would appreciate that the term “vector” encompasses a nucleic acid molecule capable of transporting another nucleic acid to which it has been linked. One type of vector is a “plasmid”, which encompasses a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. A skilled artisan would appreciate that the terms “plasmid” and “vector” may be used interchangeably having all the same qualities and meanings. In some embodiments, the term “plasmid” is the most commonly used form of vector. However, the disclosure presented herein is intended to include such other forms of expression vectors, such as viral vectors (e.g., replication defective retroviruses, lentivirus, adenoviruses and adeno-associated viruses), which serve equivalent functions. Additionally, some viral vectors are capable of targeting a particular cell type either specifically or non-specifically.

The recombinant expression vectors disclosed herein comprise a nucleic acid disclosed herein, in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, a skilled artisan would appreciate that the term “operably linked” may encompass nucleotide sequences of interest linked to the regulatory sequence(s) in a manner that allows for expression of the nucleotide sequence (e.g., in an in vitro transcription/translation system or in a host cell when the vector is introduced into the host cell). A skilled artisan would appreciate that term “regulatory sequence” may encompass promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors disclosed here may be introduced into host cells to thereby produce proteins or peptides, including fusion proteins or peptides, encoded by nucleic acids as described herein (e.g., PDX-1, Pax-4, MafA, NeuroD1 or Sox-9 proteins, or mutant forms or fusion proteins thereof, etc.).

For example, an expression vector comprises one nucleic acid encoding a transcription factor operably linked to a promoter. In expression vectors comprising two nucleic acids encoding transcription factors, each nucleic acid may be operably linked to a promoter. The promoter operably linked to each nucleic acid may be different or the same. Alternatively, the two nucleic acids may be operably linked to a single promoter. Promoters useful for the expression vectors disclosed here could be any promoter known in the art. The ordinarily skilled artisan could readily determine suitable promoters for the host cell in which the nucleic acid is to be expressed, the level of expression of protein desired, or the timing of expression, etc. The promoter may be a constitutive promoter, an inducible promoter, or a cell-type specific promoter.

The recombinant expression vectors disclosed here can be designed for expression of PDX-1 in prokaryotic or eukaryotic cells. For example, PDX-1, Pax-4, MafA, NeuroD1, and/or Sox-9 can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

In another embodiment, the PDX-1, Pax-4, MafA, NeuroD1, or Sox-9 expression vector is a yeast expression vector. Examples of vectors for expression in yeast S. cerevisiae include pYepSec1 (Baldari, et al., (1987) EMBO J 6:229-234), pMFa (Kujan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, PDX-1, Pax-4, MafA, NeuroD1 or Sox-9 can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid disclosed here is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally regulated promoters are also encompassed, e.g., the murine hox promoters (Kessel and Gruss (1990) Science 249:374-379) and the alpha-fetoprotein promoter (Campes and Tilghman (1989) Genes Dev 3:537-546).

Another embodiment disclosed herein pertains to host cells into which a recombinant expression vector disclosed here has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. Additionally, host cells could be modulated once expressing PDX-1, Pax-4, MafA, NeuroD1 or Sox-9 or a combination thereof, and may either maintain or loose original characteristics.

Vector DNA may be introduced into cells via conventional transformation, transduction, infection or transfection techniques. A skilled artisan would appreciate that the terms “transformation” “transduction”, “infection” and “transfection” may encompass a variety of art-recognized techniques for introducing foreign nucleic acid (e.g., DNA) into a host cell, including calcium phosphate or calcium chloride co-precipitation, DEAE-dextran-mediated transfection, lipofection, or electroporation. In addition, transfection can be mediated by a transfection agent. A skilled artisan would appreciate that the term “transfection agent” may encompass any compound that mediates incorporation of DNA in the host cell, e.g., liposome. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

Transfection may be “stable” (i.e. integration of the foreign DNA into the host genome) or “transient” (i.e., DNA is episomally expressed in the host cells) or mRNA is electroporated into cells).

For stable transfection of mammalian cells, it is known that, depending upon the expression vector and transfection technique used, only a small fraction of cells may integrate the foreign DNA into their genome the remainder of the DNA remains episomal. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding PDX-1 or can be introduced on a separate vector. Cells stably transfected with the introduced nucleic acid can be identified by drug selection (e.g., cells that have incorporated the selectable marker gene will survive, while the other cells die). In another embodiment, the cells modulated by PDX-1 or the transfected cells are identified by the induction of expression of an endogenous reporter gene. In some embodiments, the promoter is the insulin promoter driving the expression of green fluorescent protein (GFP).

In some embodiments the PDX-1, Pax-4, MafA, NeuroD1, or Sox-9 nucleic acid is present in a viral vector. In some embodiments, the PDX-1 and NeuroD1 nucleic acids are present in the same viral vector. In another embodiment, the PDX-1 and Pax4 nucleic acids are present in the same viral vector. In another embodiment the PDX-1, Pax-4, MafA, NeuroD1, or Sox-9 nucleic acid is encapsulated in a virus. In another embodiment, the PDX-1 and NeuroD1 is encapsulated in a virus (i.e., nucleic acids encoding PDX-1 and NeuroD1 are encapsulated in the same virus particle). In another embodiment, the PDX-1 and Pax4 are encapsulated in a virus (i.e., nucleic acids encoding PDX-1 and Pax4 are encapsulated in the same virus particle). In some embodiments, the virus infects pluripotent cells of various tissue types, e.g. hematopoietic stem, cells, neuronal stem cells, hepatic stem cells or embryonic stem cells. In some embodiments, the virus is hepatotropic. A skilled artisan would appreciate that the term “hepatotropic” it is meant that the virus has the capacity to target the cells of the liver either specifically or non-specifically. In further embodiments, the virus is a modulated hepatitis virus, SV-40, or Epstein-Bar virus. In yet another embodiment, the virus is an adenovirus.

In some embodiments, 3D cell clusters are dissociated into single cells. In some embodiments, dissociating can be effectuated with any enzyme or combination of enzymes having proteolytic and/or collagenolytic activity. In some embodiments, dissociation is effectuated with trypsin, collagenase, hyaluronidase, papain, protease type XIV, pronase and/or proteinase K. In some embodiments, dissociation is effectuated with Accutase®. In some embodiments, dissociated cells are further seeded in adherent conditions.

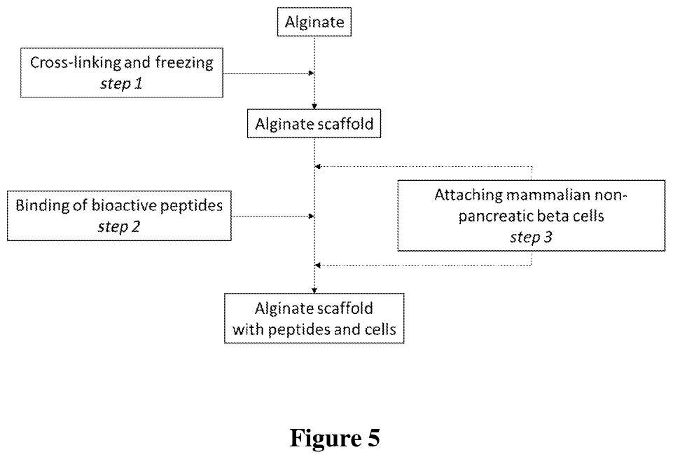

FIG. 4 describes one embodiment of a manufacturing process of human insulin producing cells, wherein the starting material comprises liver tissue. A skilled artisan would recognize that any source of non-pancreatic β-cell tissue could be used in this manufacturing process.

Embodiments for many of the steps presented in FIG. 4 are described in detail throughout this application, and will not be repeated herein, though they should be considered herein. Reference is also made to Examples 1-2, which exemplify many of these steps. In brief, the manufacturing process may be understood based on the steps presented below.

As indicated at Step 1: Obtaining Liver Tissue. In some embodiments, liver tissue is human liver tissue. In another embodiment, the liver tissue is obtained as part of a biopsy. In another embodiment, liver tissue is obtained by way of any surgical procedure known in the art. In another embodiment, obtaining liver tissue is performed by a skilled medical practitioner. In another embodiment, liver tissue obtained is liver tissue from a healthy individual. In a related embodiment, the healthy individual is an allogeneic donor for a patient in need of a cell-based therapy that provides processed insulin in a glucose regulated manner, for example a type I Diabetes mellitus patient or a patient suffering for pancreatitis. In another embodiment, donor Screening and Donor Testing was performed to ensure that tissue obtained from donors shows no clinical or physical evidence of or risk factors for infectious or malignant diseases were from manufacturing of AIP cells. In yet another embodiment, liver tissue is obtained from a patient in need of a cell-based therapy that provides processed insulin in a glucose regulated manner, for example a type I Diabetes mellitus patient or a patient suffering for pancreatitis. In still another embodiment, liver tissue is autologous with a patient in need of a cell-based therapy that provides processed insulin in a glucose regulated manner, for example a type I Diabetes mellitus patient or a patient suffering for pancreatitis.

As indicated at Step 2: Recovery and Processing of Primary Liver Cells. Liver tissue is processed using well know techniques in the art for recovery of adherent cells to be used in further processing. In some embodiments, liver tissue is cut into small pieces of about 1-2 mm and gently pipetted up and down in sterile buffer solution. The sample may then be incubated with collagenase to digest the tissue. Following a series of wash steps, in another embodiment, primary liver cells may be plated on pre-treated fibronectin-coated tissue culture tissue dishes. A skilled artisan would then process (passage) the cells following well-known techniques for propagation of liver cells. Briefly, cells may be grown in a propagation media and through a series of seeding and harvesting cell number is increased. Cells may be split upon reaching 80% confluence and re-plated. In another embodiment, following wash steps, primary liver cells are seeded under non-adherent conditions. In one embodiment, following wash steps, primary liver cells are attached to a scaffold.

A skilled artisan would appreciate the need for sufficient cells at, for example the 2-week time period, prior to beginning the expansion phase of the protocol (step 3). The skilled artisan would recognize that the 2-week time period is one example of a starting point for expanding cells, wherein cells may be ready for expansion be before or after this time period. In some embodiments, recovery and processing of primary cells yields at least 1×10⁵ cells after two passages of the cells. In another embodiment, recovery and processing of primary cells yields at least 1×10⁶ cells after two passages of the cells. In another embodiment, recovery and processing of primary cells yields at least 2×10⁶ cells after two passages of the cells. In another embodiment, recovery and processing of primary cells yields at least 5×10⁶ cells after two passages of the cells. In another embodiment, recovery and processing of primary cells yields at least 1×10⁷ cells after two passages of the cells. In another embodiment, recovery and processing of primary cells yields between 1×10⁵-1×10⁶ cells after two passages of the cells. In another embodiment, recovery and processing of primary cells yields between 1×10⁶-1×10⁷ cells after two passages of the cells. In another embodiment, enough starting tissue is used to ensure the recovery and processing of primary cells yields enough cells after two passages for an adequate seeding density at Step 3, large-scale expansion of the cells.

In another embodiment, early passage primary cells are cryopreserved for later use. In some embodiments, 1^{st} passage primary cells are cryopreserved for later use. In yet another embodiment, 2^{nd} passage primary cells are cryopreserved for later use.

As indicated at Step 3: Propagation/Proliferation of Primary Liver Cells. Step 3 represents the large-scale expansion phase of the manufacturing process. In some embodiments, cells propagate/proliferate on a scaffold. A skilled artisan would appreciate the need for sufficient cells at the 5-week time period, prior to beginning the transdifferentiation phase of the protocol (step 4), wherein a predetermined number of cells may be envisioned to be needed for treating a patient. In some embodiments, the predetermined number of cells needed prior to transdifferentiation comprises about 1×10⁸ primary cells. In another embodiment, the predetermined number of cells needed prior to transdifferentiation comprises about 2×10⁸ primary cells. In some embodiments, the predetermined number of cells needed prior to transdifferentiation comprises about 3×10⁸ primary cells, 4×10⁸ primary cells, 5×10⁸ primary cells, 6×10⁸ primary cells, 7×10⁸ primary cells, 8×10⁸ primary cells, 9×10⁸ primary cells, 1×10⁹ primary cells, 2×10⁹ primary cells, 3×10⁹ primary cells, 4×10⁹ primary cells, 5×10⁹ primary cells, 6×10⁹ primary cells, 7×10⁹ primary cells, 8×10⁹ primary cells, 9×10⁹ primary cells, or 1×10¹⁰ primary cells.

In some embodiments, cells are seeded on a scaffold. In some embodiments, the cell seeding density at the time of expansion comprises 1×10³-10×10³ cell/cm². In another embodiment, the cell seeding density at the time of expansion comprises 1×10³—8×10³ cell/cm². In another embodiment, the cell seeding density at the time of expansion comprises 1×10³-5×10³ cell/cm². In another embodiment, the cell seeding density at the time of expansion comprises 1×10³. In another embodiment, the cell seeding density at the time of expansion comprises 2×10³. In another embodiment, the cell seeding density at the time of expansion comprises 3×10³. In another embodiment, the cell seeding density at the time of expansion comprises 4×10³. In another embodiment, the cell seeding density at the time of expansion comprises 5×10³. In another embodiment, the cell seeding density at the time of expansion comprises 6×10³. In another embodiment, the cell seeding density at the time of expansion comprises 7×10³. In another embodiment, the cell seeding density at the time of expansion comprises 8×10³. In another embodiment, the cell seeding density at the time of expansion comprises 9×10³. In another embodiment, the cell seeding density at the time of expansion comprises 10×10³.

In another embodiment, the range for cells seeding viability at the time of expansion comprises 60-100%. In another embodiment, the range for cells seeding viability at the time of expansion comprises a viability of about 70-99%. In another embodiment, the cell seeding viability at the time of expansion comprises a viability of about 60%. In another embodiment, the cell seeding viability at the time of expansion comprises a viability of about 65%. In another embodiment, the cell seeding viability at the time of expansion comprises a viability of about 70%. In another embodiment, the cell seeding viability at the time of expansion comprises a viability of about 75%. In another embodiment, the cell seeding viability at the time of expansion comprises a viability of about 80%. In another embodiment, the cell seeding viability at the time of expansion comprises a viability of about 85%. In another embodiment, the cell seeding viability at the time of expansion comprises a viability of about 90%. In another embodiment, the cell seeding viability at the time of expansion comprises a viability of about 95%. In another embodiment, the cell seeding viability at the time of expansion comprises a viability of about 99%. In another embodiment, the cell seeding viability at the time of expansion comprises a viability of about 99.9%.

A skilled artisan would recognize variability within starting tissue material. Therefore, in another embodiment expansion occurs between weeks 2 and 6. In still another embodiment, expansion occurs between weeks 2 and 7. In another embodiment, expansion occurs between weeks 2 and 4. In yet another embodiment, expansion occurs until the needed number of primary cells has been propagated.

In some embodiments, bioreactors are used to expand and propagate primary cells prior to the transdifferentiation step. In some embodiments, cells aggregated in 3D clusters attached to a scaffold are propagated in bioreactors. Bioreactors may be used or cultivation of cells, in which conditions are suitable for high cell concentrations. In another embodiment, a bioreactor provides a closed system for expansion of cells. In another embodiment, multiple bioreactors are used in a series for cell expansion. In another embodiment, a bioreactor used in the methods disclosed herein is a single use bioreactor. In another embodiment, a bioreactor used is a multi-use bioreactor. In yet another embodiment, a bioreactor comprises a control unit for monitoring and controlling parameters of the process. In another embodiment, parameters for monitoring and controlling comprise Dissolve Oxygen (DO), pH, gases, and temperature.

In some embodiments, primary liver cells are propagated under non-adherent conditions. In some embodiments, primary liver cells are attached to a scaffold. In some embodiments, primary liver cells are propagated on a scaffold.

As indicated at Step 4: Transdifferentiation (TD) of primary Liver Cells. In some embodiments, transdifferentiation comprises any method of transdifferentiation disclosed herein. For example, transdifferentiation may comprise a “hierarchy” (1+1+1) protocol or a “2+1” protocol, as disclosed herein. In some embodiments, a “hierarchy” or 1+1+1 protocol refers to a protocol in which 3 pTFs are administered in a sequential manner and according to the order in which they're expressed during pancreatic beta cell differentiation. In some embodiment, the 3 pTFs are PDX-1, NeuroD1 and MafA. In some embodiments, “2+1” protocol refers to a transdifferentiation protocol in which 2 pTFs are administered at a first time and a third pTF is administered at a subsequent second time.

In some embodiments, the resultant cell population following transdifferentiation comprises transdifferentiated cells having a pancreatic phenotype and function. In another embodiment, the resultant cell population following transdifferentiation comprises transdifferentiated cells having a mature β-cell pancreatic phenotype and function. In another embodiment, the resultant cell population following transdifferentiation comprises transdifferentiated cells having increased insulin content. In another embodiment, the resultant cell population following transdifferentiation comprises transdifferentiated cells able to secrete processed insulin in a glucose-regulated manner. In another embodiment, the resultant cell population following transdifferentiation comprises transdifferentiated cells has increased C-peptide levels.

In another embodiment, the resultant cell population following transdifferentiation comprises transdifferentiated cells having increased endogenous expression of at least one pancreatic gene marker. In another embodiment, endogenous expression is increased for at least two pancreatic gene markers. In another embodiment, endogenous expression is increased for at least three pancreatic gene markers. In another embodiment, endogenous expression is increased for at least four pancreatic gene markers. In a related embodiment, pancreatic gene markers comprise PDX-1, NeuroD1, MafA, Nkx6.1, glucagon, somatostatin and Pax4.

In some embodiments, endogenous PDX-1 expression is greater than 10² fold over non-transdifferentiated cells. In another embodiment, endogenous PDX-1 expression is greater than 10³ fold over non-transdifferentiated cells. In another embodiment, endogenous PDX-1 expression is greater than 10⁴ fold over non-transdifferentiated cells. In another embodiment, endogenous PDX-1 expression is greater than 10⁵ fold over non-transdifferentiated cells. In another embodiment, endogenous PDX-1 expression is greater than 10⁶ fold over non-transdifferentiated cells.

In another embodiment, endogenous NeuroD1 expression is greater than 10² fold over non-transdifferentiated cells. In another embodiment, endogenous NeuroD1 expression is greater than 10³ fold over non-transdifferentiated cells. In another embodiment, endogenous NeuroD1 expression is greater than 10⁴ fold over non-transdifferentiated cells. In another embodiment, endogenous NeuroD1 expression is greater than 10⁵ fold over non-transdifferentiated cells.

In another embodiment, endogenous MafA expression is greater than 10² fold over non-transdifferentiated cells. In another embodiment, endogenous MafA expression is greater than 10³ fold over non-transdifferentiated cells. In another embodiment, endogenous MafA expression is greater than 10⁴ fold over non-transdifferentiated cells. In another embodiment, endogenous MafA expression is greater than 10⁵ fold over non-transdifferentiated cells.

In another embodiment, endogenous glucagon expression is greater than 10 fold over non-transdifferentiated cells. In another embodiment, endogenous glucagon expression is greater than 10² fold over non-transdifferentiated cells. In another embodiment, endogenous glucagon expression is greater than 10³ fold over non-transdifferentiated cells.

In another embodiment, endogenous expression of PDX-1, NeuroD1, or MafA, or any combination thereof is each greater than 60% over non-transdifferentiated cells. In another embodiment, endogenous expression of PDX-1, NeuroD1, or MafA, or any combination thereof is each greater than 70% over non-transdifferentiated cells. In another embodiment, endogenous expression of PDX-1, NeuroD1, or MafA, or any combination thereof is each greater than 80% over non-transdifferentiated cells

In another embodiment, the resultant cell population following transdifferentiation comprises transdifferentiated cells having at least 60% viability. In another embodiment, the resultant cell population following transdifferentiation comprises transdifferentiated cells having at least 70% viability. In another embodiment, the resultant cell population following transdifferentiation comprises transdifferentiated cells having at least 80% viability. In another embodiment, the resultant cell population following transdifferentiation comprises transdifferentiated cells having at least 90% viability.

In some embodiments, the cells exhibiting a mature beta-cell phenotype generated by the methods described herein may repress at least one gene or the gene expression profile of the original cell. For example, a liver cell that is induced to exhibit a mature beta-cell phenotype may repress at least one liver-specific gene. One skilled in the art could readily determine the liver-specific gene expression of the original cell and the produced cells using methods known in the art, i.e. measuring the levels of mRNA or polypeptides encoded by the genes. Upon comparison, a decrease in the liver-specific gene expression would indicate that transdifferentiation has occurred.

In certain embodiments, the transdifferentiated cells disclosed herein comprise a reduction of liver phenotypic markers. In some embodiments, there is a reduction of expression of albumin, alpha-1 anti-trypsin, or a combination thereof. In another embodiment, less than 5% of the cell population expressing endogenous PDX-1 expresses albumin and alpha-1 anti-trypsin. In another embodiment, less than 10%, 9%, 8%, 7%, 6%, 4%, 3%, 2%, or 1% of the transdifferentiated cells expressing endogenous PDX-1 expresses albumin and alpha-1 anti-trypsin.

In another embodiment, transdifferentiated cells maintain a pancreatic phenotype and function for at least 6 months. In another embodiment, transdifferentiated cells maintain a pancreatic phenotype and function for at least 12 months. In another embodiment, transdifferentiated cells maintain a pancreatic phenotype and function for at least 18 months. In another embodiment, transdifferentiated cells maintain a pancreatic phenotype and function for at least 24 months. In another embodiment, transdifferentiated cells maintain a pancreatic phenotype and function for at least 36 months. In another embodiment, transdifferentiated cells maintain a pancreatic phenotype and function for at least 48 months. In another embodiment, transdifferentiated cells maintain a pancreatic phenotype and function for at least 4 years. In another embodiment, transdifferentiated cells maintain a pancreatic phenotype and function for at least 5 years.

In some embodiments, cell number is maintained during transdifferentiation. In another embodiment, cell number decreases by less than 5% during transdifferentiation. In another embodiment, cell number decreases by less than 10% during transdifferentiation. In another embodiment, cell number decreases by less than 15% during transdifferentiation. In another embodiment, cell number decreases by less than 20% during transdifferentiation. In another embodiment, cell number decreases by less than 25% during transdifferentiation.

In some embodiments, primary liver cells are transdifferentiated under non-adherent conditions. In some embodiments, primary liver cells are seeded on a scaffold and transdifferentiated on it.

In some embodiments, the cell seeding density comprises 1×10³-10×10³ cell/cm². In another embodiment, the cell seeding density comprises 1×10³-8×10³ cell/cm². In another embodiment, the cell seeding density comprises 1×10³-5×10³ cell/cm². In another embodiment, the cell seeding density comprises 1×10³. In another embodiment, the cell seeding density comprises 2×10³. In another embodiment, the cell seeding density comprises 3×10³. In another embodiment, the cell seeding density comprises 4×10³. In another embodiment, the cell seeding density comprises 5×10³. In another embodiment, the cell seeding density comprises 6×10³. In another embodiment, the cell seeding density comprises 7×10³. In another embodiment, the cell seeding density comprises 8×10³. In another embodiment, the cell seeding density comprises 9×10³. In another embodiment, the cell seeding density comprises 10×10³.

In some embodiments, the seeded cells are in contact with a medium. In some embodiments, cells are seeded at a density of 5×10³ to 10×10³ cells/ml. In some embodiments, cells are seeded at a density of 10×10³ to 20×10³ cells/ml. In some embodiments, cells are seeded at a density of 20×10³ to 30×10³ cells/ml. In some embodiments, cells are seeded at a density of 30×10³ to 40×10³ cells/ml. In some embodiments, cells are seeded at a density of 40×10³ to 50×10³ cells/ml. In some embodiments, cells are seeded at a density of 50×10³ to 60×10³ cells/ml. In some embodiments, cells are seeded at a density of 60×10³ to 70×10³ cells/ml. In some embodiments, cells are seeded at a density of 70×10³ to 80×10³ cells/ml. In some embodiments, cells are seeded at a density of 80×10³ to 90×10³ cells/ml. In some embodiments, cells are seeded at a density of 90×10³ to 100×10³ cells/ml. In some embodiments, cells are seeded at a density of 100×10³ to 200×10³ cells/ml. In some embodiments, cells are seeded at a density of 200×10³ to 500×10³ cells/ml. In some embodiments, cells are seeded at a density of over 500×10³ cells/ml.

In some embodiments, the density of transdifferentiated cells on the scaffold at the end of the production process is about 1×10³-1×10⁵ cells/cm². In another embodiment, the density of transdifferentiated cells on the scaffold at the end of the production process is about 1×10⁴-5×10⁴ cells/cm². In another embodiment, the density of transdifferentiated cells on the scaffold at the end of the production process is about 1×10⁴-4×10⁴ cells/cm². In another embodiment, the density of transdifferentiated cells on the scaffold at the end of the production process is about 1×10³ cells/cm². In another embodiment, the density of transdifferentiated cells on the scaffold at the end of the production process is about 2×10³ cells/cm². In another embodiment, the density of transdifferentiated cells on the scaffold at the end of the production process is about 3×10³ cells/cm². In another embodiment, the density of transdifferentiated cells on the scaffold at the end of the production process is about 4×10³ cells/cm². In another embodiment, the density of transdifferentiated cells on the scaffold at the end of the production process is about 5×10³ cells/cm². In another embodiment, the density of transdifferentiated cells on the scaffold at the end of the production process is about 6×10³ cells/cm². In another embodiment, the density of transdifferentiated cells on the scaffold at the end of the production process is about 7×10³ cells/cm². In another embodiment, the density of transdifferentiated cells on the scaffold at the end of the production process is about 8×10³ cells/cm². In another embodiment, the density of transdifferentiated cells on the scaffold at the end of the production process is about 9×10³ cells/cm². In another embodiment, the density of transdifferentiated cells on the scaffold at the end of the production process is about 1×10⁴ cells/cm². In another embodiment, the density of transdifferentiated cells on the scaffold at the end of the production process is about 2×10⁴ cells/cm². In another embodiment, the density of transdifferentiated cells on the scaffold at the end of the production process is about 3×10⁴ cells/cm². In another embodiment, the density of transdifferentiated cells on the scaffold at the end of the production process is about 4×10⁴ cells/cm². In another embodiment, the density of transdifferentiated cells on the scaffold at the end of the production process is about 5×10⁴ cells/cm². In another embodiment, the density of transdifferentiated cells on the scaffold at the end of the production process is about 6×10⁴ cells/cm². In another embodiment, the density of transdifferentiated cells on the scaffold at the end of the production process is about 7×10⁴ cells/cm². In another embodiment, the density of transdifferentiated cells on the scaffold at the end of the production process is about 8×10⁴ cells/cm². In another embodiment, the density of transdifferentiated cells on the scaffold at the end of the production process is about 9×10⁴ cells/cm².

In another embodiment, the range for cell viability at the end of the production process comprises 50-100%. In another embodiment, the range for cell viability at the end of the production process comprises 60-100%. In another embodiment, the range for cell viability at the end of the production process comprises 50-90%. In another embodiment, the range for cell viability at the end of the production process comprises a viability of about 60-99%. In another embodiment, the range for cell viability at the end of the production process comprises a viability of about 60-90%. In another embodiment, the cell viability at the end of the production process comprises a viability of about 60%. In another embodiment, the cell viability at the end of the production process comprises a viability of about 65%. In another embodiment, the cell viability at the end of the production process comprises a viability of about 70%. In another embodiment, the cell viability at the end of the production process comprises a viability of about 75%. In another embodiment, the cell viability at the end of the production process comprises a viability of about 80%. In another embodiment, the cell viability at the end of the production process comprises a viability of about 85%. In another embodiment, the cell viability at the end of the production process comprises a viability of about 90%. In another embodiment, the cell viability at the end of the production process comprises a viability of about 95%. In another embodiment, the cell viability at the end of the production process comprises a viability of about 99%. In another embodiment, the cell viability at the end of the production process comprises a viability of about 99.9%.

In another embodiment, transdifferentiated primary liver cells comprising human insulin producing cells are stored for use in a cell-based therapy at a later date. In another embodiment, storage comprises cryopreserving the cells.

In some embodiments, harvested 3D cell clusters are dissociated into single cells. Cells can be dissociated by using any enzyme or combination of enzymes having proteolytic activity or collagenolytic activity. In some embodiments, cells are dissociated by using trypsin. In some embodiments, cells are dissociated by using Accuttase®. In some embodiments, dissociated cells are seeded under attachment conditions.

As indicated at Step 5: Quality Analysis/Quality Control. Before any use of transdifferentiated cells in a cell-based therapy, the transdifferentiated cells must undergo a quality analysis/quality control assessment. FACS analysis and/or RT-PCR may be used to accurately determine membrane markers and gene expression. Further, analytical methodologies for insulin secretion are well known in the art including ELISA, MSD, ELISpot, HPLC, RP-HPLC. In some embodiments, insulin secretion testing is at low glucose concentrations (about 2 mM) in comparison to high glucose concentrations (about 17.5 mM).

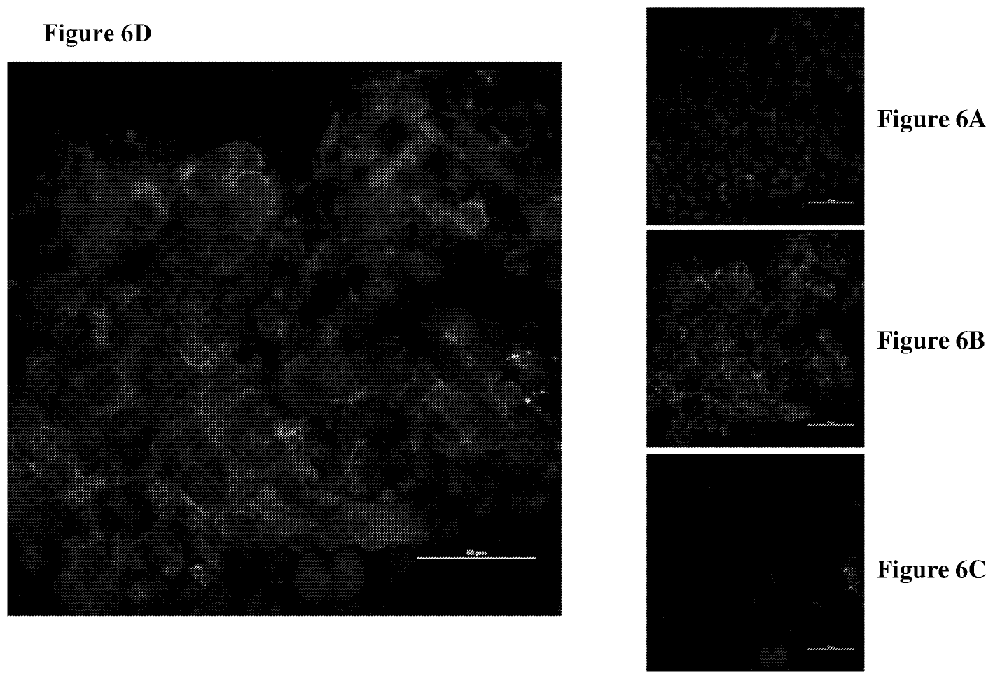

FIG. 5 shows an overview of one embodiment of a method for manufacturing an alginate scaffold loaded with bioactive peptides and cells. Embodiments for many of the steps presented in FIG. 5 are described in detail throughout this application, and will not be repeated herein, though they should be considered herein. Reference is also made to Examples 1-3, which exemplify many of these steps. In brief, the manufacturing process may be understood based on the steps presented below.

The starting materials comprise an alginate solution. In some embodiments, alginate is at a 1.2% (w/v) concentration. In some embodiments, alginate comprises sulfated alginate. In some embodiments, peptides comprising the sequence GGGGRGDY (SEQ ID NO:1) and GGGGSPPRRARVTY (SEQ ID NO:2) are bound to alginate.

As indicated in Step 1: cross-linking and freezing. In some embodiments, the alginate solution is cross-linked with a cross-linker. In some embodiments, the cross-linker is D-gluconic acid hemicalcium salt. In some embodiments, the cross-linker is added to the alginate solution in a concentration of 0.22% (w/v). In some embodiments, fifty microliters of the cross-linked alginate solution are poured into 96-well plates wells, cooled to 4° C., frozen at −20° C. for 24 h, and then lyophilized for 48 h at 0.08 bar and −57° C.

As indicated in Step 2: binding of bioactive peptides. In some embodiments, the positively charged bioactive peptides can non-covalently bind the negatively-charged sulfo or carboxyl groups on the heparin chain. In some embodiments, a bioactive peptide is bound to the scaffold by wetting dry scaffolds in a liquid medium supplemented with said bioactive peptide. In some embodiments, more than one bioactive peptide is bound the scaffold. In some embodiments, scaffolds are soaked in a liquid medium supplemented with all the bioactive peptides to be bound to the scaffold. In some embodiments, scaffolds are subsequently soaked in different liquid media, each medium supplemented with a different bioactive peptide to be bound to the scaffold.

As indicated in Step 3: attaching mammalian non-pancreatic beta cells. In some embodiments, cells are recovered from a mammalian tissue, then attached to the scaffold, and then propagated and transdifferentiated while attached to the scaffold. In some embodiments, cells are recovered from a mammalian tissue, propagated on a flask or a bioreactor, then attached to the scaffold, and then transdifferentiated while attached to the scaffold. In some embodiments, cells are recovered from a mammalian tissue, propagated and transdifferentiated on a flask or a bioreactor, and then attached to the scaffold.

In some embodiments cells are attached to the scaffold before the bioactive peptides are bound to the scaffold. In some embodiments, cells are attached to the scaffold while bioactive peptides are being bound to it. In some embodiments cells are attached to the scaffold after the bioactive peptides are bound to the scaffold. In some embodiments, cells and bioactive peptides are suspended in a medium, and the medium is applied to a scaffold. In some embodiments, drops of the suspension are dropped to the scaffolds. In some embodiments, drops of the suspension are injected into the scaffolds. In some embodiments, scaffolds are centrifuged immediately after applying the suspension. In some embodiments, scaffolds with the cells and bioactive peptides are incubated in a humidified atmosphere of 5% CO2 and 95% air, at 37° C.

Methods of Treating a Pancreatic Disorder

Disclosed herein are methods for treating a pancreatic disease or disorder in a subject, the methods comprising providing tridimensional (3D) cell clusters comprising transdifferentiated cells having a mature pancreatic beta cell phenotype, wherein at least a subset of the cells are attached to a scaffold. In some embodiments, treating a pancreatic disease or disorder comprises preventing or delaying the onset or alleviating a symptom of the disease or disorder.

In some embodiments, the 3D cell cluster is administered intradermally. In some embodiments, the 3D cell cluster is administered intraperitoneally. In some embodiments, the 3D cell cluster is administered surgically. In some embodiments, the 3D cell cluster is implanted under the left kidney capsule. In some embodiments, the 3D cell cluster is implanted in the hepatic portal vein. In some embodiments, the 3D cell cluster is implanted in the peritoneal cavity. In some embodiments, the 3D cell cluster is implanted in the omental punch. In some embodiments, the 3D cell cluster is implanted in the subcutaneous space. In some embodiments, the 3D cell cluster is administered in any combination of different routes.

A skilled artisan would appreciate that alternative sites for transplantation possess some characteristics that can make them advantageous over the hepatic portal vein, which is limited by low oxygen tension, as well as by potential inflammatory responses that can impair engraftment leading to significant losses to the implant. Table 1 describes some of the main advantages and disadvantages of the peritoneal cavity, the omental punch, and the subcutaneous space as sites for transplanting 3D cell cluster comprising transdifferentiated cells are attached to a scaffold.

TABLE 1
Comparison of different sites for transplantation

| Site for islet | | |
|---|---|---|
| transplantation | Advantages | Disadvantages |
| Peritoneal | Minimally invasive | Lack of re-innervation |
| cavity | laparoscopic procedure | of the graft |
| | Allows transplantation | Transplanted scaffolds |
| | of large islet mass | may clump |
| | | Difficult to locate all |
| | | scaffolds for harvesting |
| Omental | Exclusive portal | More complex |
| pouch | drainage | transplantation |
| | High vascular density | procedure which does not |
| | Good neoangiogenesis | allow repeated |
| | Accepting unpurified | transplantations |
| | islets | |
| | Allowing large | |
| | islet/IPC mass | |
| Subcutaneous | Easy accessibility | Poor blood supply |
| space | (minimally | |
| | invasive transplant | |
| | procedure and biopsies) | |
| | Allows transplantation | |
| | of large islet/IPC mass | |

In some embodiments, the pancreatic disorder is a degenerative pancreatic disorder. The methods disclosed herein are particularly useful for those pancreatic disorders that are caused by or result in a loss of pancreatic cells, e.g., islet beta cells, or a loss in pancreatic cell function. The subject is, in some embodiments, a mammal. The mammal can be, e.g., a human, non-human primate, mouse, rat, dog, cat, horse, or cow.

Common degenerative pancreatic disorders include, but are not limited to: diabetes (e.g., type I, type II, or gestational) and pancreatic cancer. Other pancreatic disorders or pancreas-related disorders that may be treated by using the methods disclosed herein are, for example, hyperglycemia, pancreatitis, pancreatic pseudocysts or pancreatic trauma caused by injury. Additionally, individuals whom have had a pancreatectomy are also suitable to treatment by the disclosed methods.

In some embodiments, disclosed herein is a method for treating a pancreatic disease or disorder in a subject, the method comprising administering a 3D cell cluster comprising transdifferentiated mammalian non-pancreatic beta insulin producing cells and a scaffold. In some embodiments, disclosed herein is a method for treating type I diabetes in a subject, the method comprising administering a 3D cell cluster comprising transdifferentiated mammalian non-pancreatic beta insulin producing cells and a scaffold. In some embodiments, disclosed herein is a method for treating type II diabetes in a subject, the method comprising administering a 3D cell cluster comprising transdifferentiated mammalian non-pancreatic beta insulin producing cells and a scaffold. In some embodiments, disclosed herein is a method for treating gestational diabetes in a subject, the method comprising administering a 3D cell cluster comprising transdifferentiated mammalian non-pancreatic beta insulin producing cells and a scaffold.

In some embodiments, disclosed herein is a method for treating pancreatic cancer in a subject, the method comprising administering a 3D cell cluster comprising transdifferentiated mammalian non-pancreatic beta insulin producing cells and a scaffold. In some embodiments, disclosed herein is a method for treating hyperglycemia in a subject, the method comprising administering a 3D cell cluster comprising transdifferentiated mammalian non-pancreatic beta insulin producing cells and a scaffold. In some embodiments, disclosed herein is a method for treating pancreatitis in a subject, the method comprising administering a 3D cell cluster comprising transdifferentiated mammalian non-pancreatic beta insulin producing cells and a scaffold. In some embodiments, disclosed herein is a method for treating pancreatic pseudocysts in a subject, the method comprising administering a 3D cell cluster comprising transdifferentiated mammalian non-pancreatic beta insulin producing cells and a scaffold. In some embodiments, disclosed herein is a method for treating pancreatic trauma caused by injury in a subject, the method comprising administering a 3D cell cluster comprising transdifferentiated mammalian non-pancreatic beta insulin producing cells and a scaffold. In some embodiments, disclosed herein is a method for treating a disease caused by pancreatectomy in a subject, the method comprising administering a 3D cell cluster comprising transdifferentiated mammalian non-pancreatic beta insulin producing cells and a scaffold.

Diabetes is a metabolic disorder found in three forms: type 1, type 2 and gestational. Type 1, or IDDM, is an autoimmune disease; the immune system destroys the pancreas' insulin-producing beta cells, reducing or eliminating the pancreas' ability to produce insulin. Type 1 diabetes patients must take daily insulin supplements to sustain life. Symptoms typically develop quickly and include increased thirst and urination, chronic hunger, weight loss, blurred vision and fatigue. Type 2 diabetes is the most common, found in 90 percent to 95 percent of diabetes sufferers. It is associated with older age, obesity, family history, previous gestational diabetes, physical inactivity and ethnicity. Gestational diabetes occurs only in pregnancy. Women who develop gestational diabetes have a 20 percent to 50 percent chance of developing type 2 diabetes within five to 10 years.

A subject suffering from or at risk of developing diabetes is identified by methods known in the art such as determining blood glucose levels. For example, a blood glucose value above 140 mg/dL on at least two occasions after an overnight fast means a person has diabetes. A person not suffering from or at risk of developing diabetes is characterized as having fasting sugar levels between 70-110 mg/dL.

Symptoms of diabetes include fatigue, nausea, frequent urination, excessive thirst, weight loss, blurred vision, frequent infections and slow healing of wounds or sores, blood pressure consistently at or above 140/90, HDL cholesterol less than 35 mg/dL or triglycerides greater than 250 mg/dL, hyperglycemia, hypoglycemia, insulin deficiency or resistance. Diabetic or pre-diabetic patients to which the compounds are administered are identified using diagnostic methods know in the art.

Hyperglycemia is a pancreas-related disorder in which an excessive amount of glucose circulates in the blood plasma. This is generally a glucose level higher than (200 mg/dl). A subject with hyperglycemia may or may not have diabetes.

Pancreatic cancer is the fourth most common cancer in the U S, mainly occurs in people over the age of 60, and has the lowest five-year survival rate of any cancer. Adenocarcinoma, the most common type of pancreatic cancer, occurs in the lining of the pancreatic duct; cystadenocarcinoma and acinar cell carcinoma are rarer. However, benign tumors also grow within the pancreas; these include insulinoma—a tumor that secretes insulin, gastrinoma—which secretes higher-than-normal levels of gastrin, and glucagonoma—a tumor that secretes glucagon.

Pancreatic cancer has no known causes, but several risks, including diabetes, cigarette smoking and chronic pancreatitis. Symptoms may include upper abdominal pain, poor appetite, jaundice, weight loss, indigestion, nausea or vomiting, diarrhea, fatigue, itching or enlarged abdominal organs. Diagnosis is made using ultrasound, computed tomography scan, magnetic resonance imaging, ERCP, percutaneous transhepatic cholangiography, pancreas biopsy or blood tests. Treatment may involve surgery, radiation therapy or chemotherapy, medication for pain or itching, oral enzymes preparations or insulin treatment.

Pancreatitis is the inflammation and autodigestion of the pancreas. In autodigestion, the pancreas is destroyed by its own enzymes, which cause inflammation. Acute pancreatitis typically involves only a single incidence, after which the pancreas will return to normal. Chronic pancreatitis, however, involves permanent damage to the pancreas and pancreatic function and can lead to fibrosis. Alternately, it may resolve after several attacks. Pancreatitis is most frequently caused by gallstones blocking the pancreatic duct or by alcohol abuse, which can cause the small pancreatic ductules to be blocked. Other causes include abdominal trauma or surgery, infections, kidney failure, lupus, cystic fibrosis, a tumor or a scorpion's venomous sting.

Symptoms frequently associated with pancreatitis include abdominal pain, possibly radiating to the back or chest, nausea or vomiting, rapid pulse, fever, upper abdominal swelling, ascites, lowered blood pressure or mild jaundice. Symptoms may be attributed to other maladies before being identified as associated with pancreatitis.

It should be understood that the disclosure presented herein is not limited to the particular methodologies, protocols and reagents, and examples described herein. The terminology and examples used herein is for the purpose of describing particular embodiments only, for the intent and purpose of providing guidance to the skilled artisan, and is not intended to limit the scope of the disclosure presented herein.

### EXAMPLES

### Example 1: General Methods

Human liver cells: Adult human liver tissues were obtained with the approval from the Committee of Clinical Investigations (Institutional Review Board). The isolation of human liver cells was performed as described (Sapir et al, (2005) Proc Natl Acad Sci USA 102: 7964-7969; Meivar-Levy et al, (2007) Hepatology 46: 898-905). Liver cells were cultured in Dulbecco's minimal essential medium (1 g/l of glucose) supplemented with 10% fetal calf serum, 100 units/ml penicillin; 100 ng/ml streptomycin; 250 ng/ml amphotericin B (Biological Industries, Israel) at 37° C. in a humidified atmosphere of 5% CO₂ and 95% air.

Viral infection and transdifferentiation: The adenoviruses used in this study were as follows: Ad-CMV-Pdx-1 (Sapir et al, 2005 ibid; Meivar-Levy et al, 2007 ibid), Ad-CMV-MafA (generous gift from Newgard, C.B., Duke University), and Ad-NeuroD1 (WO2016108237A1). The viral particles were generated by standard protocols (He et al, (1998) Proc Natl Acad Sci USA 95: 2509-2514). The MOIs were: Ad-CMV-Pdx-1 (1000 MOI), Ad-CMV-MafA (10 MOI) and Ad-NeuroD1 (250 MOI) unless specified otherwise. Viruses were manufactured either by OD260 Inc. (ID, USA) or by Pall Inc. (USA). Cells were infected on day 1 with a single adenoviral vector encoding PDX-1 and NeuroD1 and then seeded on standard 6 wells plates in transdifferentiation medium (TM) consisting of DMEM supplemented with 1 g/l of glucose, 10% fetal calf serum, 100 units/ml penicillin; 100 ng/ml streptomycin; 250 ng/ml amphotericin B, 10 mM nicotinamide (Sigma, Israel), 20 ng/ml EGF (Cytolab, Israel), and 5 nM Exendin 4 (Ex4). On day 4 cells were harvested, infected with Ad-CMV-MafA and seeded. Cells were grown in TM.

DNA quantification: DNA was quantified by Hoechst staining Cells were extracted from scaffolds by placing each scaffold in Eppendorf, adding 200 μl sodium citrate, shaking for 10 minutes at room temperature, and dissolving the scaffolds by pipetting. Eppendorfs were then centrifuged 600 rpm×10 min and supernatants discarded. Cells were lysed by adding 100 μl SDS 0.02% in SSC followed by 1 hour incubation at 37° C. 100 μl Hoechst solution (2 ug/ml) was added and lysates incubated for 10 min at 37° C. Fluorescence was read in 180 μl lysates at 352/461 (nm).

RNA isolation, RT and RT-PCR reactions: Cells were extracted from the scaffolds as for the DNA quantification protocol. RNA was isolated with EZ-RNA Total RNA Isolation Kit (Biological Industries, Israel), according to manufacturer's instructions. RNA concentration and purity were validated by NanoDrop (Nanodrop Technologies, USA). cDNA synthesis and real time PCR (RT-PCR) were performed according to standard protocols. GapdH and TBP were used as housekeeping genes. 2 independent duplicates were run for RT-PCR. Table 2 shows primers used for RT-PCR.

TABLE 2
RT-PCT Primers.

| Primer name | Forward | Reverse |
|---|---|---|
| TBP_MTC | TGCACAGGAG | CACATCACAG |
| | CCAAGAGTG | CTCCCCACCA |
| | AA | (SEQ ID |
| | (SEQ ID | NO: 10) |
| | NO: 9) | |
| GAPDH | ACCCACTCC | CTGTTGCTGT |
| | TCCACCTTT | AGCCAAATT |
| | GA | CGT |
| | (SEQ ID | (SEQ ID |
| | NO: 11) | NO: 12) |
| PDX-1_ORG | AAGTCTAC | GTAGGCGCC |
| | CAAAGCTC | GCCTGC |
| | ACGCG | (SEQ ID |
| | (SEQ ID | NO: 14) |
| | NO: 13) | |
| MafA_ORG | AGCAGCGG | TTGTACA |
| | CACATTCT | GGTCCCG |
| | GG | CTCTTTG |
| | (SEQ ID | (SEQ ID |
| | NO: 15) | NO: 16) |

C-peptide and insulin secretion detection: C-peptide and insulin secretion is measured by static incubations of cultured cells 6 or 7 days following the initial exposure to the viral treatment, as described (Sapir et al, (2005) ibid; Meivar-Levy et al, (2007) ibid; Aviv et al, (2009) ibid). Glucose-regulated C-peptide and insulin secretion (GSIS) is measured at 2 mM (low) and 17.5 mM (high) glucose, the latter is determined by dose-dependent analyses to maximally induce insulin secretion from transdifferentiated liver cells without having adverse effects (Sapir et al, (2005) ibid; Meivar-Levy et al, (2007) ibid; Aviv et al, (2009) ibid). C-peptide secretion is detected by radioimmunoassay using the human C-peptide radioimmunoassay kit (Linco Research, St. Charles, Mo.; <4% cross-reactivity to human proinsulin). Insulin secretion is detected by radioimmunoassay using human insulin radioimmunoassay kit (DPC, Angeles, Calif.; 32% cross-reactivity to human proinsulin). Cells grown in non-adherent conditions are transferred to adherent 6 wells plates prior to the assay.

Cell Viability and morphology: Cell viability was measured with PrestoBlue™ (ThermoFisher, USA) according to manufacturer's instructions. Scaffolds were transferred to 48-well plates were 0.3 ml of reagent diluted in culture medium was added. Plates were incubated at 37° C. in a CO2 humified incubator for 2 hours. After incubation, 250 μl from the medium were transferred to a flourimetric 96-well plate, were fluorescence and absorbance were read.

Encapsulation: Encapsulation agents encompass encapsulation agents known and available in the art, such as alginate, cellulose sulphate, collagen, chitosan, gelatin, agarose, polyethylene glycol (PEG), poly-L-lysine (PLL), polysulphone (PSU), polyvinyl alcohol (PVA), polylactic acid (PLA), acrylates, or low molecular weight dextran sulphate (LMW-DS)

### Example 2: Construction of Alginate/Alginate Sulfate Scaffolds Loaded with Insulin-Producing Cells and Growth Factors VEGF, IL-10, PDGF-ββ and TGF-β1

The aim of this study was to grow transdifferentiated insulin producing cells on alginate/alginate sulfate scaffolds.

Methods: Scaffold Preparation

Alginate scaffolds were prepared as described in U.S. Pat. No. 7,517,856 and Orr et al, (2016) Acta Biomater. 45:196-209. 0.56 ml 1.42% LVG54 alginate (NovaMatrix FMC Biopolymers, Norway) were mixed with 0.14 ml 1.42% VLVG alginate (NovaMatrix FMC Biopolymers, Norway). 0.7 ml of the alginate mix was mixed with 0.2 ml 0.81% Ca-gluconate (Ca-glu) using a homogenizer for about 3 min at 26-28 rpm until the solution was homogeneous.

A mix of proteins TGFβ, IL-10, VEGF, PDGFbb (MP) was used for these studies. 200 ng of each protein diluted in 12.5 μl DDW was mixed with 50 μl 4.4% alginate sulfate and incubated for 1.5 hours at 37° C. in a water bath. 100 μl of the alginate sulfate-protein mix were then mixed with 900 μl of the alginate Ca-glu mix at room temperature while stirring for 5-10 min 50-100 μl of the solution were poured in 96-plate wells (Corstar Cat 3596, Corning Inc., USA). The final percentage of alginate was 1%, of sulfated alginate was 0.2%, and of Ca was 0.16%. Plates were cooled at 4° C. overnight and then at −20° C. overnight.

Cell Seeding

Cells were infected on day 1 with an adenoviral vector encoding PDX-1 and NeuroD1. On day 4, cells were harvested and infected with Ad-CMV-MafA. On day 6 cells were resuspended in TM medium. 15μl of medium comprising 0.5×10⁶, 1×10⁶, or 2.5×10⁶ cells were seeded in scaffolds previously placed in 96 well-plates. The media containing the cells was pipetted into the center of the scaffolds. Plates were then centrifuged at 100 g for 2 min at 25° C., and then incubated in a humified CO2 incubator for 30 min. Afterwards, 50 μl medium was added every 20 min until reaching a 100μl final volume, and then incubated further 30 min After scaffolds were completely wet, they were transferred into 12 well plates using a spatula. 1 ml of medium was added to each well, and plates were then incubated in a humified CO2 incubator for the time required.

Results

Cell Viability. Viability of transdifferentiated cells on scaffolds was measured shortly after seeding (0 h), and following 24 h, 48 h, and 72 h by a PrestoBlue™ assay, and was compared to viability of transdifferentiated cells cultured in regular 6-well plates. 0.5×10⁶, 1×10⁶, and 2.5×10⁶ cells were seeded on scaffolds, and 0.5×10⁶ were seeded in well plates. The PrestoBlue™ assay revealed a high correlation between the number of cells seeded and the observed metabolic activity at 0 h. At 24 h the correlation was less marked, probably due limited diffusion of nutrients and oxygen caused by cell aggregation and clustering. Tables 3 and 4 show two independent cell viability experiments.

TABLE 3
Metabolic activity of transdifferentiated cells (PrestoBlue ™ assay).
Results are presented as absorbance of sample/absorbance
of blank (empty scaffold). Three independent measurements
are presented for each timepoint.

| | | Scaffold | Scaffold | Scaffold | 2D (6-well plate) |
|---|---|---|---|---|---|
| | | 2.5 × 10⁶ | 1 × 10⁶ | 0.5 × 10⁶ | 0.5 × 10⁶ |
| | Time | cells | cells | cells | cells |
| OD1 | 0 h | 10916 | 6341 | 2398 | 3523 |
| OD2 | 0 h | 10517 | 7034 | 2903 | 3445 |
| OD3 | 0 h | 10873 | 6181 | 2468 | — |
| OD1 | 24 h | 9916 | 6063 | 2374 | 5867 |
| OD2 | 24 h | 9177 | 5792 | 1811 | 5295 |
| OD3 | 24 h | 10113 | 6387 | 1833 | — |

TABLE 4
Metabolic activity of transdifferentiated cells (PrestoBlue ™ assay).
Results are presented as absorbance of sample/absorbance
of blank (empty scaffold). Three independent measurements
are presented for each timepoint.

| | | Scaffold | 2D (6-well plate) |
|---|---|---|---|
| | | 2.5 × 10⁶ | 0.5 × 10⁶ |
| | Time | cells | cells |
| OD sc. 1 | 0 h | 13106 | — |
| OD sc. 2 | 0 h | 9550 | — |
| OD sc. 3 | 0 h | 11136 | — |
| OD sc. 1 | 24 h | 10606 | 4852 |
| OD sc. 2 | 24 h | 10207 | 5047 |
| OD sc. 3 | 24 h | 11105 | — |
| OD sc. 1 | 48 h | 10914 | 7017 |
| OD sc. 2 | 48 h | 10279 | 6405 |
| OD sc. 3 | 48 h | 12139 | — |
| OD sc. 1 | 72 h | 10082 | 7465 |
| OD sc. 2 | 72 h | 8916 | 5617 |
| OD sc. 3 | 72 h | 11633 | |

DNA contents: DNA content was measured shortly after seeding (0 h), 24 h, 48 h, and 72 h after seeding by Hoechst staining following cell lysis. Hoechst staining revealed a high correlation between the number of cells seeded and the DNA content at all time points. Tables 5 and 6 show two independent experiments measuring DNA content.

TABLE 5
DNA content in transdifferentiated cells (Hoechst staining). DNA
content was measured after cell lysis. Results are presented as
absorbance of sample/absorbance of blank (empty scaffold). Three
independent measurements are presented for each timepoint.

| | | Scaffold | Scaffold | Scaffold | 2D (6-well plate) |
|---|---|---|---|---|---|
| | | 2.5 × 10⁶ | 1 × 10⁶ | 0.5 × 10⁶ | 0.5 ×10⁶ |
| 0 h | Time | cells | cells | cells | cells |
| OD1 | 0 h | 2296 | 1471 | 1031 | 1053 |
| OD2 | 0 h | 2278 | 1584 | 1082 | 1098 |
| OD3 | 0 h | 2286 | 1491 | 1015 | — |
| OD1 | 24 h | 2222 | 1446 | 1029 | 1022 |
| OD2 | 24 h | 2225 | 1471 | 1057 | 1075 |
| OD3 | 24 h | 2245 | 1480 | 990 | — |

TABLE 6
DNA content in transdifferentiated cells (Hoechst staining). DNA
content was measured after cell lysis. Results are presented as
absorbance of sample/absorbance of blank (empty scaffold). Three
independent measurements are presented for each timepoint.

| | | Scaffold | 2D (6-well plate) |
|---|---|---|---|
| | | 2.5 × 10⁶ | 0.5 × 10⁶ |
| | Time | cells | cells |
| OD sc. 1 | 0 h | 3224 | — |
| OD sc. 2 | 0 h | 3594 | — |
| OD sc. 3 | 0 h | 3158 | — |
| OD sc. 1 | 24 h | 3648 | 1096.5 |
| OD sc. 2 | 24 h | 2804 | 1095 |
| OD sc. 3 | 24 h | 2198 | — |
| OD sc. 1 | 48 h | 2580 | 769.5 |
| OD sc. 2 | 48 h | 2696 | 1057.5 |
| OD sc. 3 | 48 h | 2730 | — |
| OD sc. 1 | 72 h | 2428 | 982.5 |
| OD sc. 2 | 72 h | 1878 | 795 |
| OD sc. 3 | 72 h | 2906 | — |

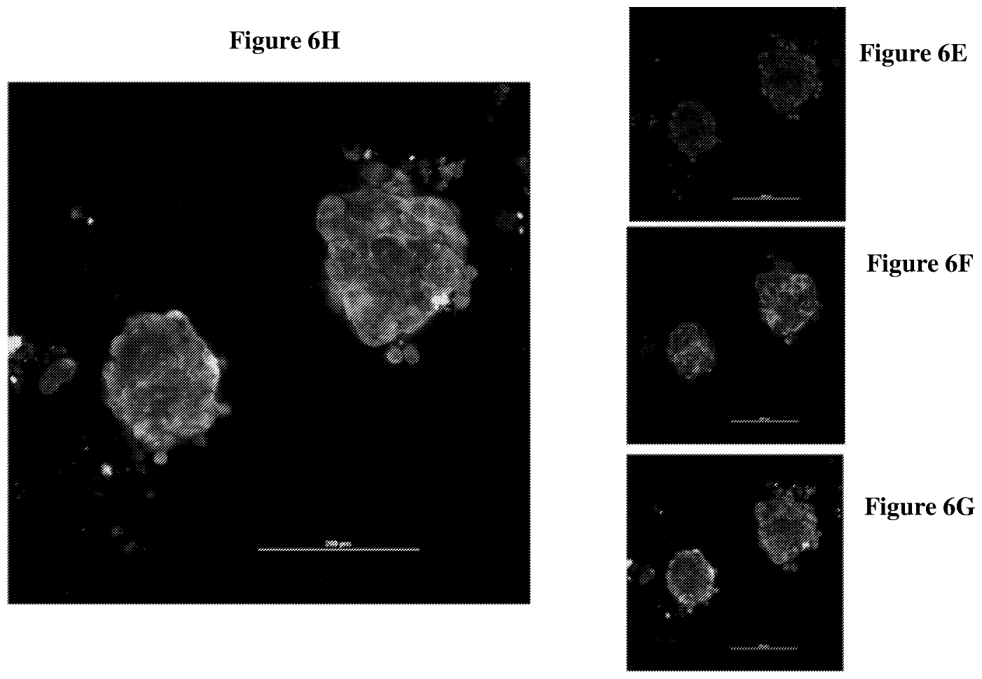

Ectopic genes expression. Cells were harvested 4 h or 24 h after seeding. RT-PCR showed expression of ectopically expressed PDX1, and MafA indicating that cells were successfully transfected. Table 7 shows PDX-1 and MafA relative quantification (RQ) to whole pancreatic cDNA. Two independent experiments were realized (Experiment #1 and Experiment #2). Confocal microscopy confirmed ectopic gene expression 4 h (FIGS. 6A-6D) and 72 h (FIGS. 6E-6H) after seeding.

TABLE 7
Expression of ectopic genes in transdifferentiated cells.

| | Repli- | Experi- | | | | | | |
|---|---|---|---|---|---|---|---|---|
| Time | cate | ment | Ct- | Ct- | Ct- | Ct- | RQ | RQ |
| (hours) | # | # | TBP | GAPDH | PDX1 | MafA | PDX1 | MafA |
| 4 | 1 | 1 | 22.7 | 15.5 | 16.1 | 16.7 | 62 | 602 |
| 4 | 2 | 1 | 23.2 | 16.2 | 16.1 | 16.1 | 95 | 1442 |
| 24 | 1 | 1 | 23.2 | 16.4 | 16.9 | 16.5 | 56 | 1204 |
| 24 | 2 | 1 | 23.0 | 15.4 | 15.9 | 15.6 | 74 | 1386 |
| 4 | 1 | 2 | 24.1 | 16.5 | 16.2 | 16.8 | 126 | 1298 |
| 4 | 2 | 2 | 26.7 | 17.9 | 17.7 | 19.1 | 186 | 1100 |
| 24 | 1 | 2 | 27.4 | 19.6 | 18.9 | 20.0 | 184 | 1325 |
| 24 | 2 | 2 | 26.7 | 17.6 | 18.7 | 18.5 | 85 | 1433 |
| | PC | — | 25.0 | 19.1 | 25.0 | 28.9 | 1 | 1 |

Ct: cycle threshold.
RQ: relative quantification to whole pancreatic cDNA, which is the positive control.
PC: positive control (pancreatic cDNA).

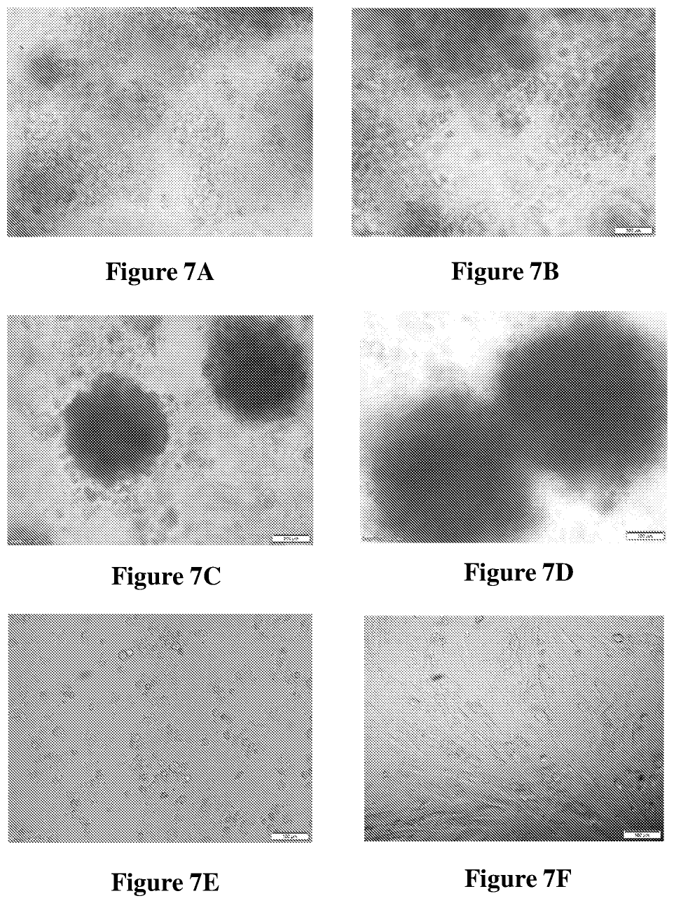

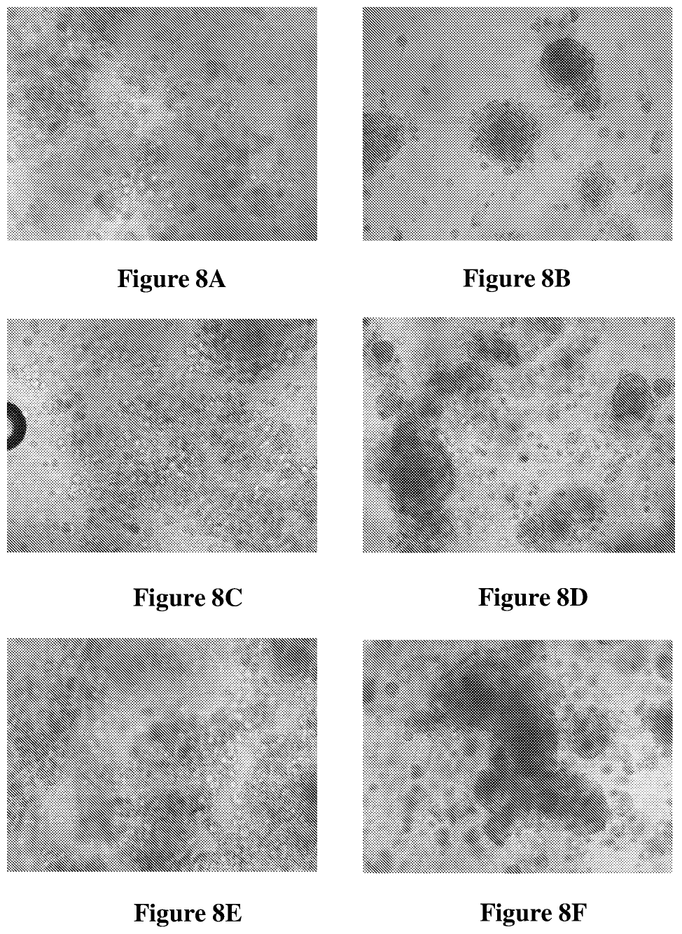

Formation of Cell Clusters. Light microscopy revealed the formation of clusters of transdifferentiated cells 4 h and 72 h after seeding on scaffolds (FIGS. 7A-7D). Transdifferentiated cells seeded on adherent 6 well plates did not form clusters (FIGS. 7E-7F). Cluster size was positively correlated with the number of cells seeded 24 h after seeding (FIGS. 8A-8F). Clusters of cells of about 200 μm diameter were observed in scaffolds 72 h after seeding 2.5×10⁶ transdifferentiated cells (FIGS. 7C and 7D). The observed tridimensional clusters improve cell functioning, as they resemble the natural environment of the cell better than two-dimensional (2D) cultures.

Conclusions. IPCs were efficiently seeded in macroporous alginate scaffolds with minimal cell loss during seeding. The DNA content in IPCs seeded on scaffold at 0 h was similar to that of IPCs seeded on plates. It was observed that scaffolds can be loaded with 2.5×10⁶ cells/scaffold. The results indicate that cell seeding density can be further increased.

When seeded on plates, IPCs adhere to the surface and spread. However, when seeded on scaffolds, IPCs form 3D cell clusters in the pores of the scaffold, as revealed by light microscopy (FIG. 7).

### Example 3: Optimization of Scaffolds Loaded with Insulin Producing Cells

Alginate scaffolds loaded with insulin producing cells will be produced by different protocols and the functioning of the loaded cells will be studied. Cell parameters to be analyzed include cell viability, cell morphology, cell attachment to the scaffold, and glucose stimulated insulin secretion. Cell attachment to the scaffold will be studied by analyzing the number of cells in the scaffold at different timepoints, for example by quantifying the DNA content of a crude cellular homogenate using DAPI staining. Cell viability, cell morphology and glucose stimulated insulin secretion assays will be executed as detailed in Examples 1 and 2. Transdifferentiated cells will be further characterized by gene expression and immnostaining for pancreatic hormones as detailed in Examples 1 and 2.

In order to optimize cell growing and functioning on scaffolds, cells will be seeded in scaffolds under different conditions and cell phenotype will be studied.

Number of cells seeded on scaffold. Experiments will be conducted in scaffolds loaded with 0.1×10⁶, 1×10⁶, and 10×10⁶ transdifferentiated cells.

Scaffold size. Experiments will be conducted in scaffolds ranging from 6 mm to 22 mm diameter in the base, which corresponds to 96-well plates and to 12-well plates, respectively. Scaffold height will vary from 1 mm to 10 mm.

Culture media. The addition of soluble factors to the medium will be studied.

Time between cell transdifferentiation and seeding. Experiments will be conducted in cells seeded immediately following differentiation and up to 1 week following transdifferentiation.

Time between cell seeding and implantation. In vivo experiments will be conducted in which animals will be implanted with scaffolds immediately after cell seeding and up to 2 weeks after cell seeding on scaffolds.

Seeding of other cell types. Experiments will be conducted in which mesenchymal stem cells (MSCs) and epithelial endothelial progenitor cells (EPCs), together with IPCs.

### Example 4: Survival and Vascularization Following Implantation of Transdifferentiated Cells in Alginate Scaffolds

The objectives of this study are 1) observing whether implants of scaffolds loaded with transdifferentiated cells are well tolerated, and 2) assessing the vascularization of said implants.

Alginate scaffolds loaded with VEGF and PDGF-ββ peptides and transdifferentiated cells are prepared as described in Example 2. Ten (10) w.o. male athymic nude rats are divided in 4 groups (n=3 in each group). Group 1 is implanted with 1×10⁶ transdifferentiated cells loaded in scaffolds for 1 week. Group 2 is implanted with 1×10⁶ transdifferentiated cells loaded in scaffolds for 4 weeks. Group 3 is implanted with 2×10⁶ transdifferentiated cells loaded in scaffolds for 1 week. Group 4 is implanted with 2×10⁶ transdifferentiated cells loaded in scaffolds loaded for 4 week.

Previous to implantation, rats are handled for 1 or 4 weeks (2 or 5 weeks including one-week acclimation). Recipient nude male rats (˜200 g) are anesthetized with a combination of ketamine (40 mg/kg) and xylazine (10 mg/kg). A midline abdominal incision will be made, and transplants (3 per animal) are placed on the omentum and secured in place by wrapping.

Animals are monitored for clinical signs twice a week until study termination. Body weight will be monitored once during acclimation, before surgical procedure and twice a week thereafter Animals are monitored daily for morbidity and mortality.

Either 1 or 4 weeks after implantation, the omentoum with the integrated scaffolds are collected and placed in 4% formaldehyde or PFA 2.5%. Implants are evaluated by histopathology, comprising H&E staining and immunohistochemistry for human cells marker (Ku80), angiogenesis/early blood vessels formation (CD31) and pancreatic transcription factor expression (PDX-1).

### Example 5: Survival, Potency and Vascularization Following Implantation of Transdifferentiated Cells in Alginate Scaffolds

The objectives of this study are 1) assessing the vascularization of alginate scaffold implants, 2) assessing the immunotolerance induced by bioactive polypeptides loaded into the scaffold, 3) assessing the in vivo effect of alginate scaffolds loaded with polypeptides to transdifferentiated cells viability and function, 4) assessing the effect of implants on blood insulin.

Alginate scaffolds loaded with VEGF, PDGF-ββ, IL-10 and TGF-β1 peptides and transdifferentiated cells are prepared as described in Example 2. Eight (8) w.o. female athymic nude female SCID mice, Beige mice, or male athymic nude rats are divided in 3 groups. Group 1 (n=12) is implanted with 3×10⁶ transdifferentiated cells loaded in an alginate scaffold loaded with VEGF, PDGF-ββ, IL-10 and TGF-β1 peptides. Group 2 (n=12) is implanted with 3×10⁶ transdifferentiated cells loaded in an alginate scaffold without VEGF, PDGF-ββ, IL-10 and TGF-β1 peptides. Group 3 (n=6) is implanted with an alginate scaffold without cells.

Previous to implantation, mice will are handled for 8 weeks (9 weeks including one-week acclimation). Anesthetized mouse are placed with both flanks exposed. Scaffolds are implanted in the subcutaneous space in two separate locations (top and bottom, one device per each flank, 6×10⁶ cells per animal), at a volume of 100 μL per area.

Animals are monitored for clinical signs, twice a week until study termination. Body weight is monitored once during acclimation, before surgical procedure and twice a week thereafter. Animals are monitored daily for morbidity and mortality.

Half of the animals are sacrificed 4 weeks after transplantation, and the other half are sacrificed 8 weeks after transplantation. Before sacrifice, food is taken out early in the morning, and 6 hours later a bolus of glucose (3 gr/kg) is administered IP. Blood is collected 30 minutes after glucose administration by terminal bleeding for serum preparation. Implants are collected and placed in 4% formaldehyde, PFA, or are snapped frozen. Implants are evaluated by histopathology, comprising H&E staining and immunohistochemistry for human cells marker (Ku80), human insulin, angiogenesis/early blood vessels formation (CD31 or SMA) and inflammatory markers.

1. A composition comprising a three-dimensional (3D) cell cluster comprising transdifferentiated insulin producing cells (IPC) attached to a polysaccharide matrix.

2. The composition of claim 1, wherein said polysaccharide matrix comprises a sulfated polysaccharide matrix, or a mix of sulfated polysaccharides and polysaccharides.

3. The composition of claim 1, wherein said polysaccharide matrix comprises an alginate polysaccharide, alginate sulfate, hyaluronan sulfate, or a combination thereof.

4-22. (canceled)

23. A method of generating a three-dimensional (3D) cell cluster comprising transdifferentiated insulin producing cells (IPC) attached to a polysaccharide matrix, the method comprising:
(a) providing the polysaccharide matrix;
(b) providing a human tissue;
(c) processing said tissue to recover primary human cells;
(d) propagating and expanding the cells of step (c) to a predetermined number of cells;
(e) transdifferentiating the cells of step (d); and
(f) attaching at least a subset of said cells to said polysaccharide matrix; thereby generating a 3D cell cluster comprising transdifferentiated insulin producing cells attached to a polysaccharide matrix.

24. The method of claim 23, wherein at step (e) said transdifferentiating comprises infecting said cells with:
(a) a first adenoviral vector comprising a nucleic acid encoding a human PDX-1 polypeptide;
(b) a second adenoviral vector comprising a nucleic acid encoding a second human pancreatic transcription factor polypeptide; and
(c) a third adenoviral vector comprising a nucleic acid encoding a human MafA polypeptide.

25. The method of claim 24, wherein said second pancreatic transcription factor is selected from NeuroD1 and Pax4.

26. The method of claim 24, wherein the infections with said first adenoviral vector and said second adenoviral vector are concurrent.

27. The method of claim 23, wherein step (d), step (e), or a combination thereof are executed under non-adherent cell culture conditions.

28. The method of claim 23, wherein said polysaccharide matrix comprises a sulfated polysaccharide matrix, or a mix of sulfated polysaccharides and polysaccharides.

29. The method of claim 23, wherein said polysaccharide matrix comprises an alginate polysaccharide, alginate sulfate, hyaluronan sulfate, or a combination thereof.

30-31. (canceled)

32. The method of claim 28, wherein said sulfated polysaccharide matrix comprises a at least one bioactive polypeptide associated with a sulfate group of said sulfated polysaccharide matrix.

33. (canceled)

34. The method of claim 32, wherein said bioactive polypeptide comprises a positively-charged polypeptide, a heparin-binding polypeptide, or a combination of both.

35. The method of claim 32, wherein said bioactive polypeptide is selected from a group comprising antithrombin III (ATIII), thrombopoietin (TPO), serine protease inhibitor (SLP1), CI esterase inhibitor (C1-INH), Vaccinia virus complement control protein (VCP), a fibroblast growth factor (FGF), a FGF receptor, vascular endothelial growth factor (VEGF), hepatocyte growth factor (HGF), insulin-like growth factor (IGF), a platelet-derived growth factor (PDGF), PDGF-ββ, bone morphogenetic protein (BMP), epidermal growth factor (EGF), CXC chemokine ligand 4 (CXCL4), stromal cell-derived factor-1 (SDF-1), interleukin-6 (IL-6), interleukin-8 (IL-8), interleukin-10 (IL-10), Regulated on Activation, Normal T Expressed and Secreted (RANTES), monocyte chemoattractant protein-1 (MCP-1), macrophage inflammatory peptide-1 (MIP-1), lymphotactin, fractalkine, an annexin, apolipoprotein E (ApoE), immunodeficiency virus type-1 (HIV-1) coat protein gp120, cyclophilin A (CypA), Tat protein, viral coat glycoprotein gC, gB or gD of herpes simplex virus (HSV), an envelope protein of Dengue virus, circumsporozoite (CS) protein of Plasmodium falciparum, bacterial surface adhesion protein OpaA, 1-selectin, P-selectin, heparin-binding growth-associated molecule (HB-GAM), thrombospondin type I repeat (TSR), peptide myelin oligodendrocyte glycoprotein (MOG), amyloid P (AP), transforming growth factor (TGF)-β1, or any combination thereof.

36-37. (canceled)

38. The method of claim 32, wherein said association comprises a non-covalent bond.

39. The method of claim 24, wherein said 3D cell cluster is encapsulated by an encapsulation agent comprising a material selected from a group comprising: alginate, cellulose sulphate, collagen, chitosan, gelatin, agarose, polyethylene glycol (PEG), poly-L-lysine (PLL), polysulphone (PSU), polyvinyl alcohol (PVA), polylactic acid (PLA), acrylates, and low molecular weight dextran sulphate (LMW-DS), any derivatives thereof, and any combination thereof.

40-41. (canceled)

42. The method of claim 23, wherein said transdifferentiated cells comprise improved glucose regulated C-peptide secretion, improved glucose regulated insulin secretion, increased insulin content, or increased expression of GCG and NKX6.1, or any combination thereof, compared to transdifferentiated non-pancreatic beta insulin producing cells cultured as a 3D cell cluster without a polysaccharide matrix.

43. The method of claim 23, wherein the viability of said transdifferentiated mammalian non-pancreatic beta insulin producing cells is similar to that of transdifferentiated non-pancreatic beta insulin producing cells cultured as a monolayer cell culture.

44. The method of claim 23, wherein said transdifferentiated mammalian non-pancreatic beta insulin producing cells are adult cells selected from a group comprising: epithelial cells, endothelial cells, keratinocytes, fibroblasts, muscle cells, hepatocytes, liver cells, blood cells, stem or progenitor cells, embryonic heart muscle cells, liver stem cells, neural stem cells, mesenchymal stem cells, hematopoietic stem and progenitor cells, pancreatic cells other than pancreatic beta cells, acinar cells, alpha-cells, or any combination thereof.

45-47. (canceled)

48. A method for treating a pancreatic disease or disorder in a subject, the method comprising administering said 3D cell cluster comprising transdifferentiated insulin producing cells of claim 23.

49. (canceled)

50. The method of claim 48, wherein said pancreatic disease or disease comprises type I diabetes, type II diabetes, gestational diabetes, pancreatic cancer, hyperglycemia, pancreatitis, pancreatic pseudocysts, pancreatic trauma caused by injury, or a disease caused by pancreatectomy, or any combination thereof.
